(19) United States
(12) Patent Application Publication
(10) Pub. No.: US 2025/0069036 A1
(43) Pub. Date: Feb. 27, 2025

(54) PHARMACEUTICAL TRIALS SYSTEMS AND METHODS

(71) Applicant: R.P. Scherer Technologies, LLC, Carson City, NV (US)

(72) Inventors: Carsten STAHLHOFEN, Eppstein, Hessen (DE); Samantha Jane JONES, Mold, Flintshire (GB); Martin Bernd PUBANZ, Waiblingen (DE); Stephen FLAHERTY, Tyldesley, Greater Manchester (GB); Matthew BLUME, Overland Park, KS (US); Philip ZEMLER, Olathe, KS (US); Huzaifa MERCHANT, Bedford, MA (US); Kate Marie CAREY, Lansdale, PA (US)

(73) Assignee: R.P. Scherer Technologies, LLC, Carson City, NJ (US)

(21) Appl. No.: 18/724,812

(22) PCT Filed: Dec. 28, 2022
(86) PCT No.: PCT/US2022/082479

§ 371 Date: Jun. 27, 2024

Related U.S. Application Data
(60) Provisional application No. 63/295,050, filed on Dec. 30, 2021.

(51) Int. Cl. G06Q 10/0875; G16H 10/20

(52) U.S. Cl. CPC G06Q 10/0875; G16H 10/20

(57) ABSTRACT

Embodiments of the present disclosure include systems and methods for generating an executable batch record for a clinical trial study. The method includes displaying a user interface for having a production user input batch record information comprising via a production user interface (UI), and receiving packaging instructions, a bill of materials, and a lot assignment from the production user. The method further includes producing a preliminary batch record based on the information input by the production user. The method further displaying a customer UI that includes the preliminary batch record and an interface for approving the preliminary batch record, and receiving an approval of the preliminary batch record from a customer user. The method further includes producing an executable batch record for a producer to execute upon customer approval.

### CROSS-REFERENCE TO RELATED APPLICATIONS

This application is a U.S. national stage application under 35 U.S.C. § 371 of International Application No. PCT/US2022/082479, filed Dec. 28, 2022, which claims the benefit of U.S. Provisional Application No. 63/295,050 filed Dec. 30, 2021, the entire contents of which are incorporated herein by reference.

### FIELD OF DISCLOSURE

The present disclosure relates generally to systems and methods for generating an executable batch record for packaging output materials for clinical trial studies, and more specifically, generating a standardized electronic executable batch record for packaging output materials for clinical trial studies that can be used across multiple packaging sites.

### BACKGROUND

Companies engaging in clinical trial studies, particularly, pharmaceutical clinical trials are required to adhere to government regulated clinical packaging and labeling standards. For example, various national bodies like the Food and Drug Administration (FDA), Medicines and Healthcare products Regulatory Agency (MHRA) and European Medicines Agency (EMA) provide a regulatory framework for clinical packaging. As such, each clinical packaging job whether primary (e.g., putting bulk drug into bottle, blister, vial or similar) or secondary packaging job (e.g., clinically labeling a primary bottle and putting it into a box and labeling the box to create a patient kit) must be controlled and repeatable in action and follow appropriate regulatory guidelines.

Traditionally, clinical packaging is managed via a document known initially as a study batch record or study packaging record. The study batch record, which subsequently becomes an executed batch record or executed packaging record. Existing study batch records and executed batch records are generally captured in the form of a paper batch record. The paper batch record can be pre-approved by one or more individuals overseeing the clinical trial study. The paper batch record may then be disseminated to one or more packaging sites, and filled in manually by operators during primary or secondary packaging runs. This paper-based process, however, relies heavily on manual input, which can lead to human error, and does not produce a standardized batch record across sites. Furthermore, if a company running a clinical trial determines that additional batches should be produced, the batch record will have to be re-generated and go through the approval process again.

Accordingly, there exists a need to provide an electronic based record that can reduce the chances of human error and provide a harmonized record across various packaging sites that can efficiently scale without going through the tedious approval process.

### BRIEF SUMMARY

Embodiments of the present disclosure include systems and methods for a computer based system for generating a standardized executable batch record. Such a system is designed to allow the executable batch record to be used across multiple packaging sites (including domestic and international packaging sites), generate an executable batch record that requires minimal manual updates at a packaging site, and reduce duplicative approvals by the company in charge of the clinical trial studies. Additionally, because the study batch record is maintained and approved on a centralized system, if the company conducting the study would like to produce additional batches of output materials (e.g., produce a second run of output materials for further clinical trial studies), then the system can generate a new executable batch record based on the existing study batch record and study information (e.g., without having to regenerate a new study batch record). Furthermore, the system can maintain an audit history that tracks the actions of individuals as they interact with the system.

Embodiments of the present disclosure include systems and methods for generating an executable batch record for a clinical trial study. For example, in one or more embodiments, the method can include, at a first electronic device associated with a production facilitator, displaying a user interface (UI) for inputting batch record information, the UI including at least packaging instructions, a bill of materials, and a lot assignment. In one or more examples, the batch record information can include a selection of inventory associated with a production site and a selection of inventory associated with a pharmaceutical customer. In one or more embodiments, the method can further include, at the first electronic device, receiving the packaging instructions, the bill of materials, and the lot assignment from a user associated with the production facilitator. In one or more embodiments, the method can further include, producing a preliminary batch record based on the information input by the user associated with the production facilitator. In one or more embodiments, the method can further include, at a second electronic device associated with a pharmaceutical batch customer, displaying a customer user interface that comprises the preliminary batch record and an interface for approving the preliminary batch record. In one or more embodiments, the method can further include, at the second electronic device, receiving an approval of the preliminary batch record from a user associated with the pharmaceutical batch customer. In one or more embodiments, the method can further include, in accordance with receiving the approval of the preliminary batch record producing an executable batch record for a producer to execute.

Embodiments of the present disclosure can further include an electronic system for generating an executable batch record for a clinical trial study. The electronic system can include one or more processors, a memory, and one or more programs, the one or more programs stored in the memory and configured to be executed by the one or more processors. According to embodiments of the present disclosure, the one or more programs can include instructions for at a first electronic device associated with a production facilitator, displaying a user interface (UI) for inputting batch record information, the UI including at least packaging instructions, a bill of materials, and a lot assignment. In such embodiments, the batch record information can include a selection of inventory associated with a production site and a selection of inventory associated with a pharmaceutical customer. In such embodiments, the one or more programs can further include instructions for, at the first electronic device, receiving the packaging instructions, the bill of materials, and the lot assignment from a user associated with the production facilitator. In such embodiments, the one or more programs can further include instructions for producing a preliminary batch record based on the information input by the user associated with the production facilitator. In one or more embodiments, the one or more programs can further include instructions for, at a second electronic device associated with a pharmaceutical batch customer, displaying a customer user interface that comprises the preliminary batch record and an interface for approving the preliminary batch record. In one or more embodiments, the one or more programs can further include instructions for, at the second electronic device, receiving an approval of the preliminary batch record from a user associated with the pharmaceutical batch customer. In one or more embodiments, the one or more programs can further include instructions for, in accordance with receiving the approval of the preliminary batch record, producing an executable batch record for a producer to execute.

Embodiments of the present disclosure can further include non-transitory computer-readable storage medium storing one or more programs, the one or more programs comprising instructions, which when executed by one or more processors of one or more electronic devices having a display, cause the one or more electronic devices to perform a method. In one or more examples, the method can include at a first electronic device associated with a production facilitator, displaying a user interface (UI) for inputting batch record information, the UI including at least packaging instructions, a bill of materials, and a lot assignment. In such examples, the batch record information can include a selection of inventory associated with a production site and a selection of inventory associated with a pharmaceutical customer. In such examples, the method can further include, at the first electronic device, receiving the packaging instructions, the bill of materials, and the lot assignment from a user associated with the production facilitator. In one or more examples, the method can further include, producing a preliminary batch record based on the information input by the user associated with the production facilitator. In one or more examples, the method can further include, at a second electronic device associated with a pharmaceutical batch customer, displaying a customer user interface that comprises the preliminary batch record and an interface for approving the preliminary batch record. In one or more examples, the method can further include, at the second electronic device, receiving an approval of the preliminary batch record from a user associated with the pharmaceutical batch customer. In one or more examples, the method can further include, in accordance with receiving the approval of the preliminary batch record producing an executable batch record for a producer to execute.

### DESCRIPTION OF THE FIGURES

FIGS. 1A and 1B illustrate exemplary systems for generating an executable batch record for packaging materials for a clinical trial study, in accordance with some embodiments of this disclosure.

FIGS. 1C and 1D illustrate exemplary processes for generating an executable batch record for packaging materials for a clinical trial study, in accordance with some embodiments of this disclosure.

FIGS. 2A-2I illustrate user interfaces, in accordance with some embodiments of this disclosure.

FIG. 3A illustrates an exemplary user interface, in accordance with some embodiments of this disclosure.

FIG. 3B illustrates an exemplary process for generating a study information, in accordance with some embodiments of this disclosure.

FIGS. 3C-3L illustrate user interfaces, in accordance with some embodiments of this disclosure.

FIG. 4A illustrates an exemplary process for generating a study part, in accordance with some embodiments of this disclosure.

FIGS. 4B-4Q illustrate exemplary user interfaces, in accordance with some embodiments of this disclosure.

FIGS. 5A-1 and 5A-2 illustrate an exemplary user interface, in accordance with some embodiments of this disclosure.

FIG. 5B illustrates an exemplary process for generating a study batch record, in accordance with some embodiments of this disclosure.

FIGS. 5C-5Y illustrate exemplary user interfaces, in accordance with some embodiments of this disclosure.

FIG. 6A illustrates an exemplary process for creating packaging instructions, in accordance with some embodiments of this disclosure.

FIGS. 6B-60 illustrate exemplary user interfaces, in accordance with some embodiments of this disclosure.

FIG. 7A illustrates an exemplary process for generating a lot assignment, in accordance with some embodiments of this disclosure.

FIGS. 7B-7H illustrate exemplary user interfaces, in accordance with some embodiments of this disclosure.

FIG. 7I illustrates an exemplary process for generating a lot assignment, in accordance with some embodiments of this disclosure.

FIGS. 7J-7V illustrate exemplary user interfaces, in accordance with some embodiments of this disclosure.

FIG. 8A illustrates an exemplary process for generating an executable batch record, in accordance with some embodiments of this disclosure.

FIGS. 8B-1, 8B-2, 8B-3, 8C-1, 8C-2, and 8D-8N illustrate exemplary user interfaces, in accordance with some embodiments of this disclosure.

FIGS. 9A-9C illustrate exemplary user interfaces, in accordance with some embodiments of this disclosure.

FIGS. 10A-10C illustrate exemplary user interfaces, in accordance with some embodiments of this disclosure.

FIGS. 11A-11E illustrate exemplary user interfaces, in accordance with some embodiments of this disclosure.

FIG. 12 illustrates an exemplary electronic device, in accordance with embodiments of this disclosure.

### DETAILED DESCRIPTION

Embodiments of the present disclosure include techniques for generating an executable batch record for packaging materials for a clinical trial study. Systems, including user interfaces, according to embodiments of this disclosure are designed to allow the executable batch record to be used across multiple entities (e.g., a production facilitator and a customer) and accessed at multiple packaging sites (including domestic and international packaging sites). Further, systems according to embodiments of this disclosure are designed to generate an executable batch record that requires minimal manual updates, and reduce duplicative approvals by the customer (e.g., company in charge of the clinical trial studies). Additionally, because the study batch record is maintained and approved on a centralized, if the customer desires to produce additional batches of output materials (e.g., produce a second run of output materials for further clinical trial studies), the system can generate a new executable batch record based on the existing study batch record and study information (e.g., without having to regenerate a new study batch record).

The system can be used and accessed by customers (e.g., companies managing the clinical trial studies) and/or production facilitators (e.g., companies in charge of packaging the batches of output materials used in the clinical trial studies). For example, production facilitator can input information to generate the executable batch record into the system, while the customer can approve the information in order to generate the executable batch record.

The following description is presented to enable a person of ordinary skill in the art to make and use the various embodiments. Descriptions of specific devices, techniques, and applications are provided only as examples. Various modifications to the examples described herein will be readily apparent to those of ordinary skill in the art, and the general principles defined herein may be applied to other examples and applications without departing from the spirit and scope of the various embodiments. Thus, the various embodiments are not intended to be limited to the examples described herein and shown, but are to be accorded the scope consistent with the claims.

Although the following description uses terms “first,” “second,” etc. to describe various elements, these elements should not be limited by the terms. These terms are only used to distinguish one element from another. For example, a first graphical representation could be termed a second graphical representation, and, similarly, a second graphical representation could be termed a first graphical representation, without departing from the scope of the various described embodiments. The first graphical representation and the second graphical representation are both graphical representations, but they are not the same graphical representation.

The terminology used in the description of the various described embodiments herein is for the purpose of describing particular embodiments only and is not intended to be limiting. As used in the description of the various described embodiments and the appended claims, the singular forms “a,” “an,” and “the” are intended to include the plural forms as well, unless the context clearly indicates otherwise. It will also be understood that the term “and/or” as used herein refers to and encompasses any and all possible combinations of one or more of the associated listed items. It will be further understood that the terms “includes,” “including,” “comprises,” and/or “comprising,” when used in this specification, specify the presence of stated features, integers, steps, operations, elements, and/or components, but do not preclude the presence or addition of one or more other features, integers, steps, operations, elements, components, and/or groups thereof.

The term “if” is, optionally, construed to mean “when” or “upon” or “in response to determining” or “in response to detecting,” depending on the context. Similarly, the phrase “if it is determined” or “if [a stated condition or event] is detected” is, optionally, construed to mean “upon determining” or “in response to determining” or “upon detecting [the stated condition or event]” or “in response to detecting [the stated condition or event],” depending on the context.

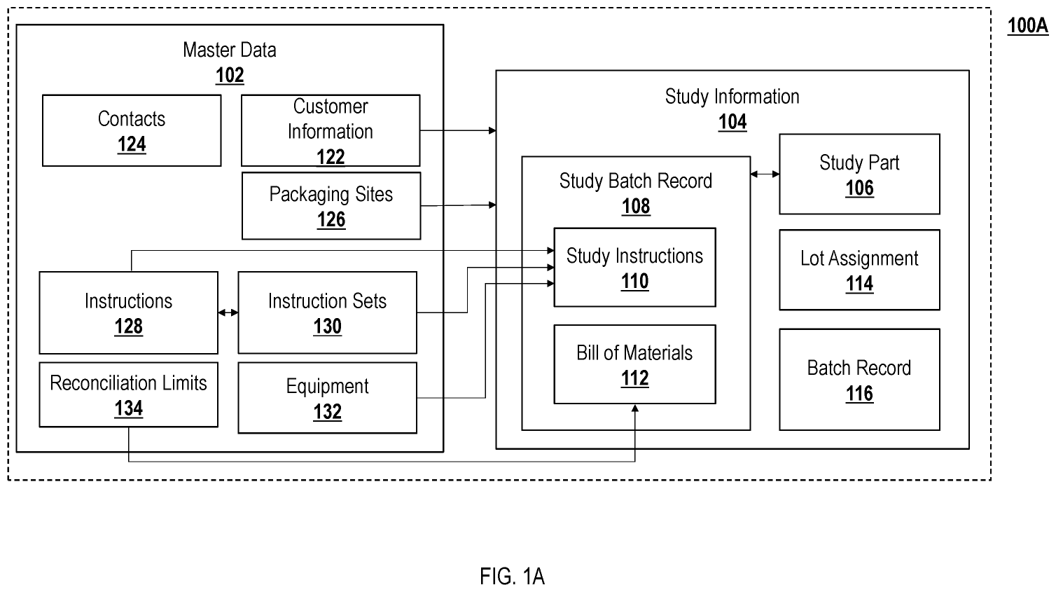

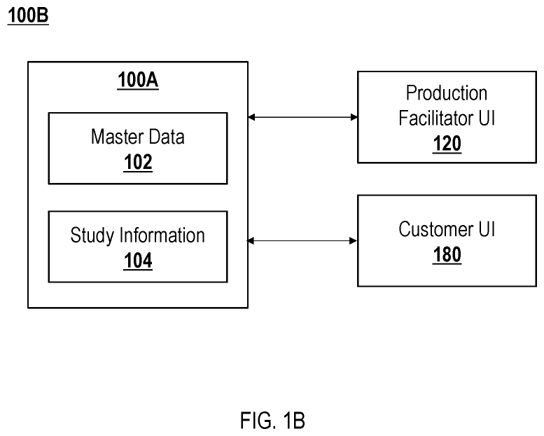

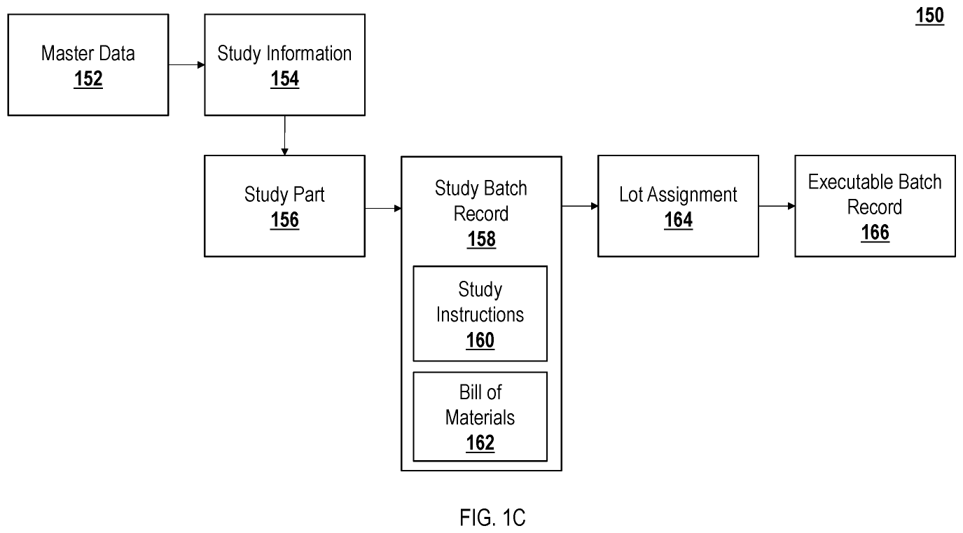

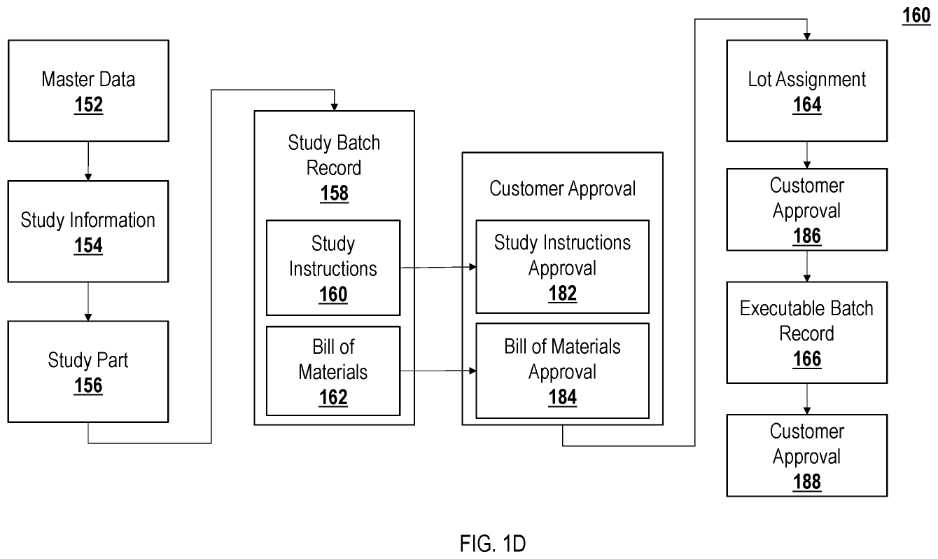

FIG. 1A illustrates an exemplary system 100A for generating an executable batch record for packaging materials for a clinical trial study, in accordance with embodiments of this disclosure. The system can include master data 102 and study information 104. The master data 102 can correspond to information related to a customer, for example, a company that runs clinical trial studies including pharmaceutical clinical trial studies. As shown in the figure, the master data 102 can include customer information 122, contacts 124, packaging sites 126, instructions 128, instruction sets 130, equipment 132, reconciliation limits 134. Additional details relating to the master data 102 will be discussed in greater detail below. In some examples, the system can include master data 102 for a plurality of different customers, where each master data corresponds to a different customer. In one or more examples, a single customer (e.g., company) can be associated with multiple instances of customer information 122, (e.g., where a company may include multiple departments that are overseeing different clinical trial studies).

The study information 104 can include one or more study parts 106, one or more study batch records 108, one or more lot assignments 114, and one or more batch records 116. To the extent that FIG. 1A illustrates a single study part 106, a single study batch record 108, a single lot assignment 114, and a single batch record 116, a skilled artisan will understand that multiple instances of these study components can be included in the study information 104. For example, a study information can include more than one study part 106. As shown in the figure, each study batch record 108 can include study instructions 110 and a bill of materials 112. In one or more embodiments, each study batch record 108 may correspond to a study part 106.

In one or more examples, the study information 104 can access data from the master data 102, e.g., customer information 122 and packaging sites 126. In one or more examples, the study instructions 110 can access data from the master data 102, e.g., packaging instructions 128, instruction sets 130, and equipment 132. In some embodiments, the bill of materials 112 can be access data from the master data 102, e.g., reconciliation limits 134.

FIG. 1B illustrates an exemplary system 100B for generating an executable batch record for packaging materials for a clinical trial study, in accordance with embodiments of this disclosure. The exemplary system 100B can include system 100A, including master data 102 and study information 104 as described above, as well as a Production Facilitator User Interface (UI) 120 and a Customer UI 180. The Production Facilitator UI 120 can correspond to a UI used by a production facilitator (e.g., a company associated with packaging the clinical trial study materials). The customer UI 180 can correspond to a UI used by a customer (e.g., a company associated with running the clinical trial study). As shown in the figure, information from system 100A can be accessed by and used to create the Production Facilitator UI 120 and the Customer UI 180. Likewise, information from the Production Facilitator UI 120 and the Customer UI 180 can be used to update information included in system 100A.

FIG. 1C illustrates an exemplary process 150 for generating an executable batch record for packaging materials for a clinical trial study, in accordance with some embodiments. Process 150 is performed, for example, using one or more electronic devices implementing a software platform. In some examples, process 150 is performed using a client-server system, and the blocks of process 150 are divided up in any manner between the server and a client device. In other examples, the blocks of process 150 are divided up between the server and multiple client devices. In other examples, process 150 is performed using only a client device or only multiple client devices. In process 150, some blocks are, optionally, combined, the order of some blocks is, optionally, changed, and some blocks are, optionally, omitted. In some examples, additional steps may be performed in combination with the process 150. Accordingly, the operations as illustrated (and described in greater detail below) are exemplary by nature and, as such, should not be viewed as limiting. In one or more examples, the process 150 can correspond to a process associated with the Production Facilitator UI.

At Block 152, the system can access master data, e.g., master data 102, associated with a customer planning to conduct a clinical trial study. As used herein, the system can refer to either system 100A or 100B. For example, the system can access the customer data 122 associated with the master data 102 of the customer. In some examples, the system can also access the packaging site data 126 associated with the master data 102 of the customer. In one or more examples, the master data for the customer can be input into the system by an individual associated with the production facilitator. In this manner, the information associated with the master data can be accessible to the system upon receiving an indication that a customer is planning to conduct a clinical trial.

At Block 154, the system can create study information, e.g., study information 104. For example, the system can obtain study information corresponding to a clinical trial study to be run by the customer. For example, one or more individuals associated with the production facilitator can input the study information associated with the clinical trial study via the Production Facilitator UI 120. The study information 104 can include, but is not limited to, information associated with packaging sites, destination countries, study languages, output materials, dosage codes, business units, and study approvers. For example, the Production Facilitator UI can obtain inputs corresponding to facilitator can input one or more packaging sites, destination countries, study languages, output materials, dosage codes, business units, and approvers from one or more individuals associated with the production facilitator.

The approvers designated in Block 154 can correspond to customer approvers, e.g., individuals associated with the customer that are authorized to approve components associated with the study information. As used herein, individuals associated with the production facilitator may be referred to as production users or production facilitator users, while individuals associated with the customer may be referred to as customer users.

In one or more examples, the packaging sites can correspond to one or more sites that package output materials for the customer's clinical trial studies. The destination countries can correspond to the countries associated with the packaging sites. The study languages can correspond to languages associated with the destination countries. For example, the system can receive an indication from a production user a study will be utilizing packaging sites in Philadelphia and Berlin, the destination countries may correspond to the US and Germany, while the study languages may correspond to English and German.

In one or more examples, the output materials may correspond to the materials, e.g., pharmaceutical drugs, provided to the participants of the clinical trial study. The dosage codes may correspond to specific dosages that participants of the clinical trial study should adhere to during the study. For example, the output materials may correspond to a bottle containing 60 tablets of a particular drug M. The dosage code may correspond to dosage instructions for the participant, e.g., take two tablets daily, one in the morning and one in the evening.

The business units may correspond to departments associated with the customer that are associated with the study. The approvers may correspond to specific customer users who are overseeing the clinical trial study and who have the authorization to approve one or more stages of the process 150. The study information 104 may also include which portions of the process 150 should receive approval from the approvers. In one or more embodiments, a customer approver can be designated to approve one or more of the study instructions 110, bill of materials 112, lot assignment 114, and batch record 116.

In one or more examples, the study information 104 can be retrieve the customer information 122 and the packaging sites 126 accessed at Block 152. For example, the study information 104 can be associated with customer information 122 and packaging sites 126 associated with the customer. In this manner, the system can automatically import data from the master data 102 (e.g., customer information 122 and packaging sites 126) associated with the customer to populate data for related to the study information 104. For example, if the customer is associated with packaging sites in Philadelphia, Berlin, and Seoul, information regarding these packaging sites can be automatically imported into the study information 104. In such examples, the Production Facilitator UI 120 can present the imported packaging sites to the production user. This enables the production user to select the packaging site associated with the customer without having to separately input or search for the information.

In one or more examples, the system may obtain an approval of the study information at Block 154 before progressing to the next block in process 150. In one or more examples, the approval may be provided by one or more designated production users. For example, the system may cause a display to present the completed study information, via Production Facilitator UI 120, for review by the designated production user. The system can then receive an approval by receiving the credentials (e.g., username and password) of the designated production user. In one or more examples, the system can cause a notification to be sent to the designated production user that the study information is ready for review and approval.

At Block 156, the system can obtain study part data, e.g., study part 106. A study part or study part data can correspond to one or more output materials of the study that can share the same packaging instructions. In one or more examples, output materials can share the same packaging instructions when the output materials can be packaged under the same conditions. For example, a study part can correspond to a 60-count bottle that is filled with 60 tablets that can be packaged at a controlled room temperature (CRT). In one or more examples, the study part data can include, but is not limited to, output materials, input materials, common materials, drug categorizations, alternative materials, storage temperature, time out environment details, humidity control details, light control details, label details, and blinding information. In one or more examples, the system can obtain study part data from one or more production users via the Production Facilitator UI 120.

As discussed above the output materials can correspond to the product provided to the participant of the clinical trial study. The input materials can correspond to the items used to create the output material. For example, an output material of a 60-count bottle filled with 60 tablets may correspond to input materials including, a 60-count bottle, the 60 tablets, a bottle closure, a desiccant, and a label for the bottle. The common materials may include input materials and/or labels that are common to one or more output materials. For example, if multiple output materials include a 60-count bottle, the 60-count bottle may be included as a common material.

The drug categorizations can be used to indicate whether a drug is a controlled substance or otherwise potentially hazardous. The alternative materials can correspond to materials that can be used to identify output or input materials that could be used as a replacement for a corresponding output or input material, e.g., in the case that there is insufficient inventory for the original output or input material. For example, a 70-count bottle may be used as an alternative material to the 60-count bottle. The storage temperature corresponds to the environmental temperature that should be maintained to store the input and/or output materials. The time out environment details corresponds to the amount of time that the input and/or output materials may be exposed to temperatures outside the storage temperature without affecting the efficacy of the drugs in the input and/or output materials. The humidity control details corresponds to the level of humidity that should be maintained for the input and/or output materials. The light control details corresponds to the amount of light exposure that the input and/or output materials can tolerate. The label details can correspond to a general description of the label associated with the output material, including whether or not the label includes a bar code.

In one or more examples, the system may obtain an approval of the study part at Block 156 before progressing to the next block in process 150. For example, the system can cause a display to present the completed study part 106 for review by one or more designated production users via the Production Facilitator UI 120. The system can then receive an approval by receiving the credentials (e.g., username and password) of the designated production user. In one or more examples, the system can cause a notification to be sent to the designated production user that the study part is ready for review and approval.

At Block 158, the system can create a study batch record, e.g., study batch record 108. In one or more examples, the system can generate a study batch record 108 based on a completed study part 106. In one or more examples, the system can automatically generate the study batch record 108 upon receiving an approval of the completed study part 106 from a production user. In one or more examples, the study batch record 108 can act as a container for the study instructions 110 and the bill of materials 112. In one or more examples, the study batch record can be configured to link and/or display the most recent version of the study instructions 110 with the bill of materials 112.

At Block 160, the system can obtain study instructions associated with the study batch record, e.g., study instructions 110 associated with study batch record 108. For example, the Production Facilitator UI 120 can provide a user interface where a production user can enter the packaging instructions and other data associated with the packaging instructions. In one or more examples, the system can create a translation of the packaging instructions based on the destination countries and/or study languages associated with the study information 104 designated at Block 154. In one or more examples, if the system receives an update of the packaging instructions, then the system can automatically update the translations of the packaging instructions, accordingly. In this manner, the packaging instructions can be harmonized across different packaging sites regardless of the location of the packaging site.

For example, packaging instructions for typical batch records are not harmonized across different packaging sites. For example, if a product is being packaged in Philadelphia, Kansas City, and Berlin, each of these locations typically receives a separate batch record that may include different instructions. Embodiments according to this example can provide a centralized repository for the study batch record, including the packaging instructions, that can be accessed from any of the packaging sites and provide harmonized packing instructions.

In one or more examples, the system may obtain an approval of the study instructions 110. In one or more examples, the approval may be provided by one or more designated production facilitator users. For example, the system may cause a display to present the completed study information, via Production Facilitator UI 120, for review by the designated production facilitator user. In one or more embodiments, each translation of the instructions can be approved separately. The system can then receive an approval by receiving the credentials (e.g., username and password) of a production approver. In one or more examples, the system can cause a notification to be sent to a production approver that the study instructions 110 are ready for review and approval.

At Block 162, the system can obtain the bill of materials associated with the study batch record, e.g., bill of materials 112 associated with the study batch record 108. For example, the Production Facilitator UI 120 can provide a user interface where a production user can enter the update the input and output materials with inventory numbers to create the bill of materials. In one or more examples, the system can import data from study part 106, e.g., input materials, output materials, label details, etc., to generate the bill of materials 110. In one or more examples, the system can access inventory information associated with the customer and assign inventory numbers to the output materials and/or input materials. In some examples, the system can receive information specific to packaging sites, e.g., confirm units of measurement, to avoid issues down the road. In one or more examples, the system can use third party software (e.g., J. D. Edwards Enterprise One, version 9.1) to obtain the inventory information to complete the bill of materials. In one or more examples, the system can receive reconciliation limits 134 for the output materials at Block 162. For example, the reconciliation limits 134 may be obtained from the master data 102 or manually input by a user.

In one or more examples, the system may obtain an approval of the bill of materials 112. In one or more examples, the approval may be provided by one or more designated production users. For example, the system may cause a display to present the completed bill of materials 112, via Production Facilitator UI 120, for review by the designated production facilitator user. The system can then receive an approval by receiving the credentials (e.g., username and password) of a production approver. In one or more examples, the system can cause a notification to be sent to a production approver that the bill of materials 112 are ready for review and approval. In one or more examples, the process 150 may not progress to Block 164 without approval of the bill of materials 112 and the study instructions 110.

At Block 164, the system can generate a lot assignment, e.g., lot assignment 114. For example, the Production Facilitator UI 120 can provide a user interface where a production user can enter and/or update information associated with the lot assignment. In one or more examples, the lot assignment can be used to associate a batch record with a particular packaging site. For example, the study batch record 108, e.g., the bill of materials 112, may indicate the output material(s), e.g., a 60-count bottle including 60 tablets, while the lot assignment 114 may specify that 100 of these bottles should be included in a particular batch at a particular packaging site. In one or more examples, the Production Facilitator UI 120 present a list of the approved bill of materials 112 (e.g., each bill of materials corresponding to a respective study part) to a production user. The Production Facilitator UI 120 can then receive an indication from the user indicating which bills of materials should be included in the lot assignment and the packaging site(s) where the output materials are to be packaged. In this manner, the system can determine the output materials and the quantity of these materials to be produced and where the output materials are to be produced.

In one or more examples, the system can receive an indication to include sample output materials and/or retainer output materials. The retainer output materials may correspond to output materials that should be retained for regulatory inspections. The number of sample output materials and retainer output materials can be added to the quantity of output materials to be produced in the lot assignment. For example, if the quantity of output materials for the clinical study itself includes 100 bottles, the number of sample output materials is 5 bottles and the number of retainer output materials is 10 bottles, then the total quantity for the lot assignment is 115 bottles.

In one or more examples, the system can access an inventory of the selected packaging sites. In one or more examples, the inventory information can include various input materials and corresponding supplier lot numbers, supplier lot sizes, and supplier lot expiration dates associated with a packaging site. In one or more examples, a user can assign specific supplier lots to an input material included in the selected bill of material. For example, the system can receive an indication of the supplier lot numbers assigned to the input material for the selected bill of materials. In one or more examples, the system can automatically assign supplier lot numbers to an input material based on the total quantity of the input material and one or more of the respective supplier lot size and/or respective supplier lot expiration date. In this manner, the system can ensure that there is sufficient inventory at a particular site to package the output materials associated with the batch records, prior to the batch records being sent to the packaging site.

In one or more examples, the system may prioritize supplier lots with the smallest lot size and the earliest expiration date. For example, if the quantity indicates that 100 60-count bottles including 60 tablets are to be packaged at a packaging site, the system can access the inventory for the packaging site. Continuing with this example, the packaging site may have five supplier lots corresponding to the 60-count bottle input material. According to embodiments of this example, the system can determine a respective supplier lot size and expiration date for each of the supplier lots and assign the supplier lot with the smallest lost size(s) and earliest expiration date(s) to the 100 60-count bottle input material.

In this manner, the system can reduce waste and the amount of human manual labor at the packaging site. For example, typically, a worker at the packaging site would be responsible for reviewing a study batch order, and manually looking up and comparing the quantity of the input materials against the inventory available at the packaging site. Not only does this process require considerable time and effort, but there is no guarantee that the packaging site will have the necessary inventory to fulfill the study batch record. Accordingly, embodiments of the present disclosure improve on the current state of the art by ensuring that there is sufficient inventory prior to sending the study batch records to a packaging site, reducing waste by assigning supplier lots with earlier expiration dates to be used first, and reducing the amount of manual labor associated with confirming the inventory.

In one or more examples, the system may obtain an approval of the lot assignment 114. For example, the system may cause a display to present the completed lot assignment, via Production Facilitator UI 120, for review by a designated production user. The system can then receive an approval by receiving the credentials (e.g., username and password) of a production approver. In one or more examples, the system can cause a notification to be sent to a production approver that the lot assignment is ready for review and approval.

At Block 166, the system can create a preliminary batch record, e.g., corresponding to batch record 116. For example, the Production Facilitator UI 120 can provide a user interface where a production user can update and review information related to a preliminary batch record. As used herein, the term preliminary batch record may refer to a batch record that has not been approved. An executable batch record may refer to a preliminary batch record that has been approved. In one or more examples, the preliminary batch record may be automatically generated upon receiving approval of the lot assignment 114. The preliminary batch record may include the bill of materials 112 selected in the lot assignment at Block 164 and the corresponding packaging instructions 110 for the respective bill of materials 112.

In one or more examples, the system can receive information regarding the equipment, labels, deviations, and change controls at Block 166. For example, the Production Facilitator UI 120 can provide a user interface to receive an indication of which equipment should be used when packaging the batch record. In one or more examples, the equipment information may be obtained at Block 158. As another example, the system can receive an indication of which labels should be used. In one or more examples, specific labels may be produced for specific packaging runs. For example, sequence numbers may be generated ensure proper randomization of the output materials, e.g., bottles and/or kits. As another example, deviations (e.g., reasons for deviating from the packaging instructions) can be included in the preliminary batch record to provide operators at the packaging site an explanation as to why they may need to deviate from the packaging instruction. For example, a deviation may occur if it is discovered that the seals on the bottles are faulty. The deviations included in the batch record can provide an explanation as to why the operators should de-bottle the batch.

In one or more examples, the system may obtain an approval of the preliminary batch record 116 by a production user. For example, the system may cause a display to present the completed preliminary batch record, via Production Facilitator UI 120, for review by the designated production user. The system can then receive an approval by receiving the credentials (e.g., username and password) of a production approver. In one or more examples, the system can cause a notification to be sent to an approver that the batch record 116 is ready for review and approval. In one or more examples, the executable batch record is generated upon receiving a customer approval.

Embodiments of the present disclosure can also provide a system for customers to efficiently produce additional batches of output materials for the same study using one or more components of an executable batch record for the same study. For example, traditionally, customers that would like to produce additional batches of output materials for a study would have to begin the process of generating a batch record for a particular site from the start (e.g., creating study information, defining output materials, input materials, packaging sites, etc.) In contrast, systems according to embodiments of the present disclosure can create new executable batch records by creating a new lot assignment corresponding to a bill of materials for the desired output materials. Thus, the system can create new executable batch records by having production users update the new quantities and inventory numbers. In one or more examples, if the new batch record requires updated packaging instructions, the packaging instructions may be updated accordingly. Thus, embodiments according to the present disclosure can provide a centralized and efficient way to produce output materials for clinical trial studies without creating a new study batch record for each executable batch record.

FIG. 1D illustrates an exemplary process 160 for generating an executable batch record for packaging materials for a clinical trial study, in accordance with some embodiments. Process 160 is performed, for example, using one or more electronic devices implementing a software platform. In some examples, process 160 is performed using a client-server system, and the blocks of process 160 are divided up in any manner between the server and a client device. In other examples, the blocks of process 160 are divided up between the server and multiple client devices. In other examples, process 160 is performed using only a client device or only multiple client devices. In process 160, some blocks are, optionally, combined, the order of some blocks is, optionally, changed, and some blocks are, optionally, omitted. In some examples, additional steps may be performed in combination with the process 160. Accordingly, the operations as illustrated (and described in greater detail below) are exemplary by nature and, as such, should not be viewed as limiting.

In one or more examples, the process 160 can correspond to a process associated with the Production Facilitator UI 120 and the Customer UI 180. For example, process 160 can correspond to a process for generating an executable batch record that includes receiving approval from one or more customer approvers. In one or more examples, one or more blocks of process 160 can correspond to blocks associated with process 150.

At Block 152, the system can the system can access master data, e.g., master data 102, associated with a customer planning to conduct a clinical trial study, as discussed above with respect to process 150. At Block 154, the system can create study information, e.g., study information 104. For example, the system can obtain study information corresponding to a clinical trial study to be run by the customer, as discussed above with respect to process 150. At Block 156, the system can obtain study part data, e.g., study part 106, as discussed above with respect to process 150. At Block 158, the system can create a study batch record, e.g., study batch record 108, as discussed above with respect to process 150.

At Block 160, the system can obtain study instructions associated with the study batch record, e.g., study instructions 110 associated with study batch record 108, as discussed above with respect to process 150. At Block 182, the system can obtain customer approval for the study instructions 110. In one or more examples, the customer approval may be obtained via Customer UI 180. In one or more examples, Block 182 may be optional based a selection of which components are to be submitted for customer approval made at Block 154. For example, if the system did not receive an indication to obtain customer approval for the study instructions, then the system may forgo Block 182. In one or more examples where Block 182 is implemented, the system cause a display to present the completed study instructions for review by a customer approver. The system can then receive an approval by receiving the credentials (e.g., username and password) of the customer approver as well as the signature of the customer approver. In one or more examples, the approval process for a customer approver may be compliant with relevant regulations. In one or more examples, the system can cause a notification to be sent to the customer approver that the study instructions are ready for review and approval. In one or more examples, the process 160 may not progress to Block 164 without customer approval of the study instructions 110.

At Block 162, the system can obtain the bill of materials associated with the study batch record, e.g., bill of materials 112 associated with the study batch record 108, as discussed above with respect to process 150. At Block 184, the system can obtain customer approval for the bill of materials. In one or more examples, the customer approval may be obtained via Customer UI 180. In one or more examples, Block 184 may be optional based a selection of which components are to be submitted for customer approval made at Block 154. For example, if the system did not receive an indication to obtain customer approval for the bill of materials, then the system may forgo Block 184. In one or more examples where Block 184 is implemented, the system can cause a display to present the completed bill of materials for review by a customer approver. The system can then receive an approval by receiving the credentials (e.g., username and password) of the customer approver as well as the signature of the customer approver. In one or more examples, the system can cause a notification to be sent to the customer approver that the bill of materials are ready for review and approval. In one or more examples, the process 160 may not progress to Block 164 without customer approval of the bill of materials.

At Block 164, the system can generate a lot assignment, e.g., lot assignment 114, as discussed above with respect to process 15. At Block 186, the system can obtain customer approval for the lot assignment. In one or more examples, the customer approval may be obtained via Customer UI 180. In one or more examples, Block 186 may be optional based a selection of which components are to be submitted for customer approval made at Block 154. For example, if the system did not receive an indication to obtain customer approval for the lot assignment, then the system may forgo Block 186. In one or more examples where Block 186 is implemented, the system can cause a display to present the completed lot assignment for review by a customer approver. The system can then receive an approval by receiving the credentials (e.g., username and password) of the customer approver as well as the signature of the customer approver. In one or more examples, the system can cause a notification to be sent to the customer approver that the lot assignment is ready for review and approval. In one or more examples, the process 160 may not progress to Block 166 without customer approval of the lot assignment.

At Block 166, the system can generate a batch record, e.g., batch record 116, as discussed above with respect to process 150. At Block 188, the system can obtain customer approval for the batch record. In one or more examples, the customer approval may be obtained via Customer UI 180. In one or more examples, Block 188 may be optional based a selection of which components are to be submitted for customer approval made at Block 154. For example, if the system did not receive an indication to obtain customer approval for the preliminary batch record, then the system may forgo Block 188. In one or more examples where Block 188 is implemented, the system can cause a display to present the completed preliminary batch record for review by a customer approver. The system can then receive an approval by receiving the credentials (e.g., username and password) of the customer approver as well as the signature of the customer approver. In one or more examples, the system can cause a notification to be sent to the customer approver that the batch record is ready for review and approval. In one or more examples, the process 160 may not generate a completed executable batch record without customer approval of the batch record.

### Master Data

The master data can be used to create and maintain information associated with one or more customers. The master data, e.g., master data 102, can include customer information 122, contacts 124, packaging sites 126, packaging instructions 128, instruction sets 130, equipment 132, reconciliation limits 134, and studies 136. In one or more examples, the master data can be configured to interface with one or more third-party software and/or servers. For example, in one or more examples, the master data can access information associated with a customer in J. D. Edwards EnterpriseOne.

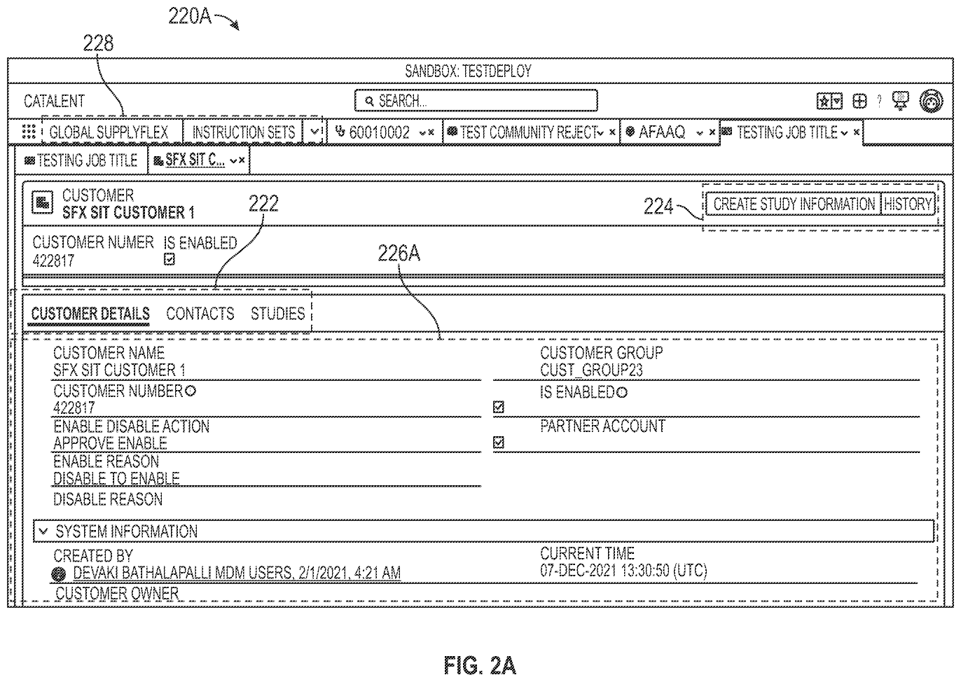

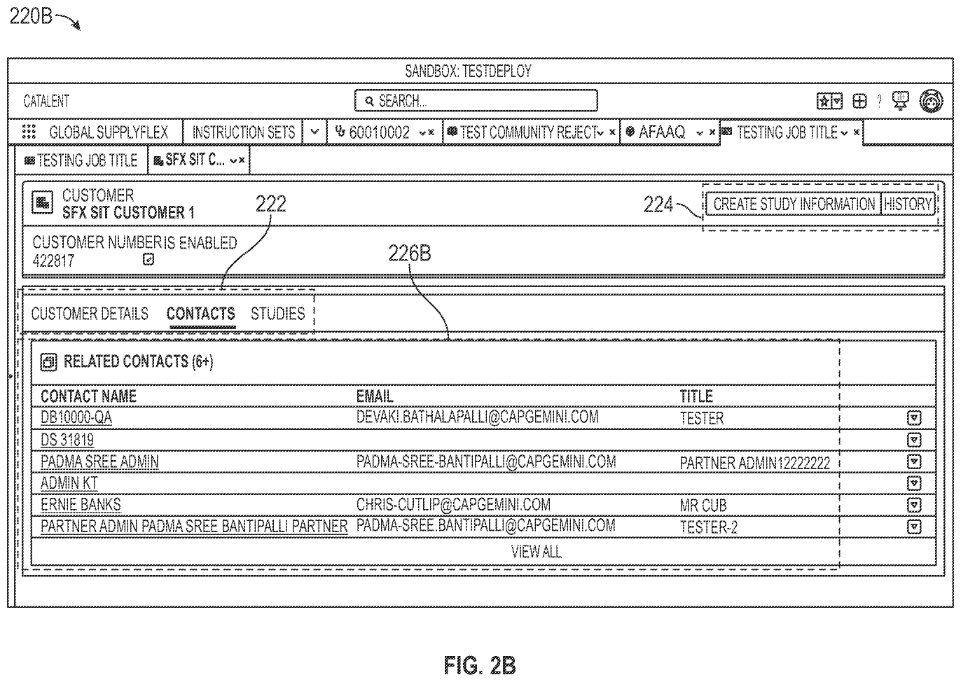

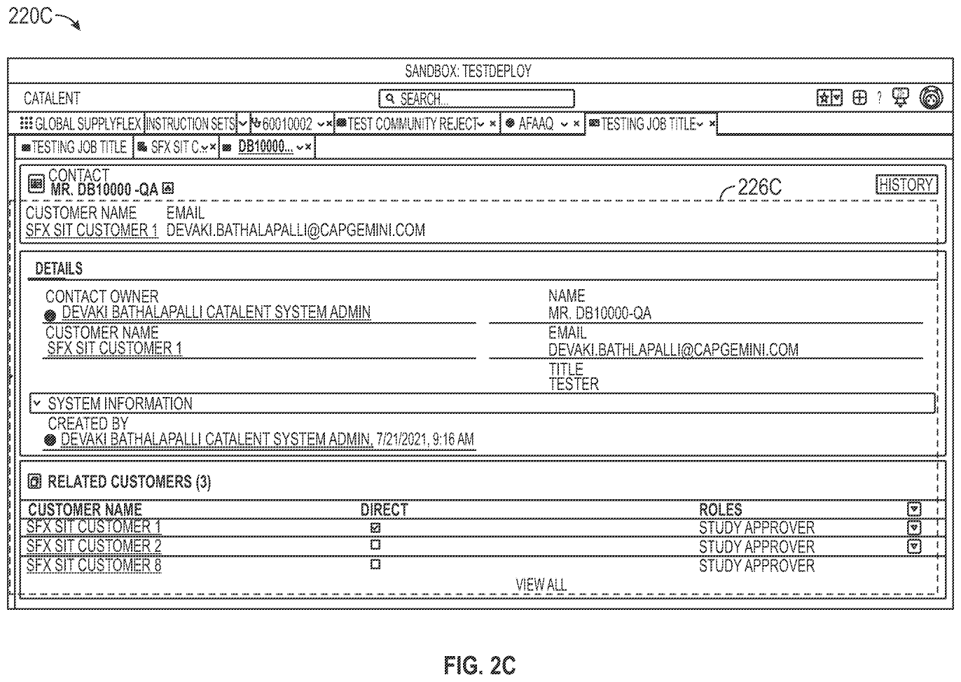

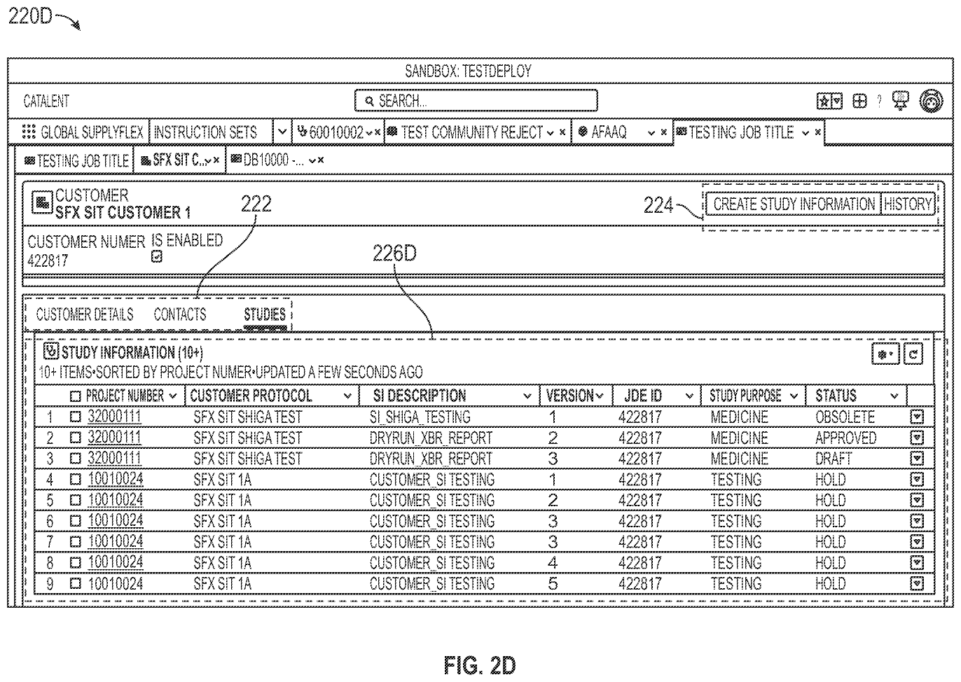

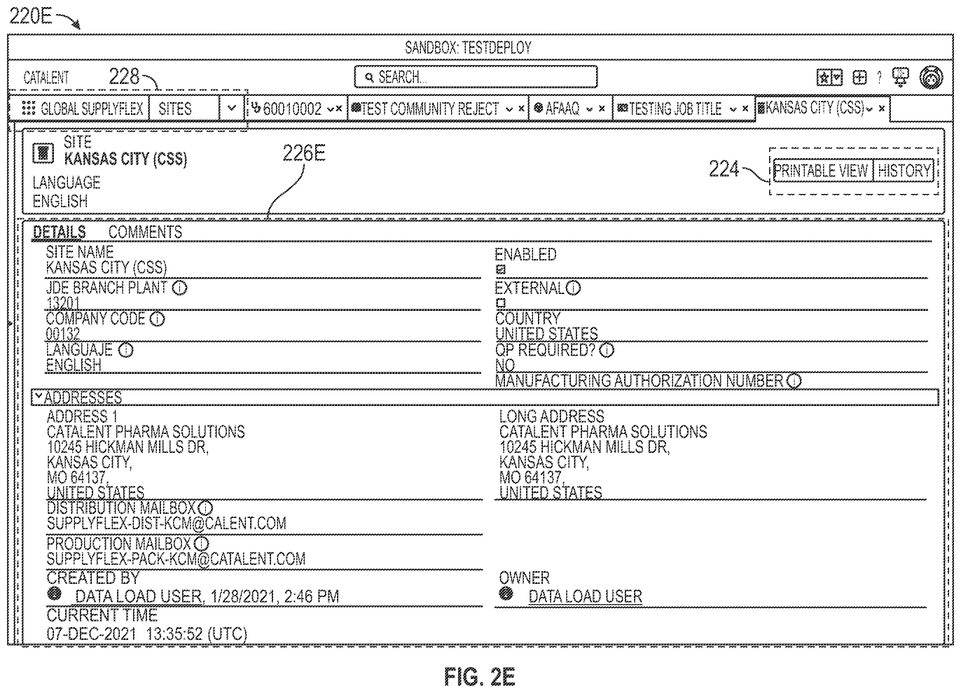

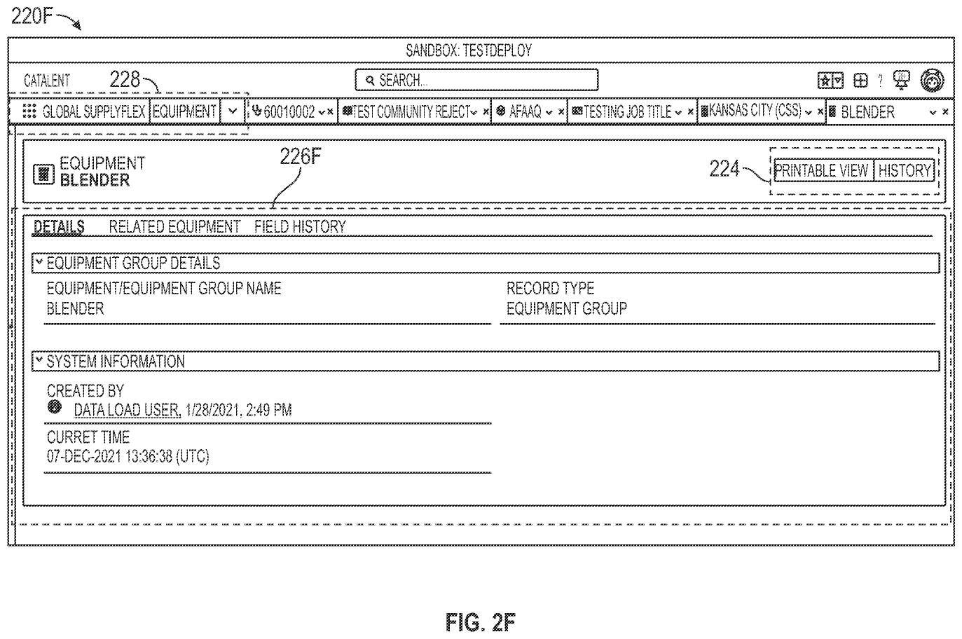

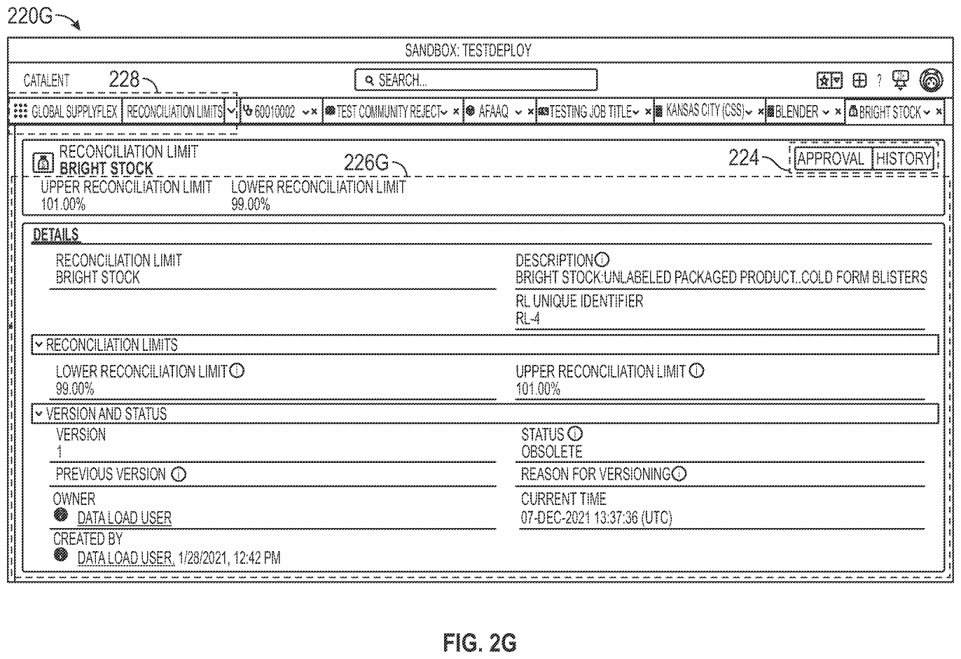

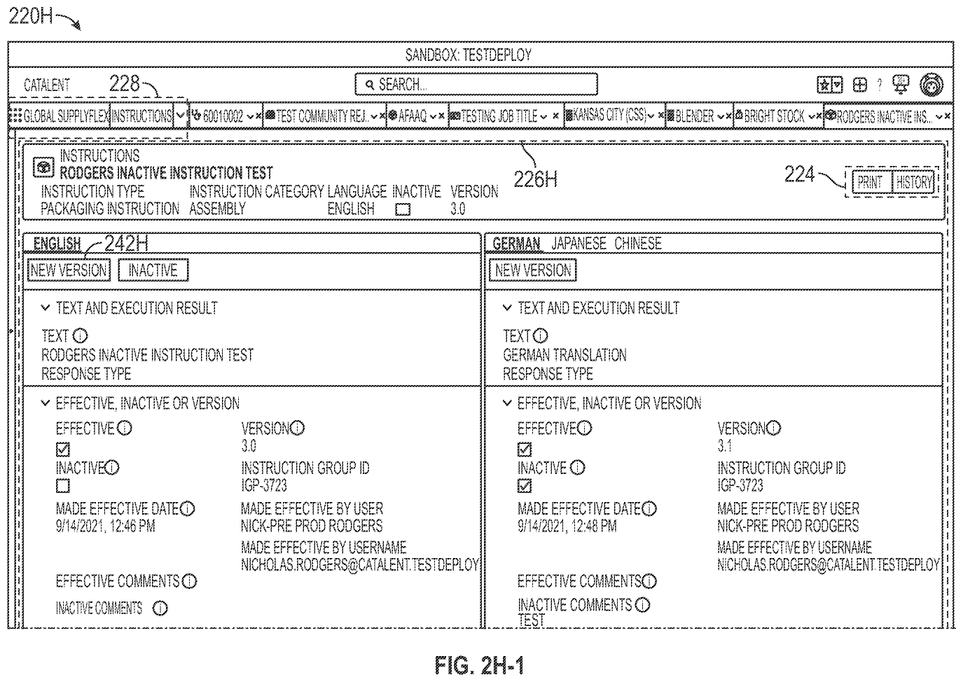

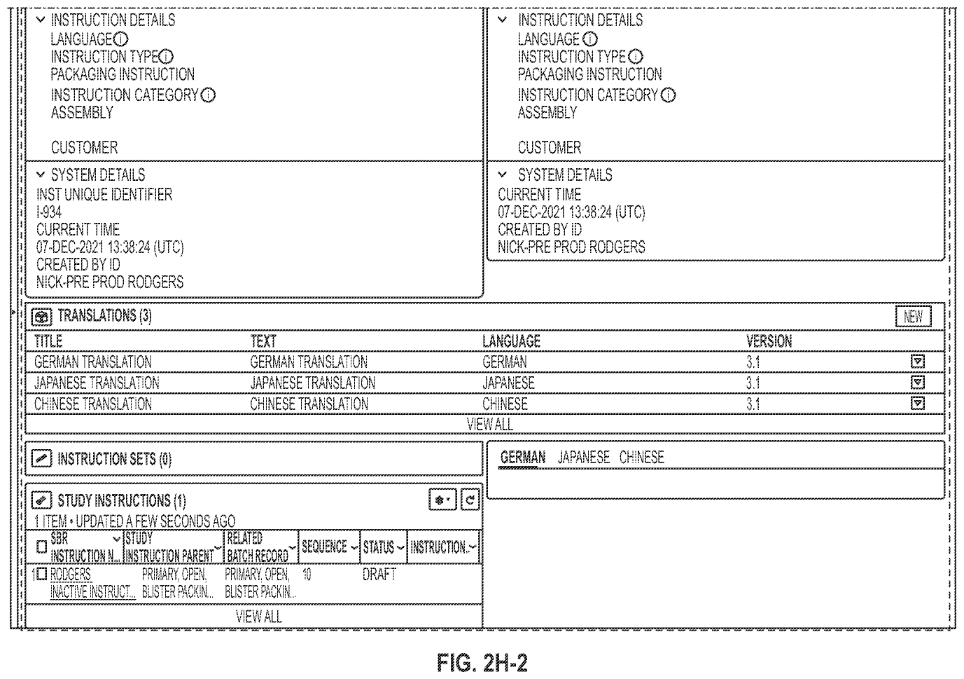

FIG. 2A illustrates an exemplary Customer Details page 220A associated with the Master Data User Interface (UI) and presented via the Production Facilitator UI 120. As shown in the figure, the Customer Details page 220A can include a plurality of navigational user affordances 222, a plurality of operational user affordances 224, and a Customer Details information display region 226A. In one or more examples, the Customer Details information display region 226A can correspond to an area of the Customer Details page 220A that displays information regarding the selected customer. As shown in the figure, the Customer Details information display region 226A can display information included, but not limited to, a customer name, a customer number, a customer group, an enable/disable information, system information, and the like.

In one or more examples, the Master Data UI navigational user affordances 222 can include a plurality of user affordances, where each user affordance corresponds to a tab associated with the Master Data UI and a corresponding page. As shown in the figure, the navigational user affordances 222 can include a Customer Details tab, a Contacts tab and a Studies tab. The examples of navigational user affordances 222 are merely exemplary and more or less navigational user affordances can be used without departing from the scope of this disclosure. A user can navigate between the respective pages associated with the tabs by selecting on the corresponding user affordance.

In one or more examples, the system can also navigate between pages and/or study components using navigational bar 228. For example, the system can receive a selection of a page of any of the study components (e.g., master data, study information, study part, study batch record, packaging instructions, bill of materials, lot assignment, batch record) from the drop down menu of the navigational bar 228, and subsequently present the selected page.

In one or more examples, the plurality of operational user affordances 224 can include a plurality of buttons that each correspond to a different action that can be implemented by a user. As shown in the figure, the operational user affordances 224 can include, but is not limited to a create study information user affordance and a history user affordance. In one or more examples, the create study information user affordance can be used to create a study information associated with the customer. In one or more examples, the history user affordance can present an auditing history associated with the customer, master data, and/or other study components.

FIG. 2B illustrates an exemplary Contacts page 220B associated with the Master Data UI and presented via the Production Facilitator UI. As shown in the figure, the Contacts page 220B can include the plurality of navigational user affordances 222, the plurality of operational user affordances 224, and a Contacts information display region 226B. In one or more examples, the Contacts information display region 226B can correspond to an area of the Master Data UI that displays information regarding the contact information for one or more individuals associated with the customer information. For example, as shown in the figure, the contacts can include but is not limited to the name, email, phone number, and title (e.g., position at the company corresponding to the customer), and roles (e.g., role within the system and/or study) associated with the contact.

FIG. 2C illustrates an exemplary Contacts Detail page 220C associated with the Master Data UI and presented via the Production Facilitator UI 120. In one or more examples, the system can receive an indication that a user has selected one of the contacts listed on Contacts page 220B. For example, the user can click on a link associated with one of the listed contacts. The Contacts Detail page 220C can include the information associated with the selected contact presented in the Contacts Detail information display region 226C. As shown in the Figure, the Contacts Detail information display region 226C can include, but is not limited to, a customer name, a customer email, a contact owner, a title, system information, and related customer information, a role for each of the related customers.

FIG. 2D illustrates an exemplary Studies page 220D associated with the Master Data UI and presented via the Production Facilitator UI 120. As shown in the figure, the Studies page 220D can include the plurality of navigational user affordances 222, the plurality of operational user affordances 224, and a Studies information display region 226D. In one or more examples, the Studies information display region 226D can correspond to an area of the Master Data UI that displays a list of the studies associated with the customer. Each study in the list can include, but is not limited to, a project information, a customer protocol, a study information description, a version number, a study purpose, a status, and an external software/server integration number.

FIG. 2E illustrates an exemplary Packaging Site page 220E associated with the Master Data UI and presented via the Production Facilitator UI. In one or more examples, the Packaging Site page 220E can be associated with a specific customer. In one or more examples, the system can navigate to the Packaging Site page 220E via the navigational bar 228. As shown in the figure, the Packaging Site page 220E can include the plurality of operational user affordances 224, and a Packaging Site information display region 226E. In one or more examples, the Packaging Site information display region 226E can correspond to an area of the Master Data UI that displays information associated with the packaging sites associated with the customer. The packaging site information can include, but is not limited to a site name, a site location (e.g., city and country), a company code, a language, one or more addresses, one or more emails, and the like.

FIG. 2F illustrates an exemplary Equipment page 220F associated with the Master Data UI and presented via the Production Facilitator UI 120. The equipment described on the equipment page may correspond to equipment used to package one or more output materials for a study. In one or more examples, the equipment may be associated with a specific customer. In one or more examples, the system can navigate to the Packaging site page 220E via the navigational bar 228. As shown in the figure, the Equipment page 220F can include the plurality of operational user affordances 224, and an Equipment information display region 226F. In one or more examples, the Equipment information display region 226F can correspond to an area of the Master Data UI that displays information associated with the equipment associated with the customer. The equipment information can include, but is not limited to one or more equipment types, equipment group details, a record type, system information, and the like.

FIG. 2G illustrates an exemplary Reconciliation Limits page 220G associated with the Master Data UI and presented via the Production Facilitator UI 120. The reconciliation limits relate to permissible deviations in an output material from an output material packed according to the correct packaging instructions. In one or more examples, reconciliation limits can be specific to a customer. For example, a 100% reconciliation limit for a 60-count bottle of tablets would have to include 60 tablets, while the same output material (e.g., 60-count bottle) with a lower reconciliation limit of 98% and an upper reconciliation limit of 103% could include 59-61 tablets. In one or more examples, the system can navigate to the Reconciliation Limits page 220G via the navigational bar 228. As shown in the figure, the Reconciliation Limits page 220G can include the plurality of operational user affordances 224, and a Reconciliation Limit information display region 226G. In one or more examples, the Reconciliation Limit information display region 226G can correspond to an area of the Master Data UI that displays information associated with the reconciliation limits associated with the customer. The reconciliation limits can include a lower reconciliation limit, an upper reconciliation limit, a reconciliation identifier, a reconciliation limit description, a version, a status, and the like. The upper and lower reconciliation limits can be provided as a percentage. In one or more examples, the operation user affordances can include an approval button (e.g., for approving reconciliation limits) and a history button (e.g., for reviewing an audit history associated with the reconciliation limits).

FIGS. 2H-1 and 2H-2 illustrate an exemplary Packaging Instructions page 220H associated with the Master Data UI and presented via the Production Facilitator UI 120. The packaging instructions can be used by an operator at one or more packaging sites to package the output materials for a study. In one or more examples, the system can navigate to the Packaging Instructions page 220H via the navigational bar 228. As shown in the figure, the Packaging Instructions page 220H can include the plurality of operational user affordances 224, and a Packaging Instructions information display region 226H. In one or more examples, the Packaging Instructions information display region 226H can include, but is not limited to, a list of translations, a list of related instruction sets, and for each language, a text and execution result, an indication of whether the instructions are effective or inactive, a version number, an instruction category, an instruction type (e.g., packaging instructions, in process checklist, or important notes), an instruction identifier, an effective date, comments, an instruction group ID, instruction details, a language of the instructions, available translations of the instructions. In one or more examples, when an instruction is marked effective, the instruction can be used an instruction set, e.g., instruction set 130. In one or more examples, effective instructions cannot be directly edited. Rather, in order to edit instructions marked effective, a new version of the instruction can be created, e.g., using the “New Version” user affordance 242H.

FIG. 2I illustrates an exemplary Instruction Sets page 220I associated with the Master Data UI and presented via the Production Facilitator UI. The instruction sets can include one or more effective instructions. In one or more examples, the system can navigate to the Instruction Sets page 220I via the navigational bar 228. As shown in the figure, the Instruction Sets page 220I can include an Instructions information display region 226I. In one or more examples, the Instructions information display region 226I can include, but is not limited to, an instruction type (e.g., packaging instructions, in check process, important notes), an instruction status, a version, an instruction category, an indication of whether the instructions are effective, a search bar, one or more languages, a text, a version, an indication of whether the instruction set is inactive, and instruction grouping information (e.g., name, description, and language).

### Study Information

The study information (e.g., study information 104) can correspond to information associated with a clinical trial study to be run by a customer company. A production user can create and input the study information associated with a study (e.g., Study X) via the Production Facilitator UI. For example, a customer intending to run a study that includes one or more pharmaceutical products provided to study participants. A production user can create a study information in the system corresponding to the customer to generate an executable batch record that can be used to package the pharmaceutical products provided to the study participants.

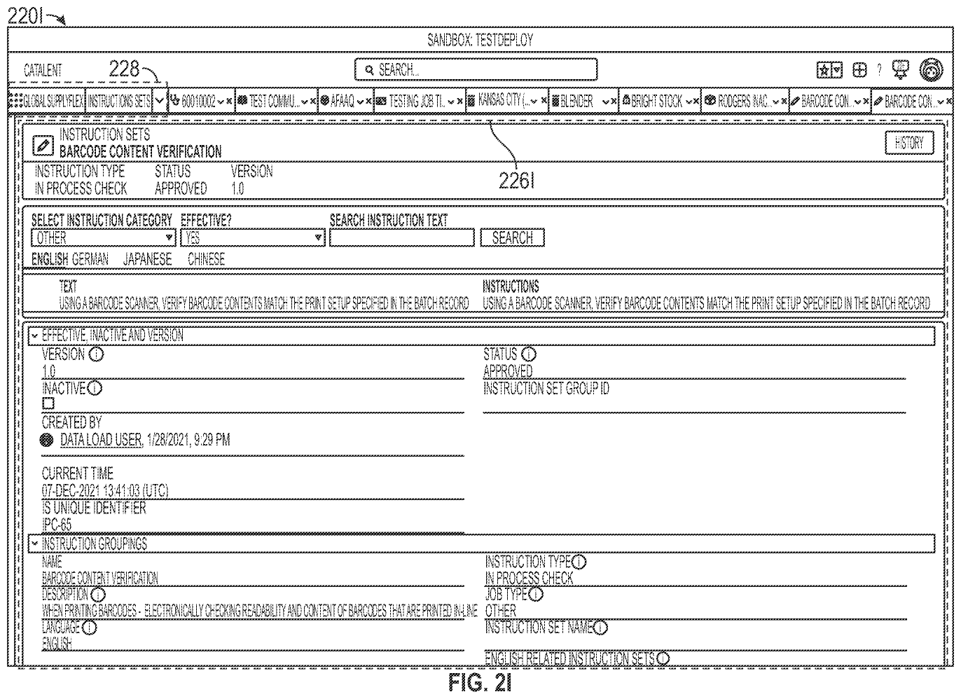

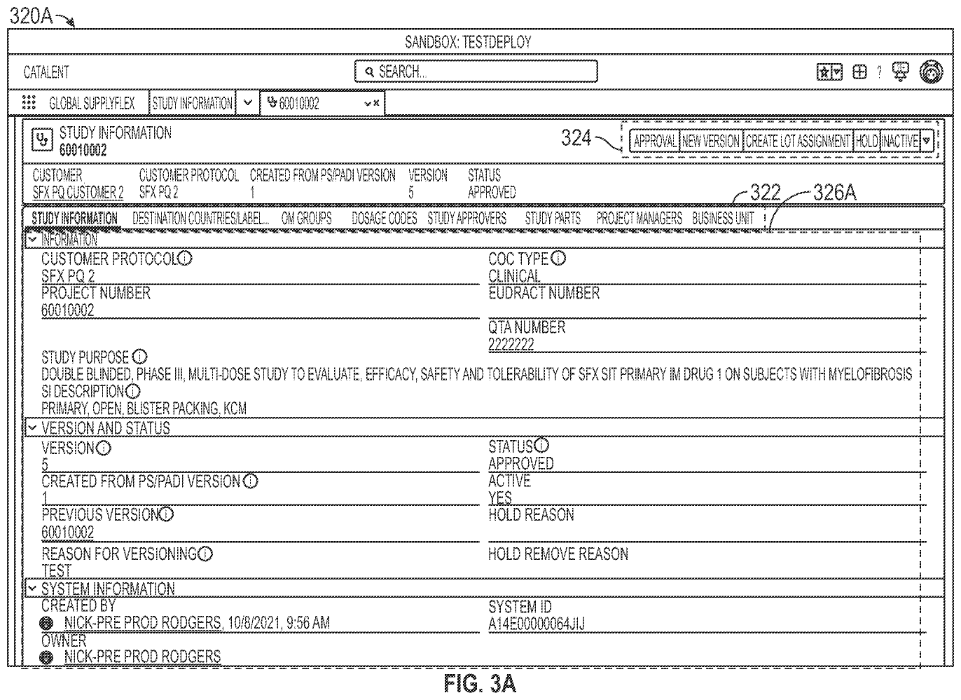

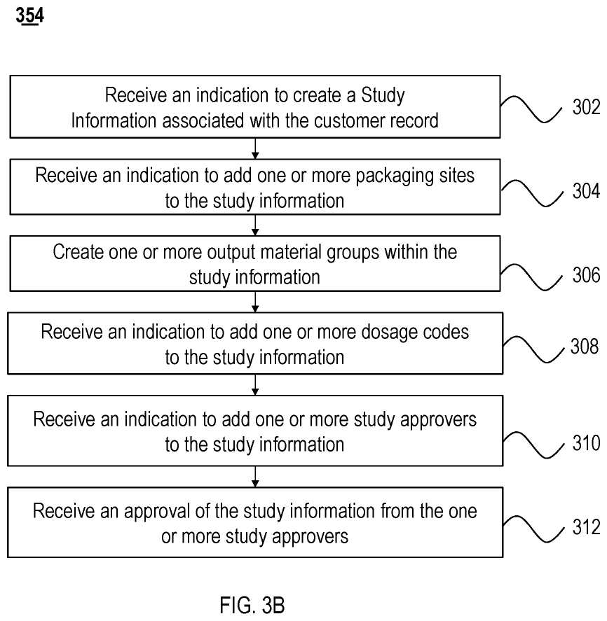

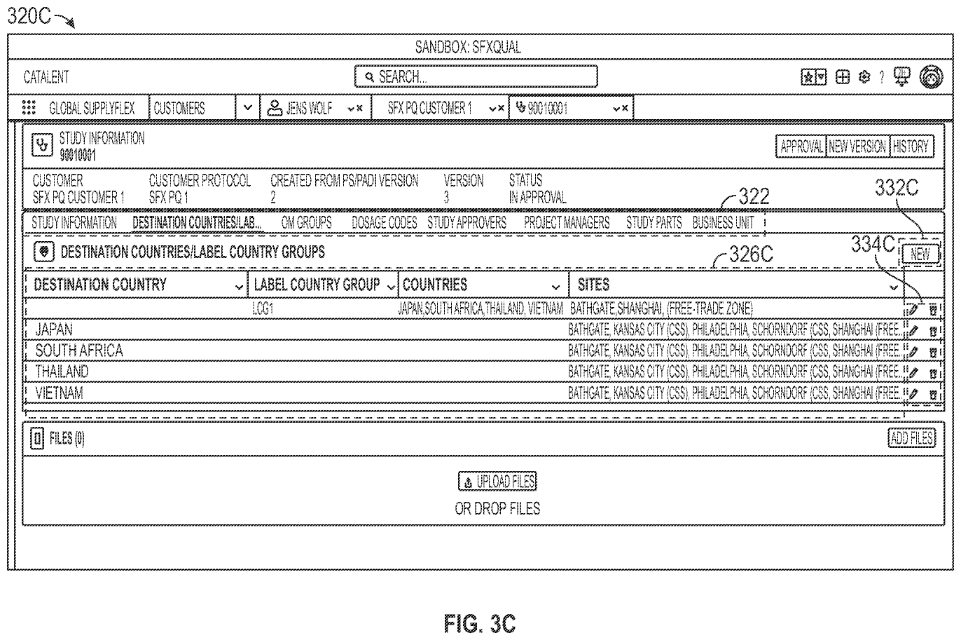

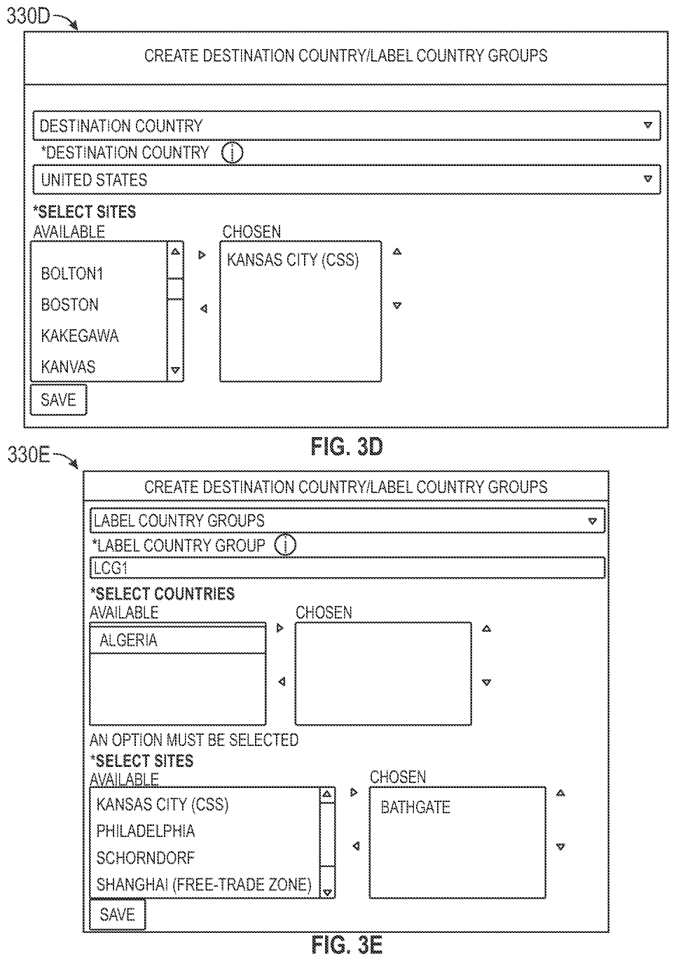

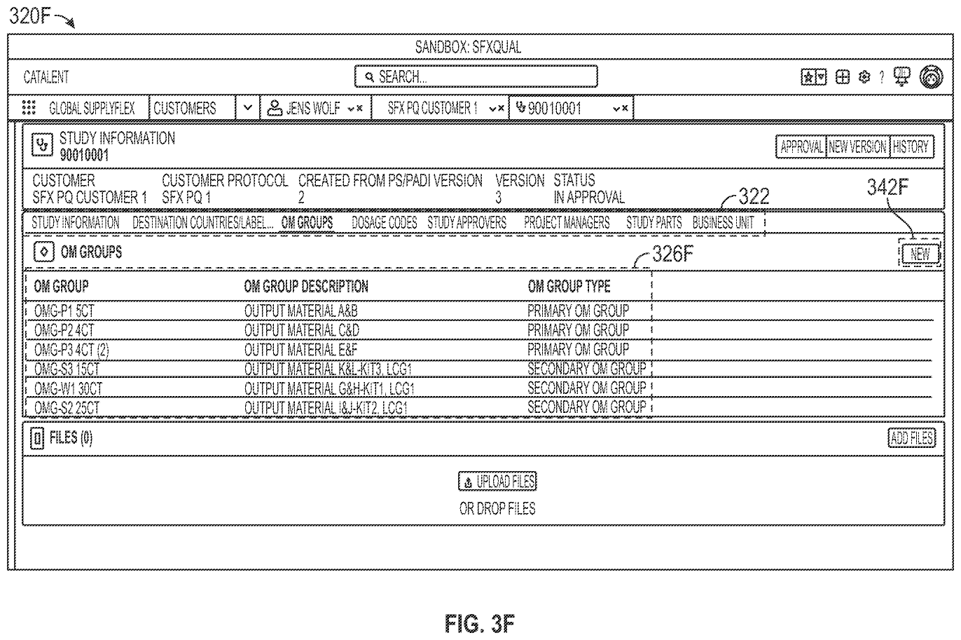

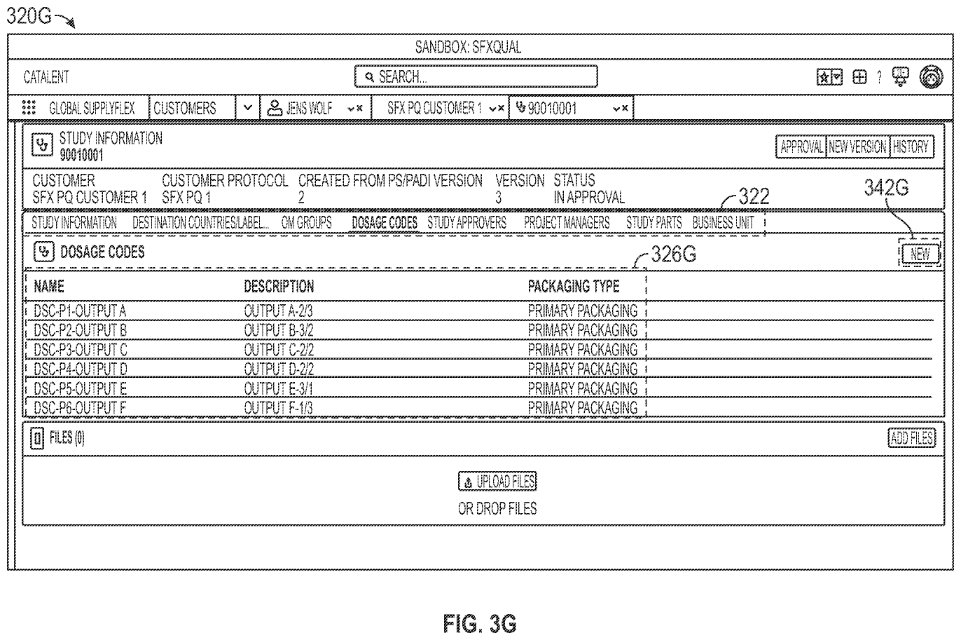

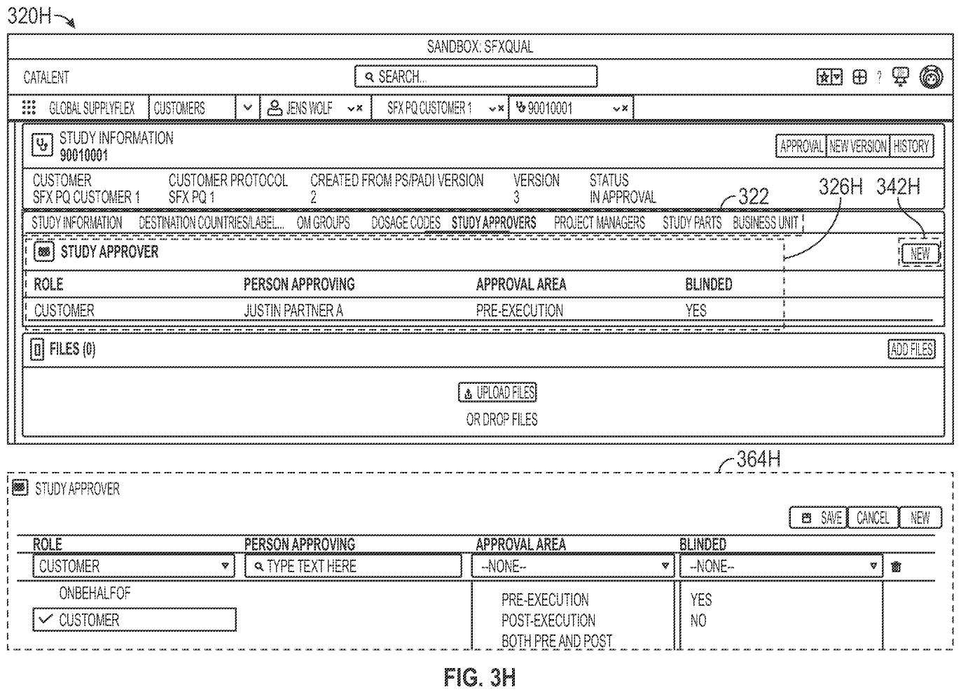

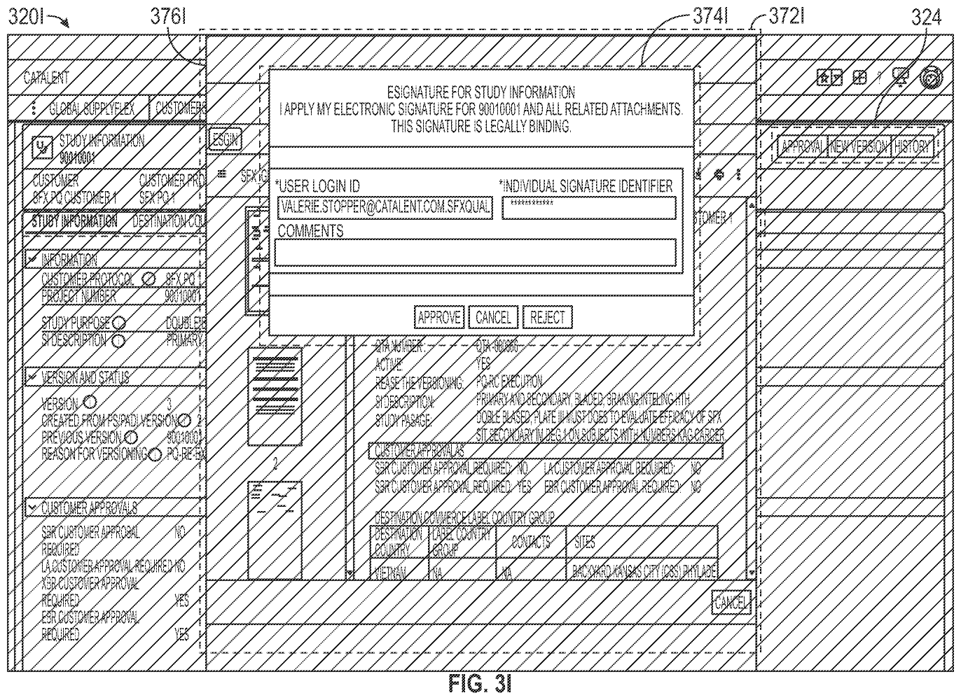

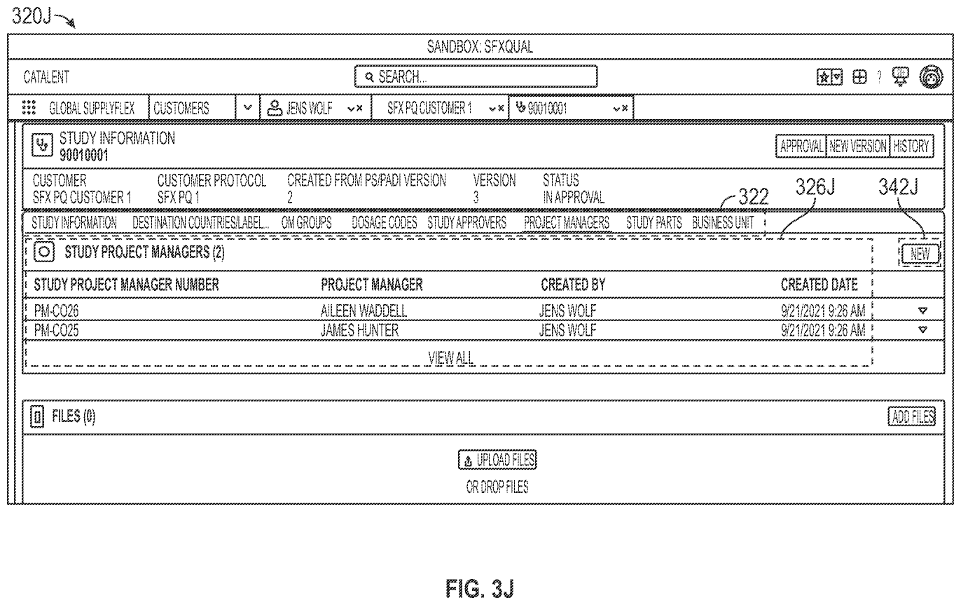

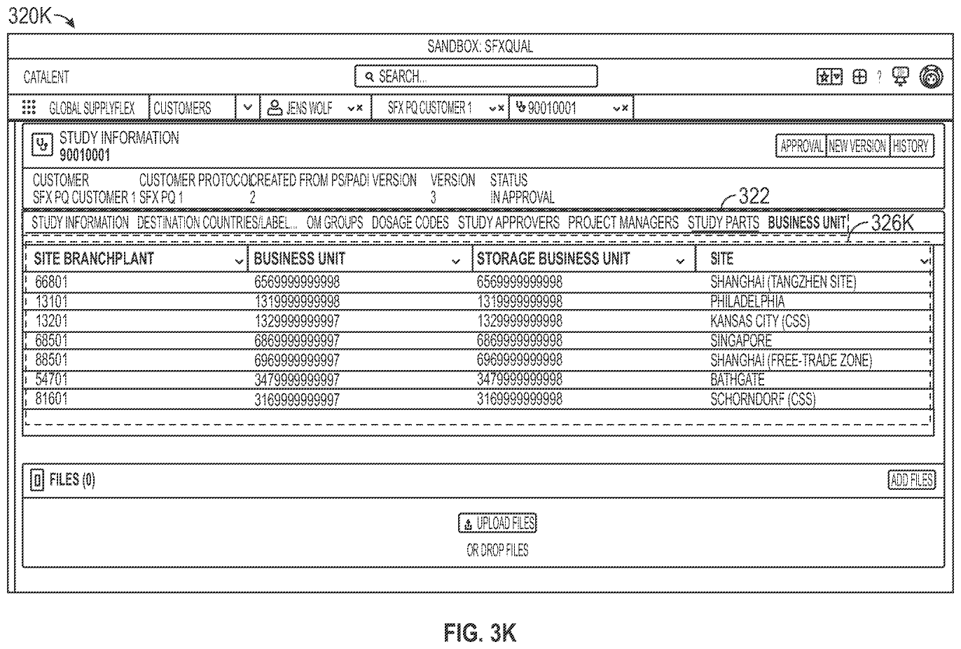

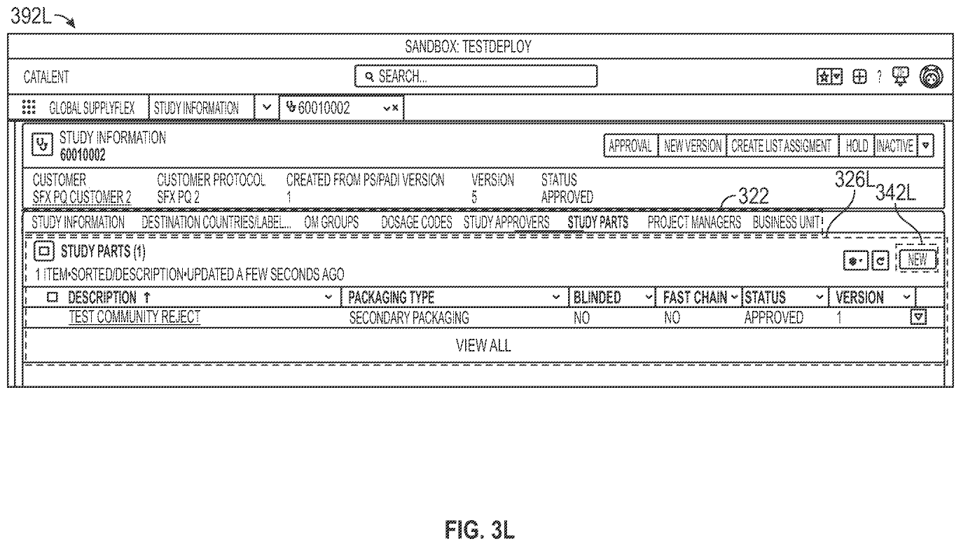

In one or more embodiments, the system can include a Study Information UI. The Study Information UI can be presented via the Production Facilitator UI 120 for creating, reviewing, and obtaining information related to the studying information. FIG. 3A illustrates an exemplary Study Information page 320A associated with the Study Information UI in accordance with embodiments of the present disclosure. As shown in the figure, the Study Information page 320A can include a plurality of navigational user affordances 322, a plurality of operational user affordances 324, Study Information display region 326A.

In one or more examples, the Study Information display region 326A can correspond to an area of the study information UI that displays information related to the study information. As shown in the figure, the Study Information display region 326A can display information included, but not limited to, a customer protocol, a customer number a CoC type, a study purpose, a study information description, the version of the study information, a status of the version, a reason or the version, the creator of the current version of the study information, and the owner of the study information.

In one or more examples, the navigational user affordances 322 can include a plurality of user affordances, where each user affordance corresponds to a tab and corresponding page associated with the study information. As shown in the figure, the navigational user affordances 322 can include a Study Information tab, a Destination Countries/Label Country Groups tab, an Output Material (OM) groups tab, a Dosage Codes tab, a Study Approvers tab, a Study Parts tab, a Project Managers tab, and a Business Unit tab. The examples of navigational user affordances 322 are merely exemplary and more or less navigational user affordances can be used without departing from the scope of this disclosure.

In one or more examples, the Production Facilitator UI 120 can receive an indication that a user has selected a navigation user affordance. Upon receiving the indication, the Production Facilitator UI 120 can navigate to a page corresponding to the selected study navigation user affordance 322. For example, if the system receives an indication that a user has selected the OM groups tab, the system can update the Production Facilitator UI 120 to display the page associated with the OM groups tab. In one or more examples, the system can obtain data regarding each of the navigational user affordances 322 via the corresponding page associated with the respective navigation user affordance. For example, the system can obtain information related to the OM groups via the page associated with the OM groups tab.

In one or more examples, the plurality of operational user affordances 324 can include a plurality of buttons that each correspond to a different action that can be implemented by a user. As shown in the figure, the operational user affordances 324 can include, but is not limited to an approval button, a new version button, a create lot assignment button, a hold button, an inactive button, and the like.

In one or more examples, the approval button can be used to receive an approval of the study information by a production user. In one or more examples, a new version button can be used to create a new version of the study information by a production user. In one or more examples, a create lot assignment button can be used to create a lot assignment. In one or more examples, the hold button can be used to change the status of a study information to “hold.” In one or more embodiments, depending on the status of different items associated with study information 104, one or more of the operational user affordances 324 may be inactive and/or one or more of the operational user affordances may be removed or made visible. For example, as will be explained in greater detail below, the create lot assignment button may be inactive and/or not visible, if the system has not received an approval of study instructions 110 and bill of materials 112.

In one or more examples, the Study Information UI of Production Facilitator UI can be configured to obtain information related to study information 104 as discussed above with respect to Block 154 in process 150 and process 160.

FIG. 3B illustrates process 354 for obtaining study information, according to one or more embodiments of the present disclosure. In one or more embodiments, process 354 can correspond to Block 154 discussed above with respect to process 150 and process 160. Process 354 can be performed, for example, using one or more electronic devices implementing a software platform. In some examples, process 354 is performed using a client-server system, and the blocks of process 354 are divided up in any manner between the server and a client device. In other examples, the blocks of process 354 are divided up between the server and multiple client devices. In other examples, process 354 is performed using only a client device or only multiple client devices. In process 354, some blocks are, optionally, combined, the order of some blocks is, optionally, changed, and some blocks are, optionally, omitted. In some examples, additional steps may be performed in combination with the process 354. Accordingly, the operations as illustrated (and described in greater detail below) are exemplary by nature and, as such, should not be viewed as limiting.

At Block 302, the system can receive an indication to create a study information. For example, the system can receive from a production user a search for a particular customer record. The customer record can correspond to customer information 122 from master data 102. In one or more examples, the production user can navigate to the master data UI, e.g., master data UI 220A from FIG. 2A, and use the Create Study Information operational user affordance 222 to initiate the process of creating a new study information associated with a customer in the system. Based on information regarding the customer from the master data 102 and one or more third party software applications (e.g., J. D. Edwards), the system can automatically associate data related to the customer with the study information 104.

In one or more examples, the Production Facilitator UI can receive study information from a production user including, but not limited to, a protocol number, project number, a study purpose, a study information description, and CoC type. In one or more examples, the system may prompt the user to indicate which portions of the study information will require an approval from a customer approver. In one or more examples, the system can be configured to receive a customer approval for one or more of the study instructions 110, bill of materials 112, lot assignment 114, and batch record 116.

Referring to FIG. 3B, at Block 304, the system can receive an indication to add one or more packaging sites to the study information. For example, the Production Facilitator UI 120 can receive an indication that a production user has selected the Destination Countries/Label Country Groups tab from the Study Information navigational user affordances 322. Upon receiving the selection of the Destination Countries/Label Country Groups tab, the system can navigate to the Destination Countries/Label Country Groups page 320C, as shown in FIG. 3C. The Destination Countries/Label Country Groups page 320C can be presented via the Production Facilitator UI 120. As shown in the figure, the Destination Countries/Label Country Groups page 320C can include the plurality of navigational user affordances 322, a new-input user affordance 332C, a Destination Country Information Display region 326C, and a plurality of editing user affordances 334C.

In one or more examples, the system can receive an indication that a production user has selected the new-input user affordance 332C. The new-input user affordance 332C can be used to add a new packaging site to the study information. Upon receiving a selection of the new-input user affordance 332C, the system can cause a Destination Country Information Input Window to be displayed. An exemplary Destination Country Information Input Window 330D is shown in FIG. 3D. As shown in the figure, a production user can select a destination country. Based on the selected destination country, the system can populate one or more available packaging sites located in the selected destination country. The system can receive a selection of one or more packaging sites by the production user. In one or more examples, the system can pre-populate the destination countries, and packaging sites based on packaging sites 126 and/or data obtained from third party software.

In one or more examples, the system can allow a user to bundle one or more packaging sites into a label group. FIG. 3E shows an exemplary Destination Country Group Window 330E. The Destination Country Group Window 330E can be used to group one or more packaging sites into a group. For example, the system can receive a user selection of a “Label Country Groups” from a dropdown menu, a corresponding name for the label group, and one or more packaging sites to be included in the label group. In one or more examples, once a packing site is included in a label group, it cannot be included in a second label group. In one or more examples, a packaging site can be included in any number of label groups.

The Destination Country Information Display region 326C, can display destination countries, packaging sites, and label country groups based on the selections made in the Destination Country Group Window 330D, 330E. A user can edit and/or remove a site from the list shown in Destination Country Information Display region 326C using a corresponding edit user affordance 334C.

At Block 306, the system can receive an indication from a production user to create one or more output material (OM) groups within the study information 104. In one or more examples, Block 306 can be omitted. The OM groups may include one or more output materials that can be grouped together for delivery to a participant in a study. In one or more examples, the system can receive an indication that a user has selected the OM Groups tab from the study information navigational user affordances 322. Upon receiving the selection of the OM Groups tab, the system can navigate to the OM Groups page 320F, as shown in FIG. 3F. OM Groups page 320F can be presented via Production Facilitator UI 120. As shown in the figure, the OM Groups page 320F can include the plurality of navigational user affordances 322, a new-input user affordance 342F, and an OM Group Information Display region 326F. The plurality of navigational user affordances 322 may be similar to the plurality of navigational user affordances 322 described above. The new OM group user affordance 342F can be selected by a user to create a new OM group. The OM Group Information Display region 326F can display a list including the existing OM groups that have been created.

At Block 308, the system can receive an indication to add one or more dosage codes to the study information. The dosage codes can be assigned to the output materials to indicate the amount of each drug that goes into a respective output material. In one or more examples, Block 308 can be omitted. In one or more examples, the system can receive an indication that a user has selected the Dosage Codes tab from the study information navigational user affordances 322. Upon receiving the selection of the Dosage Codes tab, the system can navigate to the Dosage Codes page 320G, as shown in FIG. 3G. The Dosage Codes page 320G can be presented via Production Facilitator UI 120. As shown in the figure, the Dosage Codes page 320G can include the plurality of navigational user affordances 322, a new-input user affordance 342G, and a Dosage Codes Information Display region 326F. The plurality of navigational user affordances 322 may be similar to the plurality of navigational user affordances 322 described, above. The new-input user affordance 342G can be selected by a user to create a new OM group. The Dosage Codes Information Display region 326G can display the dosage codes for the various output materials associated with the study information.

At Block 310, the system can receive an indication to add one or more study approvers to the study information. The approvers can correspond to customer users authorized to approve one or more study components. The customer approvers can be associated with credentials used to approve one or more components of the study information. As discussed above, the system can receive an indication of which components of the study information should be approved when the study information 104 is being configured at Block 302 and/or Block 154. In one or more examples, the customer approvers may be configured to approve one or more of the study instructions, bill of materials, lot assignment, and batch records.

In one or more examples, the Production Facilitator UI can receive an indication that a production user has selected the Study Approvers tab as provided in the navigational user affordances 322. Upon receiving the selection of the Study Approvers tab, the system can navigate to the Study Approvers page 320H, as shown in FIG. 3H. The Study Approvers page 320H can be presented via Production Facilitator UI 120. As shown in the figure, the Study Approvers page 320H can include the plurality of navigational user affordances 322, a new-input user affordance 342H, and a Study Approvers Information Display region 326H. The plurality of navigational user affordances 322 may be similar to the plurality of navigational user affordances 322 described, above. The new-input user affordance 342H can be selected by a production user to add a new Study Approver. In one or more examples, a drop down menu 364H can be used to add new study approvers upon selecting the new-input user affordance 342H. The Study Approvers Information Display region 326H can display a list of the existing study approvers for the study information.

In one or more examples, the process 354 can include adding one or more project managers to the study. For example, the system can receive an indication from production user to add one or more project managers to the study information. In one or more examples, the system can receive an indication that a production user has selected the Project Managers tab as from the plurality of navigational user affordances 322 in the study information UI 320. Upon receiving the selection of the Project Managers tab, the Production Facilitator UI can navigate to the Project Managers page 320J, as shown in FIG. 3J. As shown in the figure, the Project Managers page 320J can include the plurality of navigational user affordances 322, a new-input user affordance 342J, and a Project Manager information display region 326J. The plurality of navigational user affordances 322 may be similar to the plurality of navigational user affordances 322 described, above. The new-input user affordance 342J can be selected by a production user to add a new Project Manager. The Project Managers Information Display region 326J can display the project managers assigned to the study information.

FIG. 3K illustrates an exemplary Business Unit page 320K. The Business Unit page 320K can be presented via Production Facilitator UI 120. In one or more examples, a user can navigate to a Business Unit page using the study information navigational user affordances 322. In one or more examples, the Business Units can be based on information from the master data 102 and/or obtained from third party software integrated with the system. As shown in the figure, the Business Unit page 390K can include the plurality of navigational user affordances 322 and a Business Unit Information Display region 326K. The plurality of navigational user affordances 322 may be similar to the plurality of navigational user affordances 322 described, above. The Business Unit Information Display region 326K can display a list of the business units associated with various packaging sites associated with the study information.

FIG. 2L illustrates an exemplary Study Parts page 320L. Study Parts page 320L can be presented via Production Facilitator UI 120. In one or more examples, a user can navigate to the Study Parts page 320L using the study information UI navigational user affordances 322. A study part can include one or more output materials for a study that can share the same packaging instructions. As shown in the figure, the Study Parts page 320L can include the plurality of navigational user affordances 322, a new study part user affordance 342L, and a Study Parts Information Display region 326L. The plurality of navigational user affordances 322 may be similar to the plurality of navigational user affordances 322 described, above. The new-input user affordance 342L can be used to add a new study part to the study information 104. In one or more examples, the new study part use affordance 342L may only be active upon receiving an approval of the study information as discussed below (e.g., a production user can add a new study part once the study information is approved). The Study Parts Information Display region 326L can display a list of the study parts 106 associated with the study information 104.

At Block 312, the system can receive an approval of the study information from the one or more production users, e.g., production approvers, as discussed above with respect to process 150 and process 160. In one or more examples, the system may not permit an approval to be accessed without receiving completed destination countries/label groups, OM groups, dosage codes, and customer approvers information. FIG. 3I illustrates an exemplary Approval page 320I presented via Production Facilitator UI 120. The exemplary Approval page 320I can include one or more of an Approval Report 372I and an Approval Window 374I.

In one or more examples, the system can receive an indication that a user has selected the Approval button from the operational user affordances 324. The Approval button can be selected from any of the pages and/or tabs associated with the study information UI. Upon receiving the selection of the Approval button, the system can present an approval report 372I. The approval report can include the study information associated with each of the Information Display regions across the various Study Information tabs. For example, the approval report can include the information included in one or more of the Study Information Display region 326A, the Destination Countries Information Display region 326C, the OM Groups Information Display region 326F, the Dosage Codes Information Display region 326G, the Study Approvers Information Display region 326H, the Project Managers Information Display region 326J, the Business Units Information Display region 326K, or the Study Parts Information Display region 326L.

Once a user is ready to sign the Approval Report 372I, e.g., after the production user has reviewed and verified the information in the Approval Report 372I, the system can receive an indication that the production user has selected the signature button 376I. Upon receiving an input corresponding to the selection of the signature button 376I, the system can cause an Approval Window 374I to appear. As shown in the figure, the signature window can be overlaid on the Approval Report 372I. The signature window 374I can include one or more user affordances for receiving the credentials of the production user. For example, the signature window 374 can include a textbox for a production user to input their user identifier and corresponding password.

The system can then receive an indication of the production user accepting or rejecting the approval. In one or more examples, the production user may cancel the pending approval. Upon receiving an acceptance or rejection of the approval, the system can verify the credentials of the production user. If the credentials are valid and correspond to a designated study approver, then the system can accept or reject the approval based on the indication received from the signature window 374I. If the credentials are valid and do not correspond to a designated study approver, then the system will neither accept nor reject the approval.

Once a study information 102 is approved, the system may not permit changes to be made to the approved version of the study information. If a production user desires to make changes to a study information 102, the production user can create a new version of the study information 102. The system can create a new version of the study information 102 by receiving an input corresponding to a selection of the New Version button from the operational user affordances 324. Changes to the new version of the study information can be made in accordance with the description provided above, e.g., with respect to process 354. Once a new version is created, the previous version will remain the controlling version of the study information until the new version is approved, as described above with respect to Block 312.

### Study Parts

Once a study information 102 is approved, the system can permit a production user to add one or more study parts. The study part can correspond to one or more output materials of a study that can be prepared using the same packaging instructions. In one or more examples, output materials can share the same packaging instructions when the output materials can be packaged under the same conditions. For example, a study part can correspond to a 60-count bottle that is filled with 60 tablets that can be packaged at a controlled room temperature (CRT). In one or more examples, the study part data can include, but is not limited to, output materials, input materials, common materials, drug categorizations, alternative materials, storage temperature, time out environment details, humidity control details, light control details, and label details.

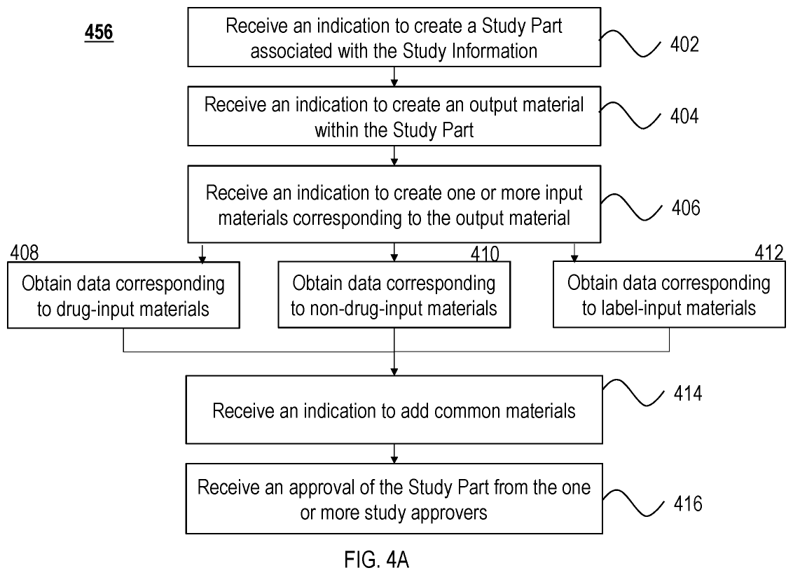

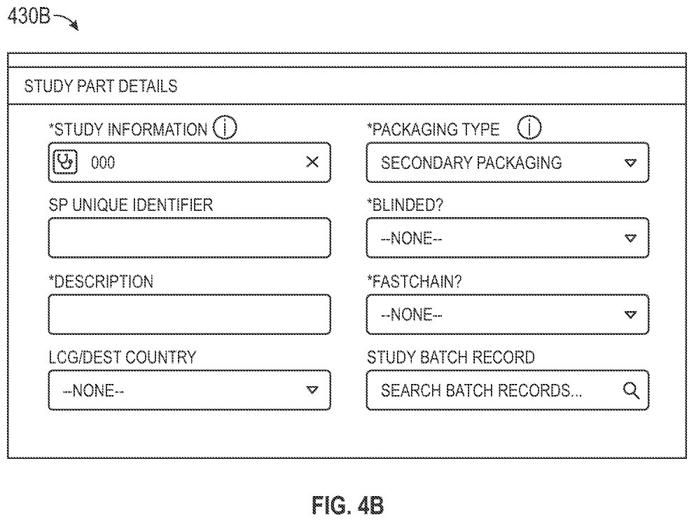

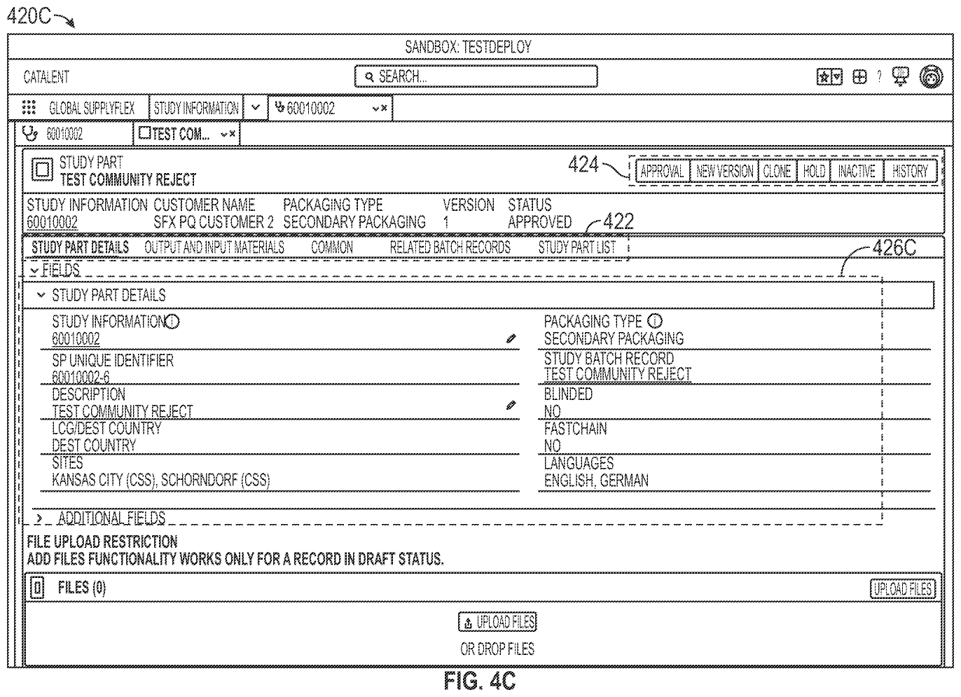

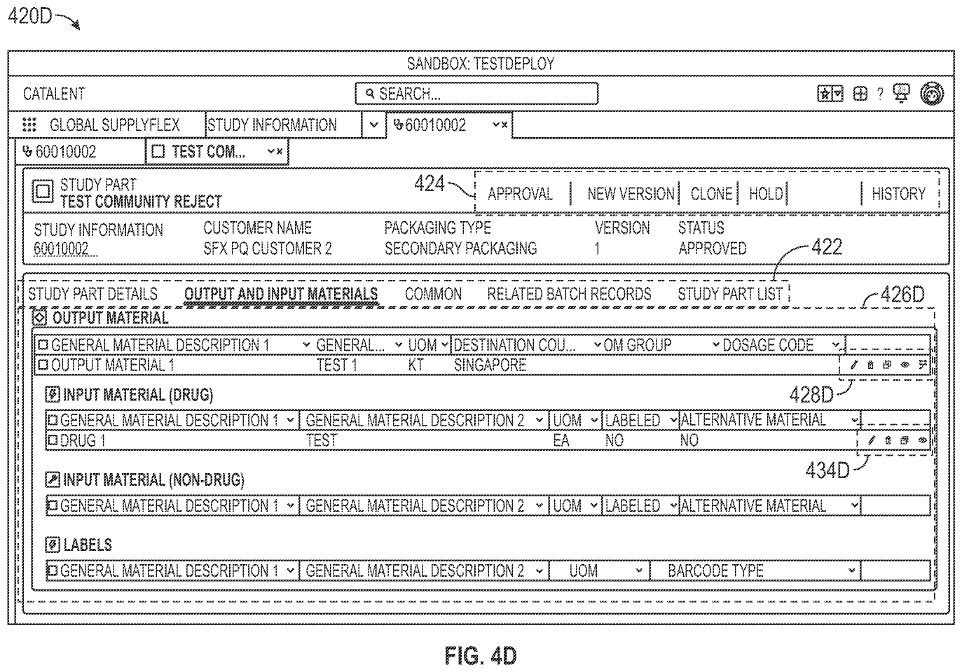

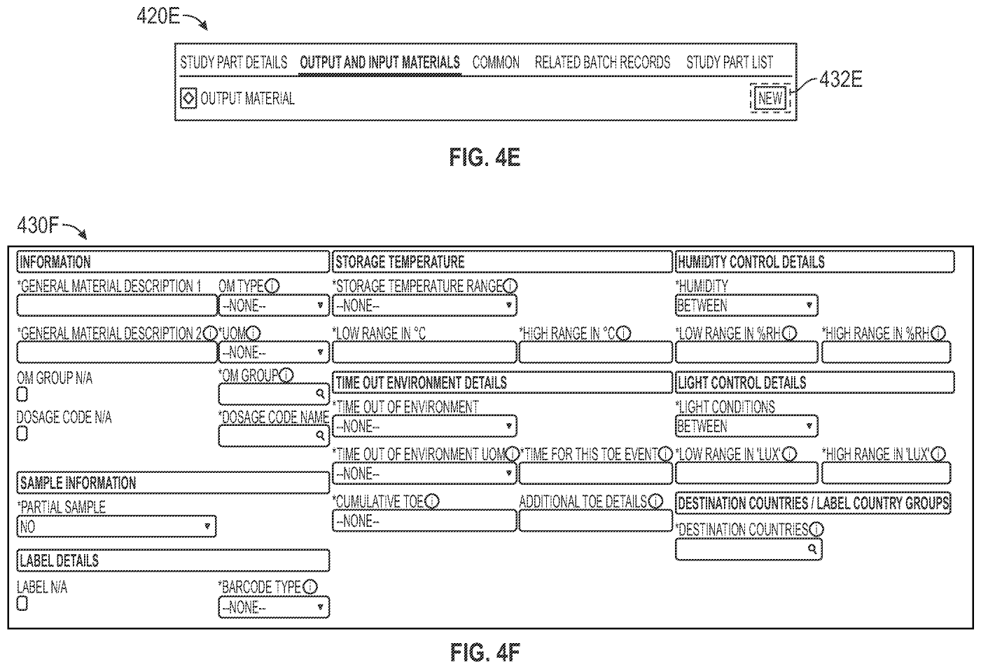

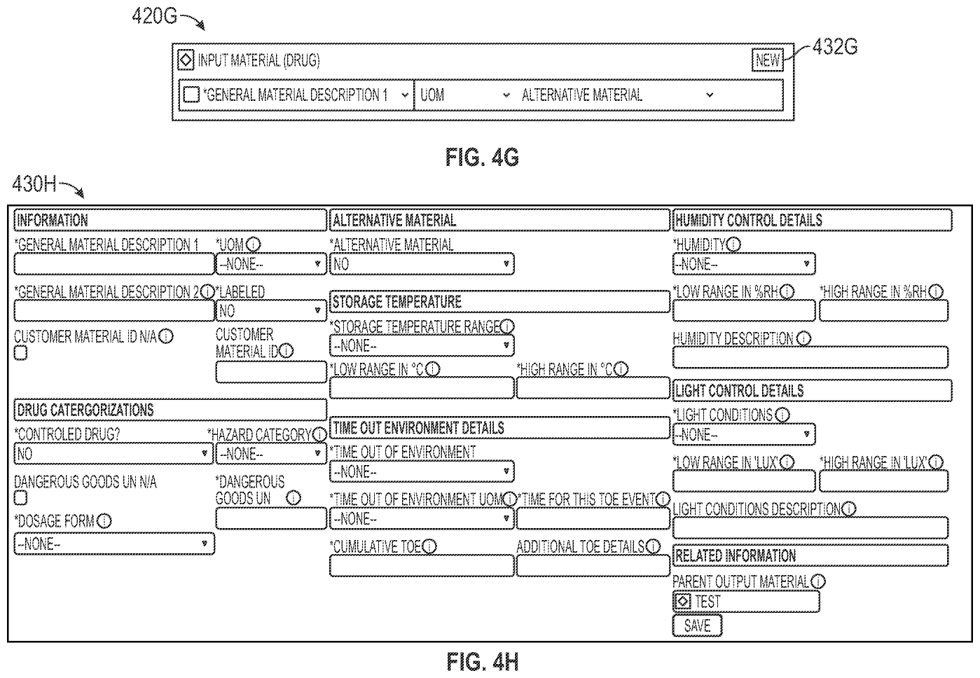

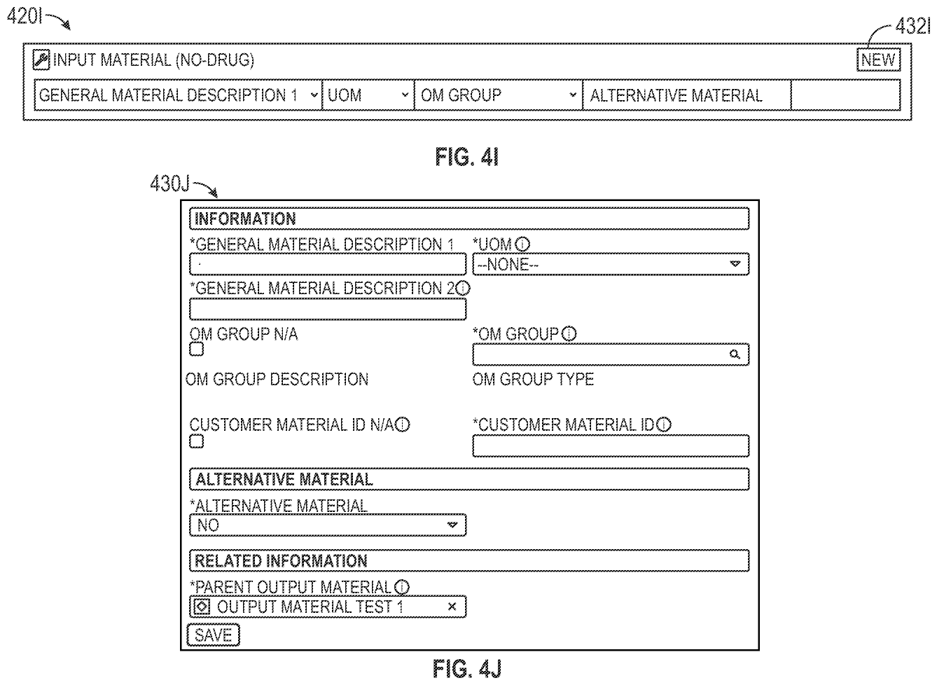

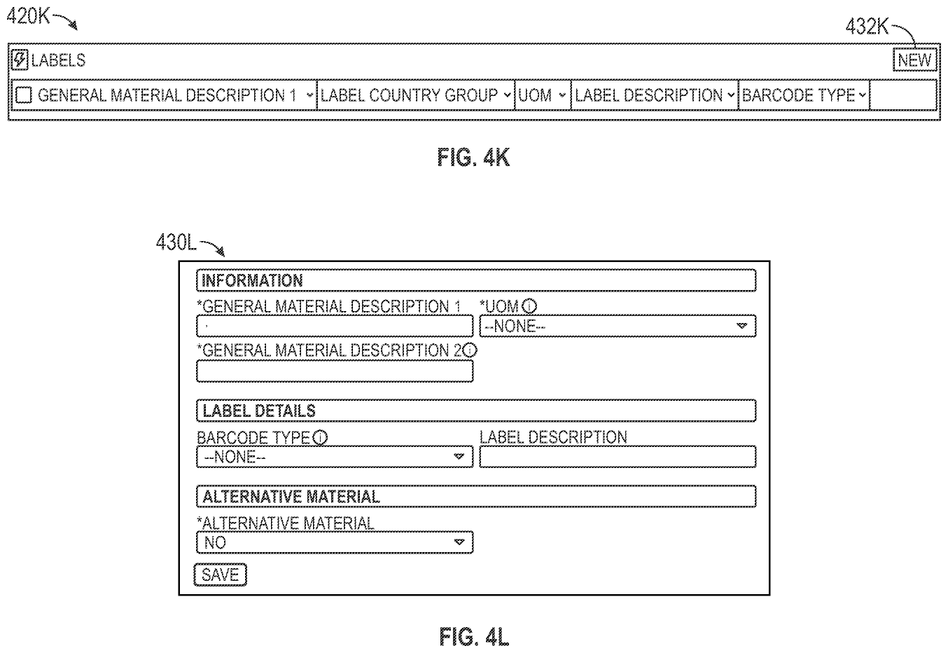

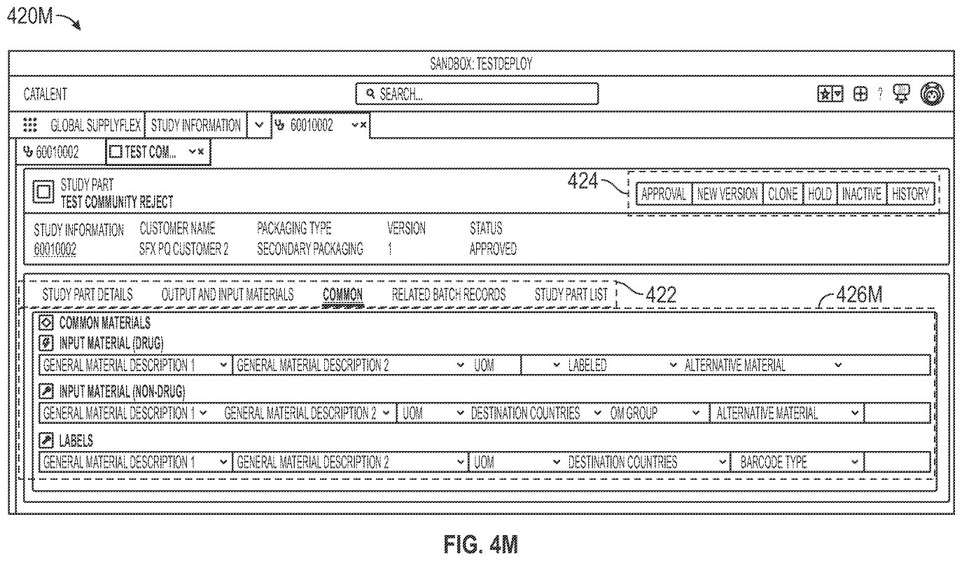

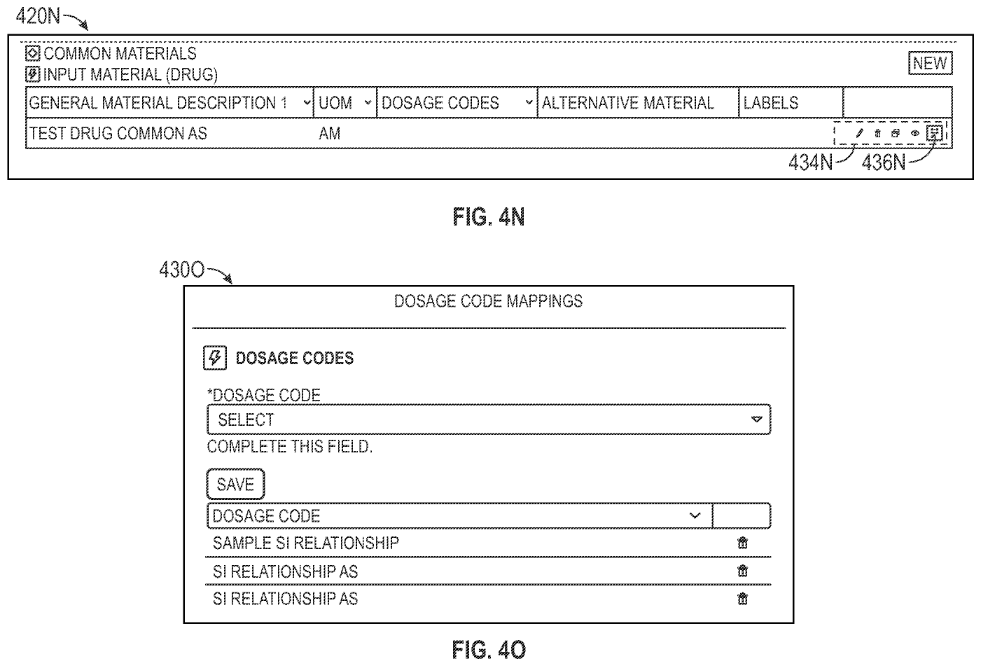

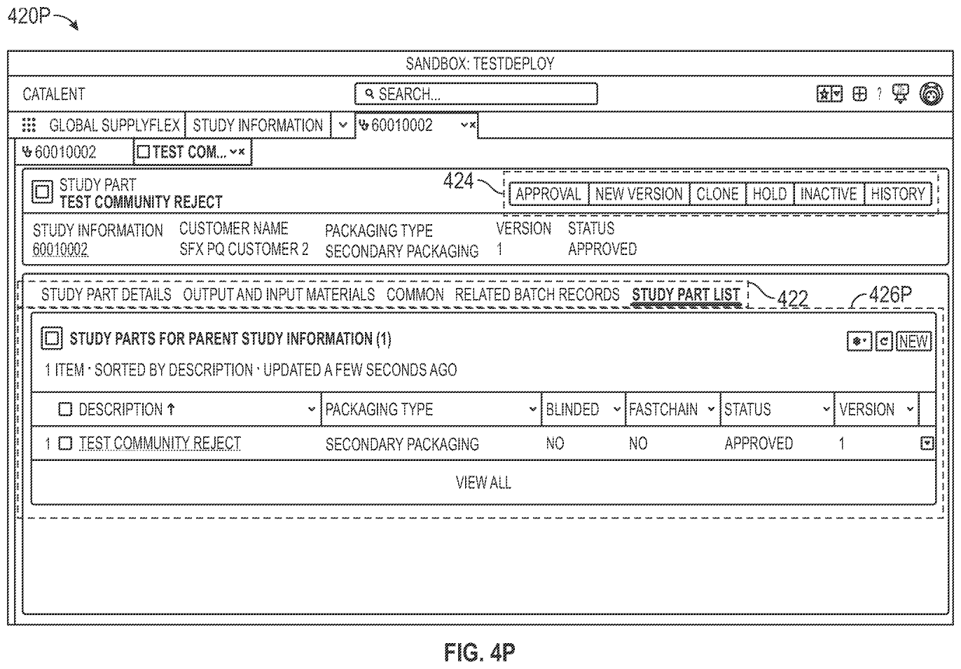

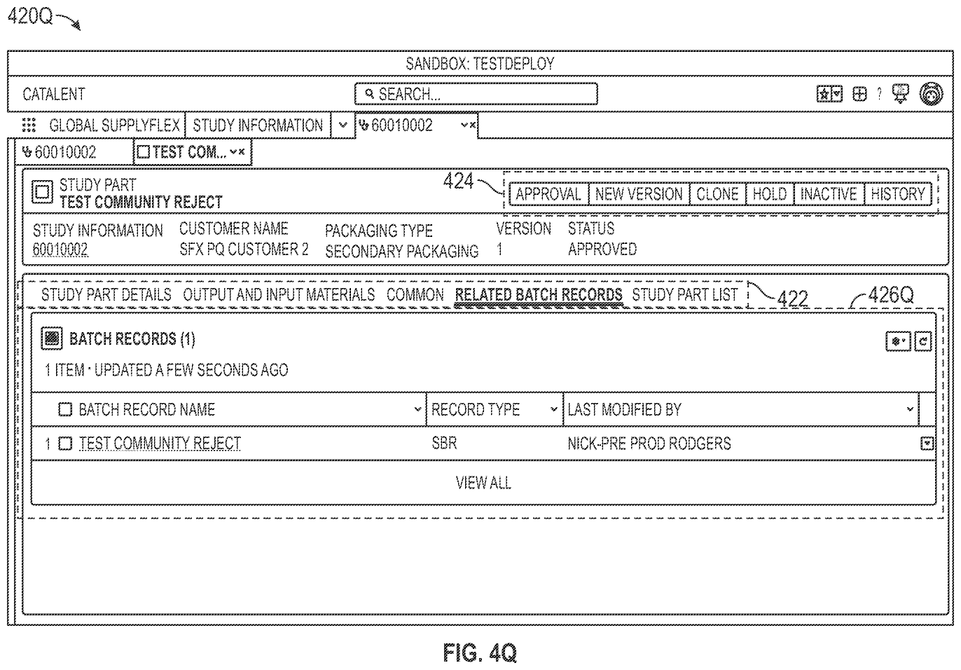

FIG. 4A illustrates process 456 for obtaining a study part 106, according to one or more embodiments of the present disclosure. In one or more embodiments, process 456 can correspond to Block 156 discussed above with respect to process 150 and process 160. Process 456 can be performed, for example, using one or more electronic devices implementing a software platform. In some examples, process 456 is performed using a client-server system, and the blocks of process 456 are divided up in any manner between the server and a client device. In other examples, the blocks of process 456 are divided up between the server and multiple client devices. In other examples, process 456 is performed using only a client device or only multiple client devices. In process 456, some blocks are, optionally, combined, the order of some blocks is, optionally, changed, and some blocks are, optionally, omitted. In some examples, additional steps may be performed in combination with the process 456. Accordingly, the operations as illustrated (and described in greater detail below) are exemplary by nature and, as such, should not be viewed as limiting.

At Block 402, the system can receive an indication to create a study part 106 associated with the study information 102. For example, while the system is displaying the study information UI (e.g., any one of pages 320A, 320F, 320G, 320H, 320J, 320K) the system can receive an indication that a production user has selected the Study Parts tab from the navigational user affordances 322. Upon receiving the selection of the Study Parts tab, the system can navigate to the Study Parts page 320L, as shown in FIG. 3L. The system may then receive an indication that the production user has clicked on the new-input user affordance 342L. In response to receiving a selection of the new-input user affordance 342L, the Production Facilitator UI can display a Study Parts Detail Window.

FIG. 4B illustrates a Study Parts Detail Window 430B, which includes a plurality of user affordances to receive an input from a production user. For example, the production user can enter details regarding the study part. As shown in the figure, the user affordances can include text entry boxes, drop down menus, and the like. In one or more examples, the study details can include but is not limited to a study information number, packaging type, a study part identifier, a description, a destination country/label country group, whether the study is blinded, whether the study is fastchain, and associate one or more study batch records. Upon receiving the study part details and receiving a user indication to save the same, the system can generate the study part.

FIG. 4C illustrates an exemplary Study Part Details page 420C associated with the Study Part UI in accordance with one or more examples of this disclosure. The Study Part Details page 420C can be presented via Production Facilitator UI 120. The Study Part Details page 420C can be configured to present details related to the study part. The Study Part Details page 420C can include a plurality of navigational user affordances 422, a plurality of operational user affordances 424, and a Study Part Detail information display region 426C. In one or more examples, the Study Part Detail information display region 426C can correspond to an area of the Study Part Details page 420C that displays the information regarding the study part. As shown in the figure, the Study Part Detail information display region 426C can display information included, but not limited to, a study information number, packaging type, a study part identifier, a description, one or more destination country/label country group, study part languages, one or more packaging sites, whether the study is blinded, whether the study is fastchain, and one or more study batch records.

In one or more examples, the Study Parts navigational user affordances 422 can include a plurality of user affordances, where each user affordance corresponds to a tab associated with the study information. As shown in the figure, the Study Parts navigational user affordances 422 can include a Study Part Detail tab, an Output and Input Materials tab, a Common Materials Tab, a Related Batch Records tab, and a Study List tab. The examples of navigational user affordances 322 are merely exemplary and more or less navigational user affordances can be used without departing from the scope of this disclosure.

In one or more examples, the Production Facilitator UI can receive an indication that a user has selected a Study Part navigation user affordance 422. Upon receiving the indication, the Study Parts UI can navigate to a page corresponding to the selected Study Part navigation user affordance 422. In one or more examples, the pages corresponding to the Study part navigational user affordances 422 can be configured to obtain information regarding a respective user affordance, e.g., Output and Input Materials, Common Materials, etc. For example, if the system receives an indication that a user has selected the Output and Input Materials tab, the system can update the Study Parts UI to display the page associated with the Output and Input Materials tab. The system can obtain information related to the Output and Input Materials via the page associated with the Output and Input Materials tab.

In one or more examples, the plurality of operational user affordances 424 can include a plurality of buttons that each correspond to a different action that can be implemented by a user. As shown in the figure, the operational user affordances 424 can include, but is not limited to an approval button, a new version button, a clone button, a hold button, an inactive button, a history button, and the like. In one or more embodiments, depending on the status of different items associated with study part 106, one or more of the operational user affordances 424 may be inactive and/or one or more of the operational user affordances may be removed or made visible. For example, the approval button may be inactive if the system has not received sufficient information related to the study part 106, e.g., information related to output materials, input materials, common materials, drug categorizations, alternative materials, storage temperature, time out environment details, humidity control details, light control details, and label details. The buttons associated with the operational user affordances 424 may have similar functions to those described above with respect to operational user affordances 324. In one or more examples, the clone button may be used to create a copy of a study part.

Referring back to FIG. 4A, at Block 404, the system can receive an indication to create an output material record within the Study Part. For example, the system can navigate to Output and Input Materials page, where the system can obtain information regarding the output and input materials from a production user. An output material record can correspond to a product that provided to a participant in a clinical trial study. For example, a 60-count bottle that is filled with 60 tablets of the drug (or placebo) provided to the participant in a clinical trial. In one or more examples, the system can receive an indication that the user selected the Output and Input Materials tab 420B from the navigational user affordances 422.

FIG. 4D illustrates an exemplary Output and Input Materials page 420D corresponding to the Output and Input Materials tab associated with the Study Parts UI. The Output and Input Materials page 420D can be presented via Production Facilitator UI 120 and be configured to obtain information related to the output and input materials associated with the study part. The Output and Input Materials page 420D can include a plurality of navigational user affordances 422, a plurality of operational user affordances 424, and an Output and Input Materials information display region 426D. In one or more examples, the Output and Input Materials information display region 426D can correspond to an area of the Study Part UI that displays the information regarding the input and output materials of the selected study part. As shown in the figure, the Output and Input Materials information display region 426D can display information included, but not limited to, one or more output material, a corresponding drug input material, a corresponding non-drug input material, and a corresponding label. The Output and Input Materials information display region 426D can further include a plurality of user affordances 428D, 434D.

As shown in the figure, the Output and Input Materials page 420D has been approved by a production user (e.g., approved status). However, while the Output and Input Materials page is in a draft status, the Production Facilitator UI may display a New Output Material user affordance. FIG. 4E illustrates an exemplary portion of an Output and Input Materials page 420E in a draft status. The Output and Input Materials page in a draft status can include a New Output Material user affordance 432E. In response to receiving an indication that the user has selected the New Output Material user affordance 432E, the system can display an Output Material Details Window.

FIG. 4F illustrates an exemplary Output Material Details Window 430F. As shown in the figure, the Output Material Details Window 430F can includes a plurality of user affordances to receive inputs from a production user regarding output material details to create an output material record. As shown in the figure, the user affordances can include text entry boxes, drop down menus, and the like. In one or more examples, the output material details can include, but is not limited to, one or more general material descriptions, an output material type, a UOM, a output material group, a dosage code, sample information, a destination country/label country group, a label, a barcode, a storage temperature range, a time out of environment information, humidity information, light information, and a customer material identifier.

In one or more examples, the system may not permit an output material record to be completed without receiving information associated with each of the fields included in the Output Material Details Window 430F. For example, the system can continue to present Output Material Details Window 430F until the production user has entered the corresponding details. In one or more examples, a multiple output materials can be created for a single study part. Upon creating an output material record, a user can navigate to the Output and Input Materials page to create a new output material record. In one or more examples, a user may not be permitted to create additional output material records associated with a study part once the study part is approved. In order to add a new output material record, a user may have to create a new version of the study part or clone the study part (e.g., using the corresponding operational user affordances 424).

Referring briefly to FIG. 4D, a user may be permitted to edit, delete, clone, preview, and view a relationship of an output material using a corresponding the Output Material user affordances 428D. For example, going from left to right, each user affordance 428D corresponds to a respective icon associated with an edit action, delete action, clone action, preview action, and relationship viewing action. In one or more examples, if a study part is approved, then the system may deactivate the edit, delete, and clone user affordances.

Upon receiving an indication that a user has selected the edit icon, the system can allow a user to modify information associated with the output material record. Upon receiving an indication that a user has selected the delete icon, the system can delete the corresponding output material record. If there are input material records associated with the deleted output material record, then those input material records can be removed from the Study Part. Upon receiving an indication that a user has selected the clone icon, the system can duplicate the output material record by creating a new output material record and updating the general material description field to differentiate the cloned output material record from the parent output material record. Upon receiving an indication that a user has selected the preview icon, the system can present the output material record in a read-only mode without the ability to update the record. This may be useful when the study part is approved, and a user wants to view the output material record. Upon receiving an indication that a user has selected the relationship viewing icon, the system can present the each of the output materials used within the same output material group.

At Block 406, the system can receive an indication to create one or more input material records for one or more input materials corresponding to the output material. In one or more examples, three types of input materials may be associated with a single output material. For example, referring to Output and Input Materials page 420D, each output material can be associated with a drug-input-material, non-drug-input-material, and label-input material.

At Block 408, the system can obtain data corresponding to drug input materials. FIG. 4G illustrates an exemplary portion of an Output and Input Materials page 420G that includes a New Input (Drug) Material user affordance 432G. The Input (Drug) Material can be correspond to a pharmaceutical drug and/or placebo. The New Input (Drug) Material user affordance can be associated with an output material that has been created at Block 404. The New Input (Drug) Material user affordance 432G can be used to create one or more input (drug) material records for an input (drug) material associated with an output material. In response to receiving an indication that the production user has selected the New Input (Drug) Material user affordance 432G, the system can display a Drug Input Material Details Window.

FIG. 4H illustrates an exemplary Input (Drug) Material Details Window 430H. As shown in the figure, the Input (Drug) Material Details Window 430H can include a plurality of input user affordances for a user to enter details to create the input (drug) material record. As shown in the figure, the user affordances can include text entry boxes, drop down menus, and the like. In one or more examples, the output material details can include but is not limited to one or more general material descriptions, label information, a UOM, a customer material identifier, drug categorization information, alternative material information, a storage temperature range, a time out of environment information, humidity information, light information, and the like.

In one or more examples, the system will not permit the input (drug) material record to be completed without receiving information associated with each of the fields included in the Input (Drug) Material Details Window 430H. In one or more examples, a single output material record can be associated with multiple input (drug) materials. In one or more examples, a user may not be permitted to create additional drug input material records once the study part is approved. In such examples, a user may create a new version of the study part or clone the study part (e.g., using the corresponding operational user affordances 424) to add a new input (drug) material record. As discussed above concerning the output materials, a user may be permitted to edit, delete, clone, and preview a drug input material record using a corresponding Input Material user affordances 434D.

At Block 410, the system can obtain data corresponding to non-drug input materials. FIG. 4I illustrates an exemplary portion of an Output and Input Materials page that includes a New Input (Non-Drug) Material user affordance 432I. The New Input (Non-Drug) Material user affordance 432I can be associated with an output material that has been created at Block 404. The New Input (Non-Drug) Material user affordance 432I can be used to create one or more input (non-drug) materials associated with an output material. Input (non-drug) materials can include one or more input materials that do not include pharmaceutical drugs, e.g., bottles, desiccants, closures, etc. In response to receiving an indication that the production user has selected the New Input (Non-Drug) Material user affordance 432I, the Production Facilitator UI can display an Input (Non-Drug) Material Details Window.

FIG. 4H illustrates an exemplary Input (Non-Drug) Material Details Window 430J. As shown in the figure, the Input (Non-Drug) Material Details Window 430J can include a plurality of input user affordances for a user to enter details regarding the input (non-drug) material record. As shown in the figure, the user affordances can include text entry boxes, drop down menus, and the like. In one or more examples, the output material details can include but is not limited to one or more general material descriptions, output material group information, a UOM, a customer material identifier, alternative material information, destination countries/label country groups, and the like.

In one or more examples, the system will not permit the input (non-drug) material record to be completed without receiving information associated with each of the fields included in the Non-Drug Input Material Details Window 430J. In one or more examples, multiple the input (non-drug) materials can be associated with a single output material record. Upon creating a the input (non-drug) material record, a production user can navigate to the Output and Input Materials page, e.g., 420I, to create a new the input (non-drug) material record, as described above. In one or more examples, a user may not be permitted to create additional the input (non-drug) material records once the study part is approved. In such examples, a user may to create a new version of the study part or clone the study part (e.g., using the corresponding operational user affordances 424) to add a new the input (non-drug) material record. As discussed above concerning the output materials, a user may be permitted to edit, delete, clone, and preview a non-drug input material record using a corresponding Input Material user affordances 434D.

At Block 412, the system can obtain data corresponding to input (label) materials. FIG. 4K illustrates an exemplary portion of an Output and Input Materials page 420K that includes a New Input (Label) Material user affordance 432K. The New Input (Label) Material user affordance 432K can be associated with an output material that has been created at Block 404. In one or more examples, a label is available to be created for secondary packaging, e.g., bottles, cardboard boxes, plastic boxes, paperboard boxes, and the like. The New Input (Label) Material user affordance 432K can be used to create one or more label input material records associated with an output material. In response to receiving an indication that the production user has selected the New Input (Label) Material user affordance 432K, the system can display an Input (Label) Material Details Window.

FIG. 4L illustrates an exemplary Input (Label) Material Details Window 430L. As shown in the figure, the Input (Label) Material Details Window 430L can includes a plurality of input user affordances for a user to enter details regarding the input (label) material record. As shown in the figure, the user affordances can include text entry boxes, drop down menus, and the like. In one or more examples, the output material details obtained via the Input (Label) Material Details Window 430L can include but is not limited to one or more general material descriptions, a UOM, barcode information, and the like.

In one or more examples, the system may not permit the label input material record to be completed without receiving information associated with each of the fields included in the Label Input Material Details Window 430L. In one or more examples, multiple input (label) material records can be associated with a single output material record. Upon creating an input (label) material record, a user can navigate to the Output and Input Materials page to create a new input (label) material record, as described above. In one or more examples, a user may not be permitted to create additional input (label) material records once the study part is approved. In such examples, a user may create a new version of the study part or clone the study part (e.g., using the corresponding operational user affordances 424) to add a new input (label) material record. As discussed above, a user may be permitted to edit, delete, clone, and preview a non-drug input material record using a corresponding Input Material user affordances 434D.

At Block 414, the system can receive an indication to add common materials to a study part. In one or more examples, the same common material record can be associated with multiple output material records. For example, a study part includes two output materials, each output material including a respective 60-count bottle. In such example, rather than create the same input material record multiple times under different output material records, the user can create a single input material record in the Common section. In one or more examples, the common material records can be created as described above with respect to the input material records (e.g., Blocks 406-412). Mapping the common material records to corresponding output materials can occur when the batch record is created. Embodiments according to the present disclosure can include three types of common material records created for the Study Part: common (drug) material, common (non-drug) material, and common (label) input material. The common (label) input materials may be associated with secondary packaging.

The system can receive an indication to add common materials to a study part. For example, the system can receive an indication that the production user selected the Common Materials tab from the navigational user affordances 422. FIG. 4M illustrates an exemplary Common Materials page 420M corresponding to the Common Materials tab associated with the Study Parts UI. The Common Materials page 420M can be presented via Production Facilitator UI 120 and be configured to obtain information related to the common materials associated with the study part. Common materials may correspond to a material that is used in more than one output material. The Common Materials page 420M can include a plurality of Study Part navigational user affordances 422, a plurality of operational user affordances 424, and a Common Material information display region 426M. In one or more examples, the Common Material information display region 426M can correspond to an area of the Study Part UI that displays the information regarding the common materials. In one or more examples, the Common Materials page 420M can include information similar to the input material information (e.g., input (drug) materials, input (non-drug) materials, and input (label) materials) displayed by the Input and Output Materials page 420D, (e.g., corresponding to the common (drug) materials, common (non-drug) materials, and common (label) materials).

One difference between the Common Materials page 420M and the Output and Input Materials page 420D is that the Common Materials include one or more user affordances for linking the common material to corresponding output materials. As shown in the figure, the Common Materials page 420M has been approved by a production user (e.g., approved status). However, while the Common Materials page 420M is in a draft status, the Common Materials page can be used to link the common input (drug) materials to a dosage code; the common input (non-drug) materials to an output material group or destination/label country group; and/or the common input (label) materials to a destination/label country group.

For example, while the Common Materials page is in a draft status, the system may display a common material mapping user affordance. FIG. 4N illustrates an exemplary portion of a Common Materials page 420N corresponding to a common (drug) material from a Common Materials page in a draft status. As shown in the figure, the Common Materials page can include Common (Drug) Materials user affordances 434N, similar to the Input Material user affordances 434D. However, one distinction is the inclusion of a mapping user affordance 436N in the Common (Drug) Materials user affordances 434N. The mapping user affordance 436N can be used to map a respective Common Material (e.g., common drug input material) to one or more output materials.

FIG. 4O illustrates an exemplary Drug-Common Mapping Window 430O. The system can receive a user input via the Drug-Common Mapping Window 430O to map the common (drug) material to one or more dosage codes associated with the study part.

Regarding the common (non-drug) materials, the Common Materials page (e.g., while in draft status) can be configured to receive an indication of an output material group or a destination country/label country group. For example, a production user can input an output material group or a destination country/label country group for a common (non-drug) material via one or more user affordances. Regarding the common (label) materials, the Common Materials page (e.g., while in draft status) can be configured to receive an indication of a destination country/label country group. For example, a production user can input a destination country/label country group for a common (label) material via one or more user affordances. While the specific UIs for mapping the common (non-drug) materials and common (label) materials are not shown, a skilled artisan will understand that the UIs may be similar to those described with respect to FIGS. 4N and 4O.

At Block 416, the system can receive an approval of the Study Part from the one or more study approvers. In one or more examples, the system will not permit an approval to be accessed without receiving completed output and input materials information. The approval process may be similar to that described above with respect to Block 312 of process 354. For example, the system can receive an indication that a user has selected the Approval button from the operational user affordances 424. The Approval button can be selected from any of the pages and/or tabs associated with the Study Part UI. Upon receiving the selection of the Approval button, the system can present an approval report. The approval report can include the study part information associated with each of the Information Display regions across the various Study Part tabs. For example, the approval report can include the information included in one or more of the Study Part Details information display region 426C, the Output and Input Materials information display region 426D, the Common Materials information display region 326M, the Study part List information display region 426P, and the Related Batch Records information display region 426Q.

Once a study part is approved, then the system may not permit changes to be made to the approved version of the study information. If a production user desires to make changes to a study part 104, then the production user can create a new version of the study part or clone the study part. For example, the system can create a new version of the study part by receiving a selection by the user of the New Version button from the operational user affordances 424 associated with the Study Part UI. Similarly, the system can clone an existing study part by receiving a selection by the user of the Clone button from the operational user affordances 424 associated with the Study Part UI. Changes to the new version of the study information can be made in accordance with the description provided above, e.g., with respect to process 456. Once a new version is created, the previous version will remain the controlling version of the study information until the new version is approved, as described above with respect to Block 416.

In one or more examples, the system can present a study part list to a production user. FIG. 4P illustrates an exemplary Study Part List page 420P, according to embodiments of this disclosure. Study Part List page 420P includes an approved study part. The Study Part List page 420P can be presented via Production Facilitator UI 120 and configured to display the study parts that have been created. As shown in the figure, the Study Part List page 420P can include the plurality of navigational user affordances 422, operational user affordances 424, and a Study Part List information display region 426P. The plurality of navigational user affordances 422 and operational user affordances 424 may be similar to those described above. The Study Part List information display region 426P can display the study parts associated with the same study information. For example, the Study Part List information can include for each study part, but is not limited to, a description, a packaging type, a blinding status, a fastchain status, an approval status, and a version.

In one or more examples, the system can present related batch records to a user. FIG. 4Q illustrates an exemplary Related Batch Record page 420Q, according to embodiments of this disclosure. The Related Batch Record page 420Q can be presented via Production Facilitator UI 120 and configured to display the batch records associated with the study part. As shown in the figure, the Related Batch Record page 420Q can include the plurality of navigational user affordances 422, operational user affordances 424, and a Related Batch Record information display region 426Q. The plurality of navigational user affordances 422 and operational user affordances 424 may be similar to those described above. The Related Batch Record information display region 426Q can display the batch records associated with the study part. In one or more examples, the system can navigate to a batch record via the Related Batch Record page 420Q (e.g., by receiving a user selection of a link associated with the listed batch record).

### Study Batch Record

A study batch record 108 can include one or more study instructions 110 and a bill of materials 112. In one or more examples, each study part 106 can be associated with a respective study batch record 108. In one or more examples, the system can generate a study batch record 108 based on a completed study part 106. In one or more examples, the system can automatically generate the study batch record 108 upon receiving an approval of the completed study part 106, e.g., from a customer approver. In one or more examples, the study batch record 108 can acts as a container for the study instructions 110 and the bill of materials 112. In one or more examples, the study bath record can be configured to link and/or display the most recent version of the study instructions 110 with the bill of materials 112.

FIGS. 5A-1 and 5A-2 illustrate an exemplary Batch Record Detail page 520A of a Study Batch Record UI, according to one or more embodiments of the present disclosure. The Batch Record Detail page 520A can be presented via Production Facilitator UI 120 and configured to present batch record information related to an approved study part. As shown in the figure, the Batch Record Detail page 520A can include a plurality of navigational user affordances 522, a plurality of operational user affordances 524, and a Batch Record Detail information display region 526A.

In one or more examples, the information display region 526A can correspond to an area of the Batch Record Detail page that displays information related to the study batch record. As shown in the figure, the Study Batch Record information display region 526A can display information included, but not limited to, instructions, room processing temperature, room processing humidity, room processing light conditions, the batch record name, a blinding status, a study batch record identifier, a project number, the corresponding study part, and a customer.

In one or more examples, the navigational user affordances 522 can include a plurality of user affordances, where each user affordance corresponds to a tab and respective page associated with the study information. As shown in the figure, the navigational user affordances 522 can include a Study Batch Record tab, an Instructions and Equipment Group tab, a Bill of Materials tab, a Related Study Parts tab, and a Related Batch Record tab. The examples of navigational user affordances 522 are merely exemplary and more or less navigational user affordances can be used without departing from the scope of this disclosure. The tabs can be used to navigate between the respective pages of the Study Batch UI.

In one or more examples, the plurality of operational user affordances 524 can include a plurality of buttons that each correspond to a different action that can be implemented by a production user. As shown in the figure, the operational user affordances 524 can include, but are not limited to an approval button, a new version button, a create lot assignment button, a hold button, an inactive button, a history button, and the like. In one or more embodiments, depending on the status of different items associated with the study batch record 108, one or more of the operational user affordances 524 may be inactive and/or one or more of the operational user affordances may be removed or made visible. The operational user affordances 524 can correspond to the functions of the operational user affordances described above.

In one or more examples, the process of generating a study batch record can correspond to Block 158 discussed above with respect to process 150 and process 160. In one or more examples, the system can obtain information associated with the study batch record via the Study Batch Record UI.

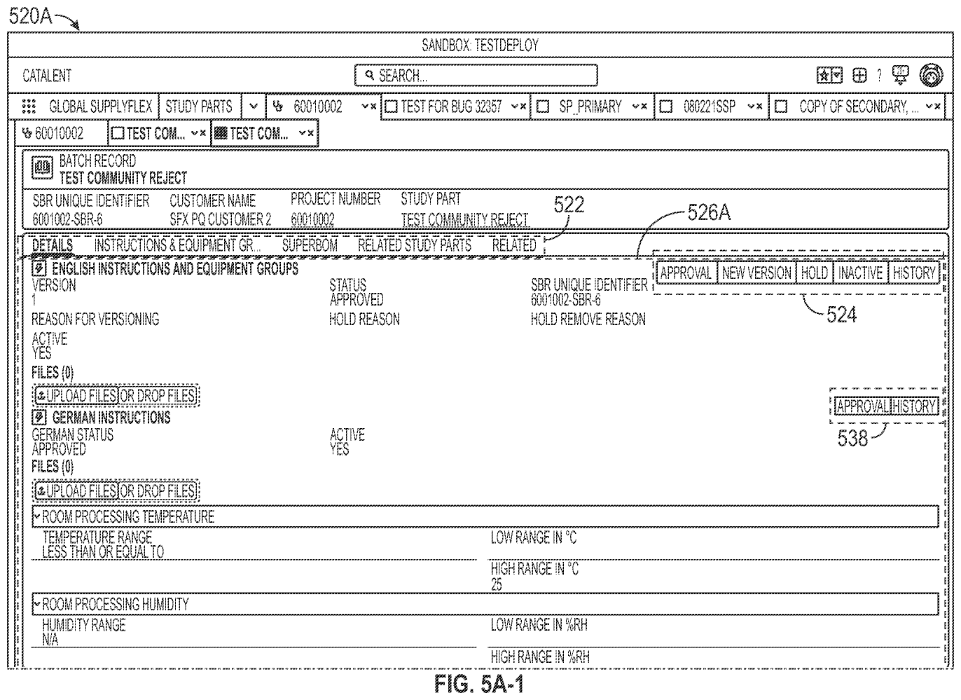

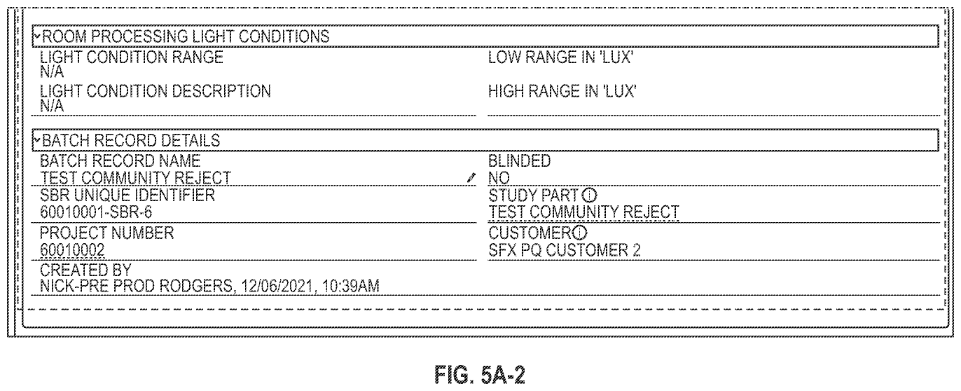

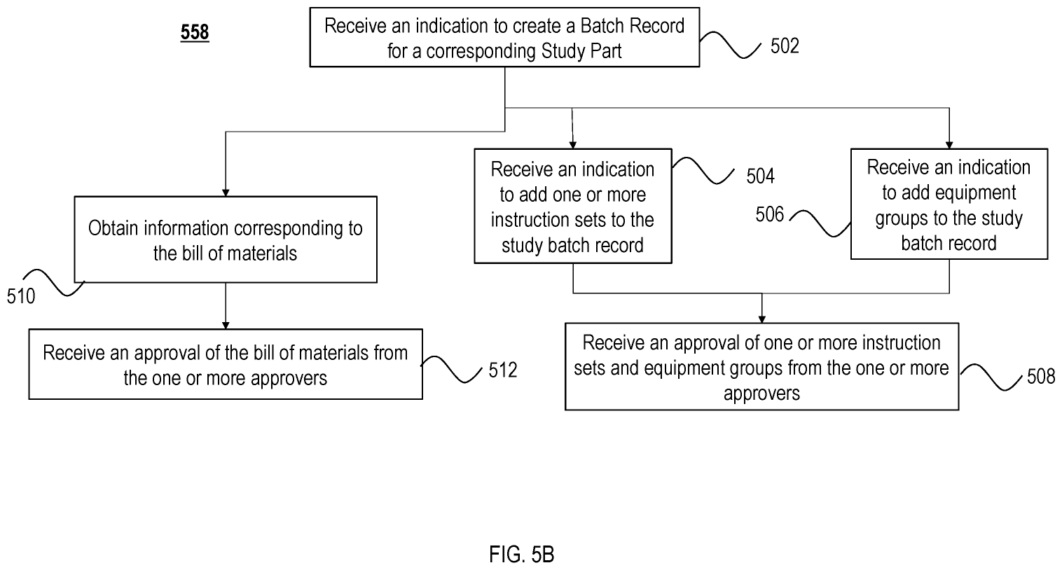

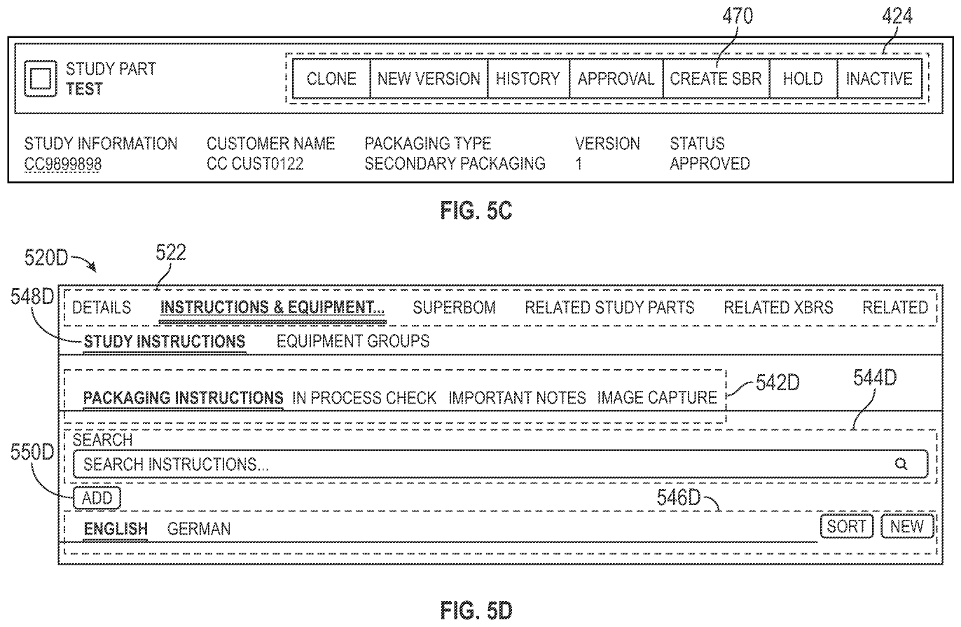

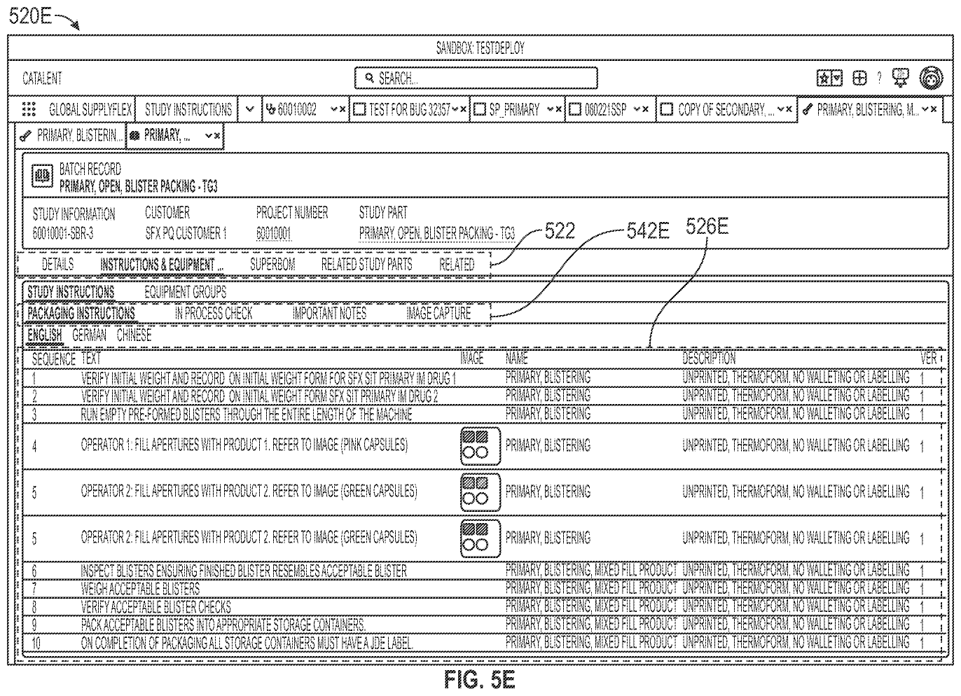

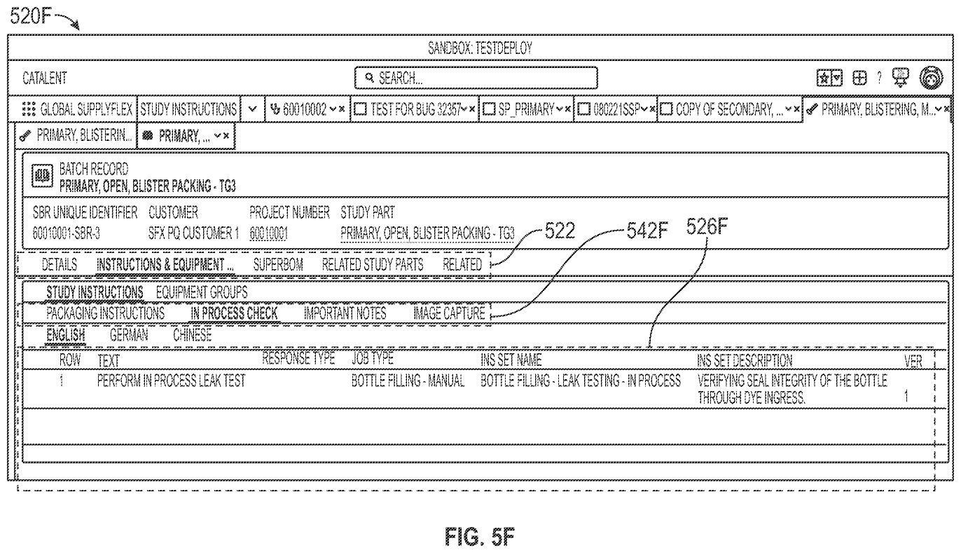

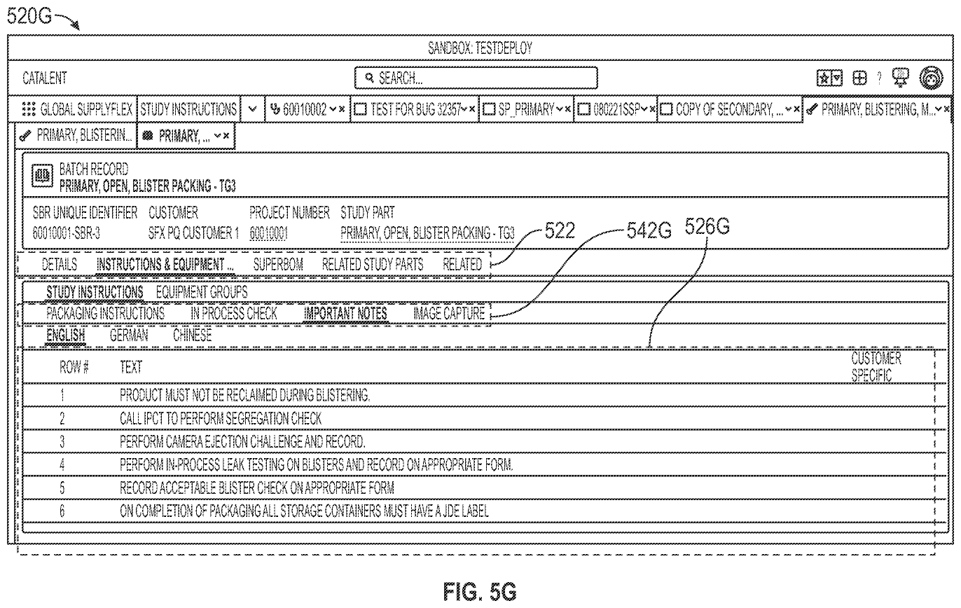

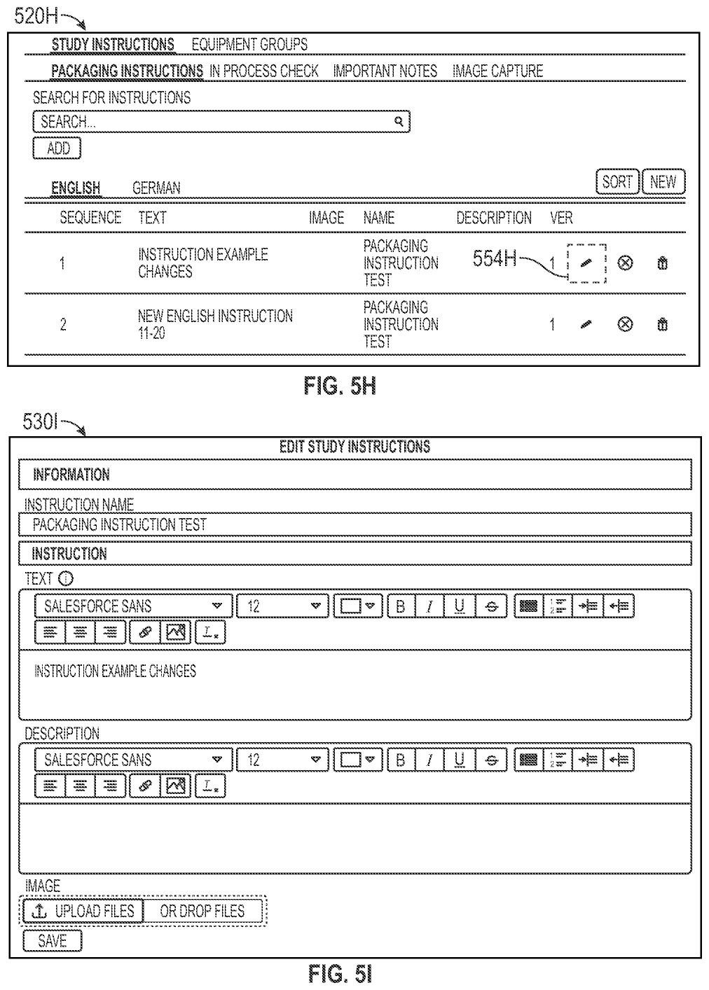

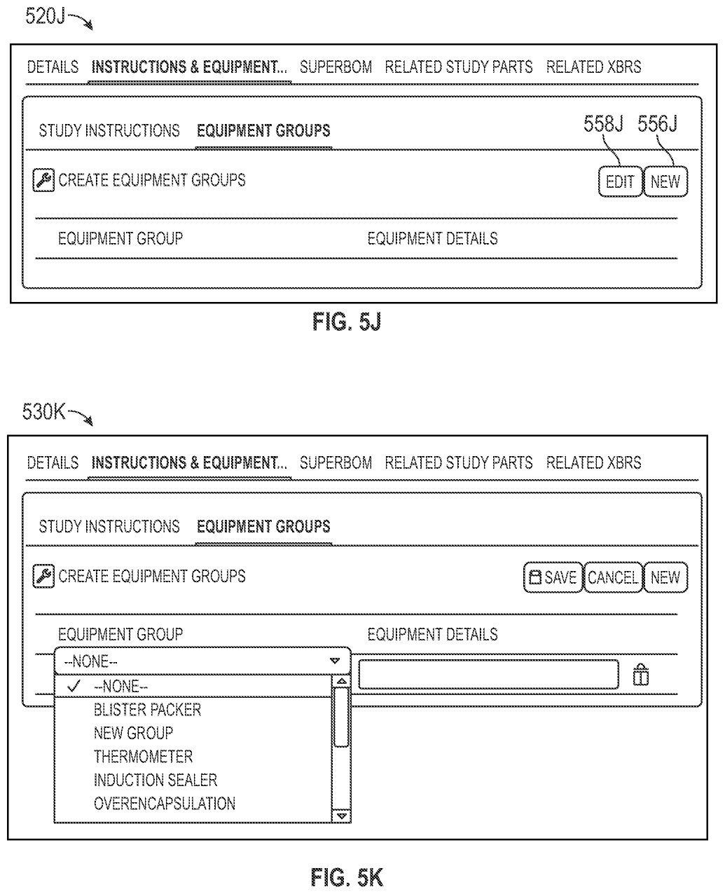

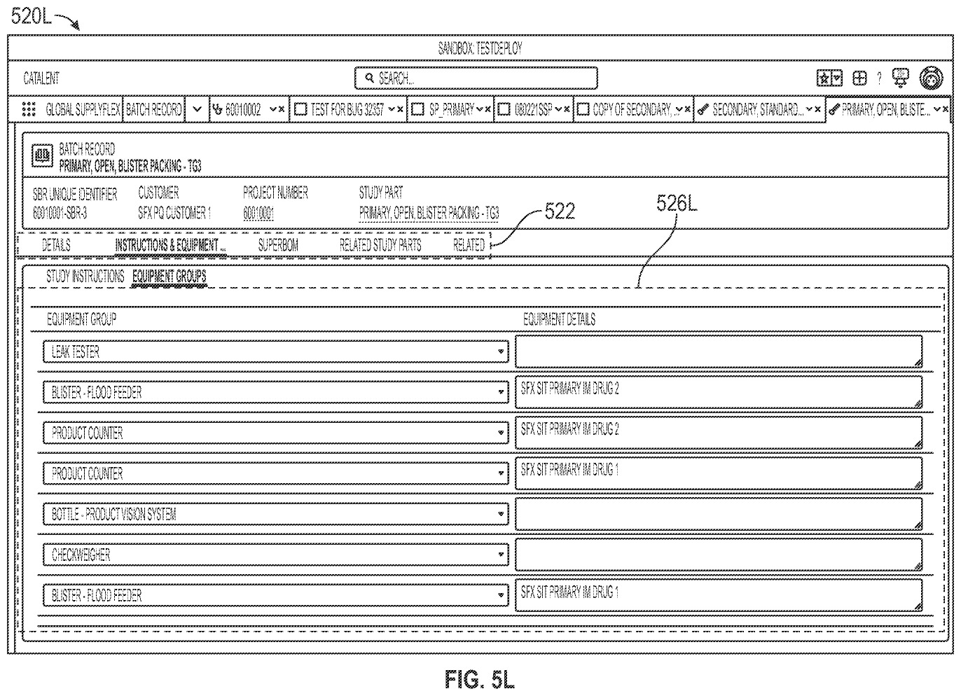

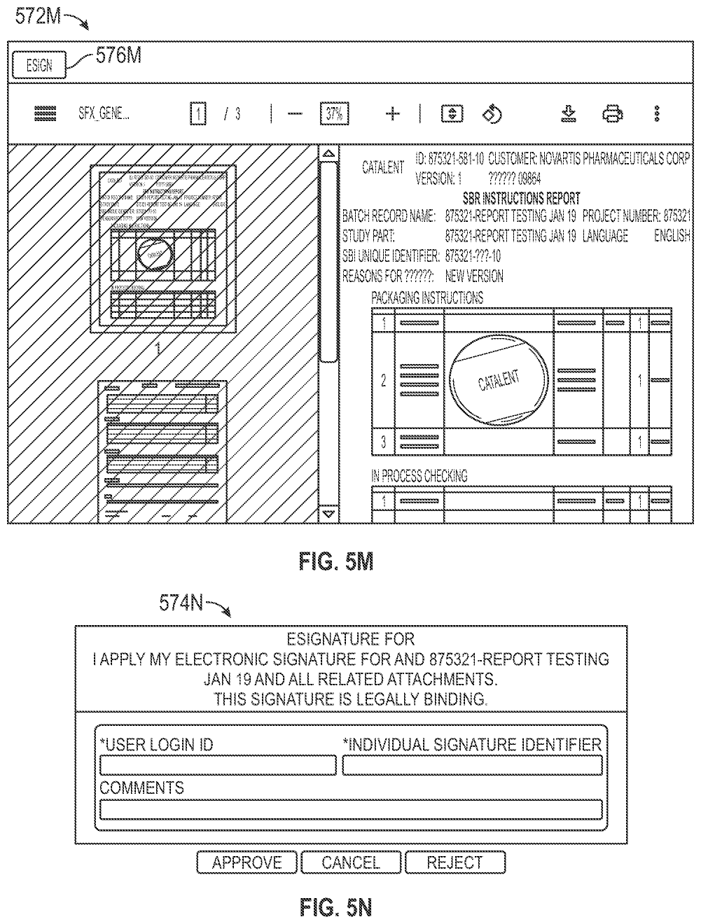

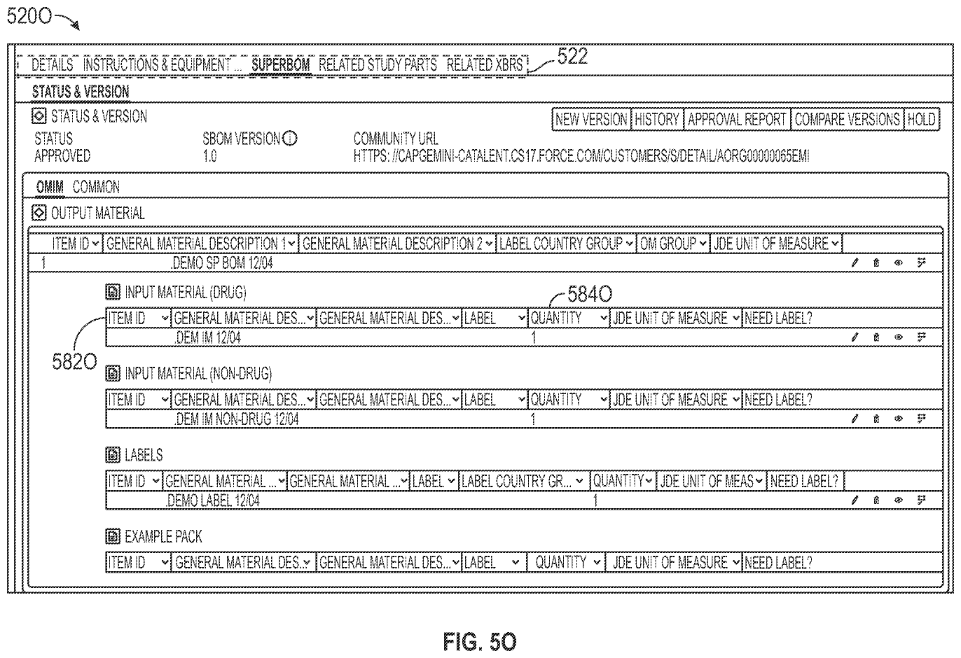

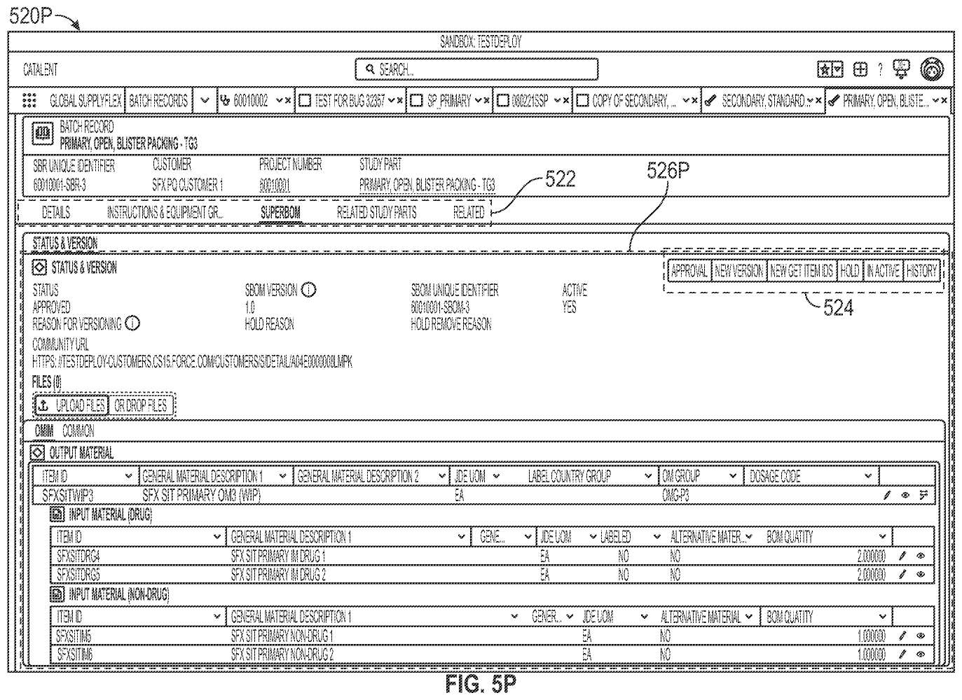

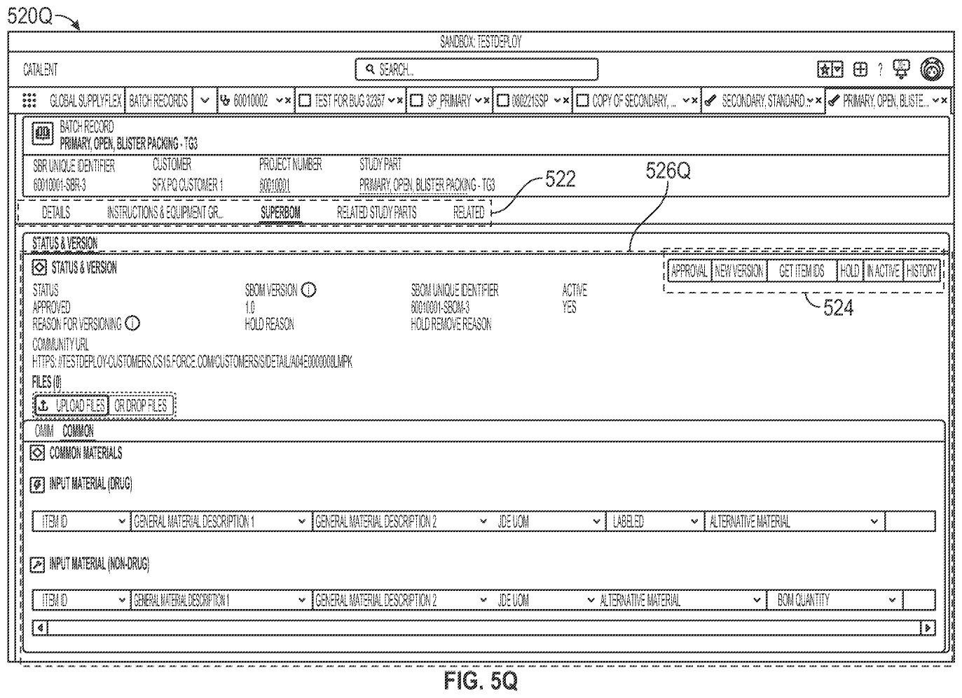

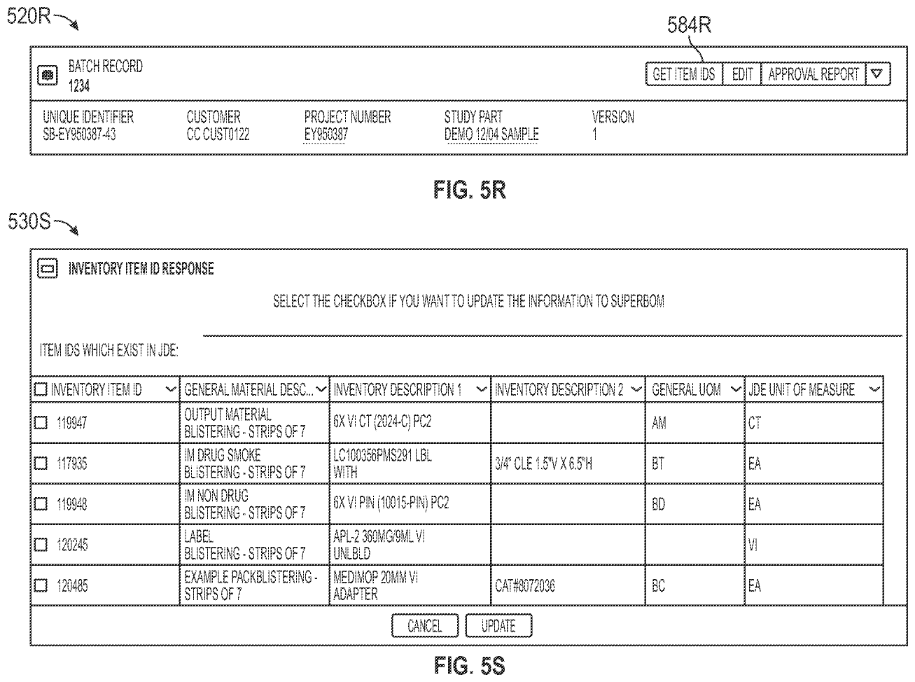

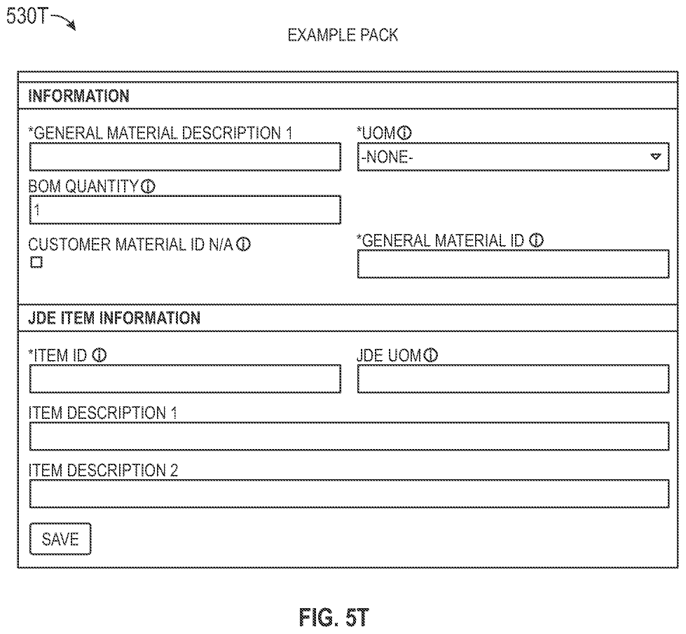

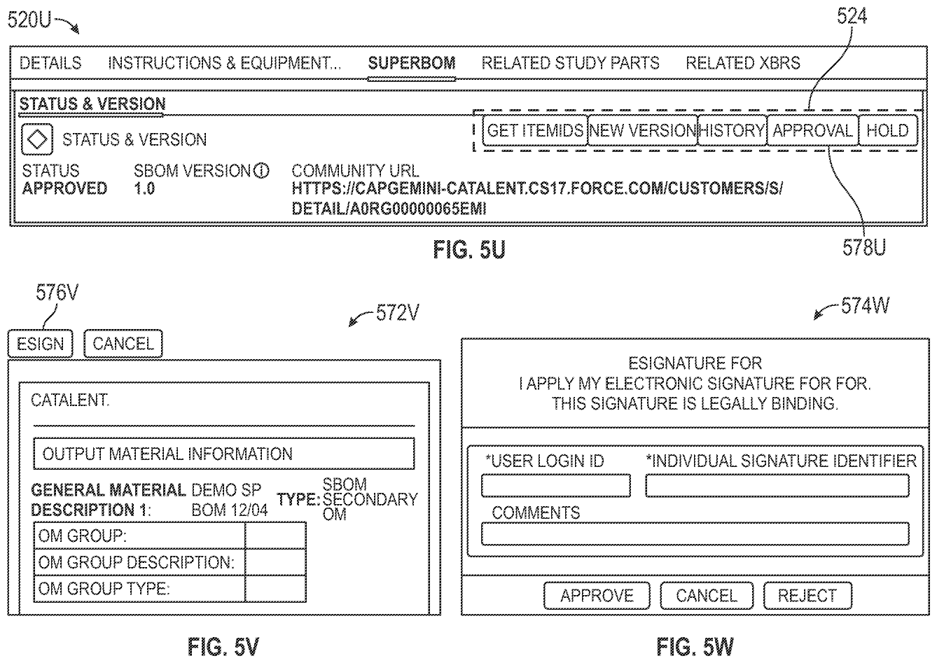

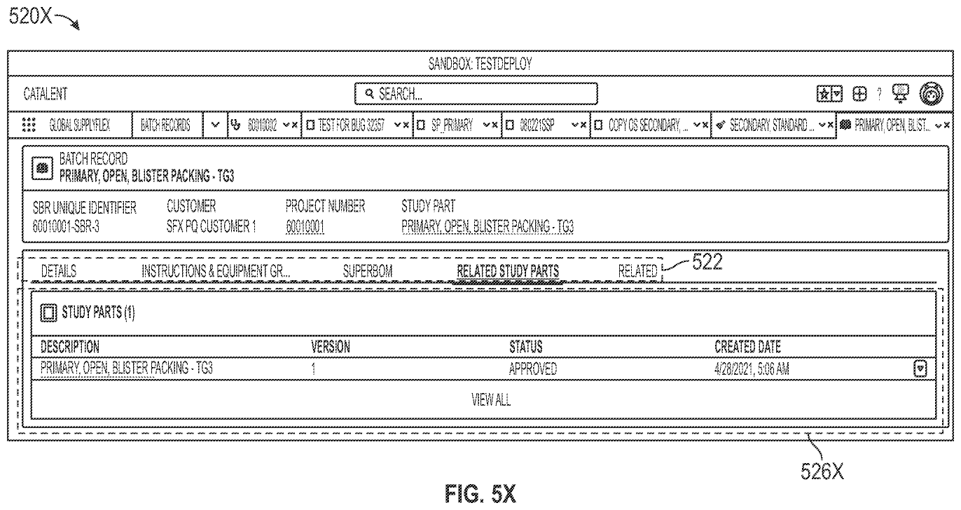

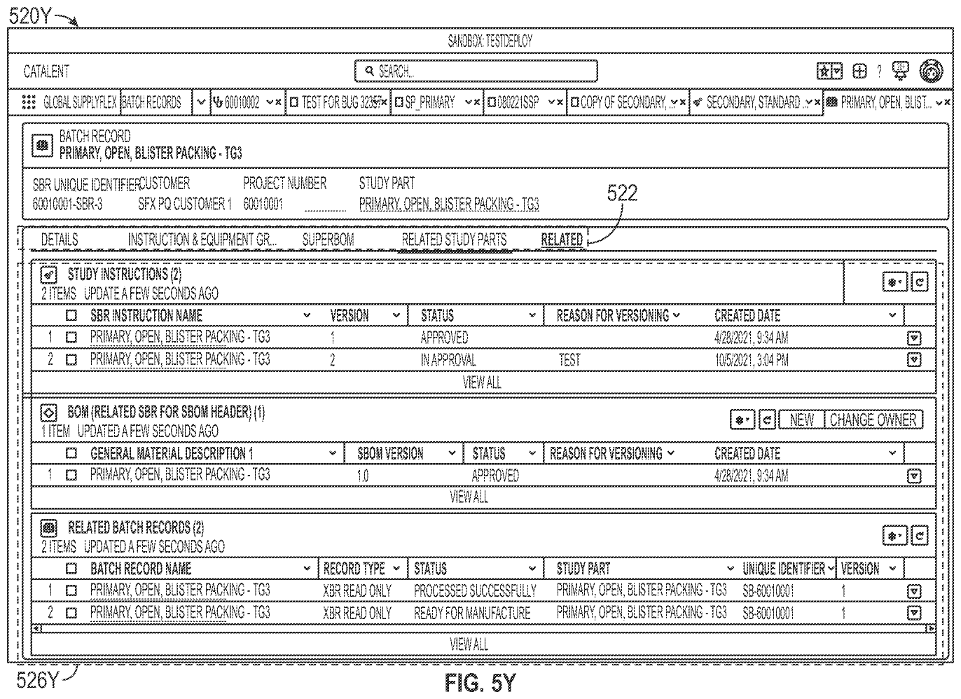

FIG. 5B illustrates process 558 for generating a study batch record, according to one or more embodiments of the present disclosure. In one or more embodiments, process 558 can correspond to Block 158 discussed above with respect to process 150. Process 558 can be performed, for example, using one or more electronic devices implementing a software platform. In some examples, process 558 is performed using a client-server system, and the blocks of process 558 are divided up in any manner between the server and a client device. In other examples, the blocks of process 558 are divided up between the server and multiple client devices. In other examples, process 558 is performed using only a client device or only multiple client devices. In process 558, some blocks are, optionally, combined, the order of some blocks is, optionally, changed, and some blocks are, optionally, omitted. In some examples, additional steps may be performed in combination with the process 558. Accordingly, the operations as illustrated (and described in greater detail below) are exemplary by nature and, as such, should not be viewed as limiting.

At Block 502, the system can receive an indication to create a study batch record according to one or more embodiments of this disclosure. In one or more examples, the study batch record 108 can be created from an approved study part 106. In one or more examples, the system can create a version of the study instructions 110 and bill of materials 112 when the study batch record 108 is created. In one or more examples, upon receiving approval of a study part 106, a new user affordance can appear among the Study Part operational user affordances. FIG. 5C illustrates an exemplary operational user affordances 424 of the Study Part UI, as it may appear after approval of a study part. As shown in the figure, the ‘Create SBR’ user affordance 470 is visible. In one or more examples, when the corresponding study part is versioned up, subsequent versions may be associated with the same study batch record. Upon receiving a selection of the Create SBR user affordance 470, the system can generate a Study Batch Record. In one or more examples, information associated with the corresponding study part may import to one or more fields associated with the study batch record 108.

At Block 504, the system can receive an indication to add one or more instruction sets to the study batch record. For example, the system can receive an indication that a user has selected the Instructions and Equipment Group tab from the navigational user affordances 522. Upon receiving the selection of the Instructions and Equipment Group tab, the system can navigate to the Instructions and Equipment Group (Study Instructions) page 520D, as shown in FIG. 5D. Instructions and Equipment Group (Study Instructions) page 520D corresponds to a draft Study Batch Record UI (e.g., not an approved study batch record).

As shown in the figure, the Instructions and Equipment Group (Study Instructions) page 520D can include the plurality of navigational user affordances 522, an Instruction-Type navigational user affordances 542D, a search user affordance 544D, and one or more language-selection user affordances 546D. The second plurality of navigation affordances 542D can each correspond to one of the four categories of study instructions: packaging instructions, in-process checklist, important notes, and image capture.

In one or more examples, the system can receive packaging instructions to be associated with the study batch record. For example, the packaging instructions can be added by receiving a searching for instructions via the search user affordance 544D or adding an instruction directly via the add instruction user affordance 550D. Once the instructions are added, the instructions may appear beneath the language-selection user affordances 546D. In one or more examples, the packaging instructions can be generated in English before creating translations in other languages. In one or more examples the packaging instructions can be imported from an instruction repository, e.g., packaging instructions 128 and/or instruction sets 130 associated with master data 102. In one or more examples, the system can obtain the packaging instructions directly from the production user. In one or more examples, the in-process checklist information can be completed based on the selected packaging instructions. In one or more examples, the important notes can be imported from the selected packaging instructions, packaging instructions 128 and/or instruction sets 130 associated with master data 102. In one or more examples, the important notes may be specific to a particular customer. In one or more examples, the image capture information can be completed based on the selected packaging instructions.

FIG. 5E illustrates an exemplary Instructions and Equipment (Study Instructions-Packaging Instructions) page 520E from the Study Batch Record UI. Instructions and Equipment (Study Instructions-Packaging Instructions) page 520E may correspond to an approved study Instructions and Equipment. As shown in the figure, the Instructions and Equipment (Study Instructions-Packaging Instructions) page 520E can include the plurality of navigational user affordances 522, the Instruction-Type navigational user affordances 542E, and Instructions and Equipment (Study Instructions-Packaging Instructions) display information 526E. As shown in the figure, the Instructions and Equipment (Study Instructions-Packaging Instructions) display information 526E can include, but is not limited to, the sequence, text, images, name, description, and version for each step of the packaging instructions.

In one or more examples, the packaging instructions can be provided in multiple languages. In some examples, the translations may depend on the packaging sites associated with the study batch record 108 and/or study part 106. In such examples, the text associated with the study instructions may be translated based on the English packaging instructions to the selected language. For example, a German translation German translation can be created based on the English packaging instructions.

FIG. 5F illustrates an exemplary Instructions and Equipment (Study Instructions—In Process Check) page 520F from the Study Batch Record UI. Instructions and Equipment (Study Instructions—In Process Check) page 520F may correspond to an approved study Instructions and Equipment. As shown in the figure, the Instructions and Equipment (Study Instructions—In Process Check) page 520F can include the plurality of navigational user affordances 522, the Instruction-Type navigational user affordances 542F, and Instructions and Equipment (Study Instructions—In Process Check) display information 526F. As shown in the figure, the Instructions and Equipment (Study Instructions—In Process Check) display information 526F can include, but is not limited to, the sequence, text, response type, job type, instruction set name, instruction set description, and version.

In one or more examples, the in-process check can be provided in multiple languages. In some examples, the translations may depend on the packaging sites associated with the study batch record 108 and/or study part 106. In such examples, the text associated with the in-process check may be translated based on the English in-process check to the selected language. For example, a German translation can be created based on the English in-process check.

FIG. 5G illustrates an exemplary Instructions and Equipment (Study Instructions—Important Notes) page 520G from the Study Batch Record UI. The important notes may correspond to notes to the operator to adhere to while the product is being packaged. Instructions and Equipment (Study Instructions—Important Notes) page 520G may correspond to an approved study Instructions and Equipment. As shown in the figure, the Instructions and Equipment (Study Instructions—Important Notes) page 520G can include the plurality of navigational user affordances 522, the Instruction-Type navigational user affordances 542G, and Instructions and Equipment (Study Instructions—Important Notes) display information 526G. As shown in the figure, the Instructions and Equipment (Study Instructions—Important Notes) display information 526G can include, but is not limited to, the text of the important notes.

In one or more examples, the important notes can be provided in multiple languages. In some examples, the translations may depend on the packaging sites associated with the study batch record 108 and/or study part 106. In such examples, the text associated with the important notes may be translated based on the English important notes to the selected language. For example, a German translation can be created based on the English important notes.

In one or more examples, the system can allow a user to edit the study instructions. FIG. 5H illustrates an exemplary Instructions and Equipment (Study Instructions—Packaging Instructions) page 520H according to embodiments of the present disclosure. Instructions and Equipment (Study Instructions—Packaging Instructions) page 520H can correspond to a draft Instructions and Equipment (e.g., unapproved). In one or more examples, a user can navigate to the Instructions and Equipment (Study Instructions—Packaging Instructions) page 520H using the plurality of navigational user affordances 522 and/or or the second plurality of navigational affordances 542. The system can receive a selection of an Edit Instructions user affordance 554H from the user.

Upon receiving the selection of the Edit Instructions user affordance 554H, the system can display Edit Packaging Instructions Window 530I. As shown in the figure, the Edit Packaging Instructions Window 530I can include a plurality of user affordances to edit details associated with the packaging instructions. For example, the user affordances can include text entry boxes, drop down menus, and the like. In one or more examples, the instruction name, text, description, and one or more images, can be edited via Edit Packaging Instructions Window 530I. In one or more examples, once packaging instructions and/or equipment groups are approved, the system may not permit the packaging instructions to be edited.

In this manner, a central repository for packaging instructions, in process check, and important notes can be created and maintained by the system. In one or more examples, the instructions can be accessed and maintained across various locations. For example, the instructions can be accessed by the company running the clinical trial study (e.g., customer), the production facilitator, and at the packaging site. In contrast, packaging instructions for clinical trial studies typically vary by packaging location. This can lead to issues if changes are made to instructions at one packaging site and not another, and can affect the consistency and uniformity of the output materials across packaging sites.

At Block 506, the system can receive an indication to add equipment groups to the study batch record. The equipment groups can correspond to one or more types of equipment that may be used to at the packaging site to package the output material. For example, if the output material corresponds to a bottle of tablets, a bottle sealer may be used to package and seal the bottle.

In one or more examples, the system can receive an indication that a user has selected the Instructions and Equipment Group tab from the navigational user affordances 522. Upon receiving the selection of the Equipment Group tab, the system can navigate to the Instructions and Equipment Group (Equipment Groups) page 520J, as shown in FIG. 5J. Instructions and Equipment Group (Equipment Groups) page 520J corresponds to a draft Study Batch Record UI (e.g., not an approved study batch record). The system can receive a selection of the New Equipment Group user affordance 556J, which can cause a New Equipment Group Window. In one or more examples, the equipment groups can be edited using the edit-equipment group user affordance 558J, as shown in FIG. 5J.

FIG. 5K illustrates an exemplary New Equipment Group Window 530K in accordance with embodiments of the present disclosure. As shown in the figure, the New Equipment Group Window 530K can includes a plurality of input user affordances for a user to enter details regarding the equipment group. As shown in the figure, the user affordances can include text entry boxes, drop down menus, and the like. In one or more examples, system can obtain an equipment group and equipment details.

FIG. 5L illustrates an exemplary approved Instructions and Equipment Group (Equipment Groups) page 520L. As shown in the figure, the Instructions and Equipment Group (Equipment Groups) page 520L can include a plurality of navigational affordances 522 and the Instructions and Equipment Group (Equipment Groups) information display region 526L. As shown in the figure, and the Instructions and Equipment Group (Equipment Groups) information display region 526L can include a list of equipment groups and corresponding equipment details associated with the study batch record.

At Block 508, the system can receive an approval of one or more instruction sets and equipment groups from the one or more approvers. In one or more examples, the system will not permit an approval of the instructions and equipment groups to be accessed without receiving completed destination instructions and equipment groups' information. In one or more examples, the process associated with Block 508 can be similar to Block 312 of process 354.

In one or more examples, the system can receive an indication that a user has selected the Approval button from the Study Batch Record operational user affordances 524. Upon receiving the selection of the Approval button, the system can present an approval report 572M. The approval report can include the packaging instructions and equipment groups' information associated with each of the Information Display regions. For example, the approval report 572M can include the information included in one or more of the Study Batch Record information display region 526A, Instructions and Equipment (Study Instructions—Packaging Instructions) display information 526E, Instructions and Equipment (Study Instructions—In Process Check) display information 526F, Instructions and Equipment (Study Instructions—Important Notes) display information 526G, Instructions and Equipment Group (Equipment Groups) information display region 526L.

Once a user is ready to sign the Approval Report 572M, e.g., after the user has reviewed and verified the information in the Approval Report 572M, the system can receive an indication that the production user has selected the signature button 576M. Upon receiving the signature button, the system can cause a signature window 574N to appear. In one or more examples, the signature window can be overlaid on the Approval Report 572M. The signature window 574N can include one or more user affordances for receiving the credentials of the production user. For example, the signature window 574N can include a textbox for a production user to input their userID and corresponding password. The system can then receive an indication of the user accepting or rejecting the approval.

In one or more examples, once the packaging instructions and equipment groups are approved, the system may not permit changes to be made to the approved version of the packaging instructions and equipment groups (e.g., packaging instructions 110). If a user desires to make changes to packaging instructions 110, then the production user can create a new version of the packaging instructions 110. The system can create a new version of the packaging instructions 110 by receiving a selection by the user of the New Version button from the operational user affordances 524. Changes to the new version of the packaging instructions 110 can be made in accordance with the description provided above, e.g., with respect to process 558. Once a new version is created, the previous version will remain the controlling version of the study information until the new version is approved, as described above with respect to Block 508.

In one or more examples, the packaging instructions can be approved separately for each language and/or translation. For example, if packaging instructions for a study batch record are in English, German, and Chinese, then a separate approval for each language should be executed. For example, referring briefly to Study Batch Record Details page 520, a separate plurality of operational user affordances 538 are provide with respect to the German instructions. In this manner, translations for the instructions can be reviewed and approved separately. In one or more examples, the English instructions should be approved prior to approving packaging instructions for other languages.

In one or more examples, there may arise a situation where a version 1 of a study part includes English as a site language. Accordingly, the generated study instructions may have an English header. Thus, the English instructions can be created and/or imported. In such an example, version 2 of the study part can be created and approved. In this example, version 2 can include both English and German as the site languages. In such examples, when the user versions up the Study Instructions, the system can search for the latest approved study part. The system can determine that English and German should correspond to the site languages. Accordingly, the system can create headers for the new study instruction version in both English and German. However, there are no German Instructions (e.g., because German was not previously included in the site language). In such examples, the system can clone the English instructions to create the German instructions. In such examples, the user may have to translate the German instructions before submitting for Approval.

In one or more examples, a production user can copy study instructions from a previous draft and/or other approved study instruction belonging to a study linked to the same customer. In such examples, if there is a missing language (e.g., as described above regarding versioning), the system can copy the English instructions to create the corresponding translations for various other site instruction languages. In one or more examples, extra languages in the selected Study Instructions that are not present in the original may be ignored.

At Block 510, the system can obtain information corresponding to the bill of materials. In one or more examples, information obtained from the Output and Input Materials page, e.g., Output and Input Materials page 420D, created in the study part 108 (e.g., Blocks 404-412 of FIG. 4A) can be carried over to the bill of materials. For example, the system can receive at least an item ID 5820 and quantity 5840 for each of the materials from the study part 108. In one or more examples, reconciliation limits can be configured for each of the input materials (e.g., drug, non-drug, and label). For example, the production user can select reconciliation limits from a drop-down list of reconciliation units.

FIG. 5P illustrates an exemplary Bill of Materials (Output Material-Input Material) page 520P (Output Material-Input Material) from the Study Batch Records UI. In one or more examples, the Bill of Materials (Output Material-Input Material) page 520P can include the plurality of navigational user affordances 522, the plurality of operational user affordances 524, and a Bill of Materials (Output Material-Input Material) information display region 526P. The Bill of Materials (Output Material-Input Material) information display region 526P can include information related to the approval status, the version number, the bill of materials identifier, a hold reason, a reason for versioning, a URL, output material information (e.g., item identifier, one or more material descriptions, an inventory number, a label country group, an output material group, a dosage code), drug input material information (e.g., item identifier, one or more material descriptions, an inventory number, a label, an alternative material, and a quantity), a non-drug input material information (e.g., item identifier, one or more material descriptions, an inventory number, a label, an alternative material, and a quantity), and a label input material information (e.g., item identifier, one or more material descriptions, an inventory number, a label, an alternative material, and a quantity).

FIG. 5Q illustrates an exemplary Bill of Materials (Common Materials) page 520Q from the Study Batch Records UI. In one or more examples, the Bill of Materials (Common Materials) page 520Q can include the plurality of navigational user affordances 522, the plurality of operational user affordances 524, and a Bill of Materials (Common Materials) information display region 526Q. The Bill of Materials (Common Materials) information display region 526Q can include information related to the approval status, the version number, the bill of materials identifier, a hold reason, a reason for versioning, a URL, a common-drug material information (e.g., item identifier, one or more material descriptions, an inventory number, a label country group, an output material group, a dosage code), a common-non-drug input material information (e.g., item identifier, one or more material descriptions, an inventory number, a label, an alternative material, and a quantity), and a common-label input material information (e.g., item identifier, one or more material descriptions, an inventory number, a label, an alternative material, and a quantity).

In one or more examples, the system can obtain information related to the bill of materials from third party software. For example, the system may be integrated with a third party software (e.g., JD Edwards) that can provide item identifiers associated with an inventory maintained by the third party software. In one or more examples, the production user can select one or more item identifiers based on the integration with the third party software. FIG. 5R illustrates an exemplary portion of a Study Batch Record page 520R according to embodiments of this disclosure. As shown in the figure, the system includes a plurality of operational user affordances, including “Get Item IDs” user affordance 584R. Upon receiving a selection of the user affordance 584R, the system can present a pop-up window to the user. FIG. 5S illustrates an exemplary pop-up window according to embodiments of this disclosure. The pop-up window 530S can include an inventory associated with and/or maintained by the third party software that includes a list of item identifiers (and corresponding descriptions) that can be assigned to input and/or output materials in the study batch record. In one or more examples, a user can select one or more of the list items and update the study batch record accordingly.

In one or more examples, example packs can be included in the bill of materials. FIG. 5T illustrates an exemplary Example Pack Window 530T. The Example Pack Window 520T can be configured to obtain information from a production user regarding the example pack. The information can include general information regarding the example pack (e.g., material description, bill of materials quantity, UoM information, bill of materials information, customer material identifier), and customer inventory information (e.g., item identifier, one or more item descriptions, and third party software information). In one or more examples, the bill of materials quantity may automatically be set to one.

At Block 512, the system can receive an approval of the bill of materials from the one or more production approvers. In one or more examples, the system may not permit an approval of the bill of materials to be accessed without receiving completed bill of materials. In one or more examples, the process associated with Block 512 can be similar to Block 508.

For example, the system can receive an indication that a user has navigated to the Bill of Materials page, e.g., 520P associated with the Study Batch UI. FIG. 5U illustrates an exemplary portion of a Bill of Materials page 520P that includes an approval user affordance 578U. In one or more examples, the system can receive an indication that the user has selected the approval user affordance 578U from the operational user affordances 524. Upon receiving the selection of the approval user affordance 578U, the system can present an approval report 572V. The approval report can include the information associated with the bill of materials information display regions, e.g., Output Material-Input Material display region 526P, Common Material information display region 526Q.

Once a production user is ready to sign the Approval Report 572V, e.g., after the user has reviewed and verified the information in the Approval Report 572V, the system can receive an indication that the user has selected the signature button 576V. Upon receiving an input corresponding to a selection of the signature button the system can cause a signature window 574W to appear. In one or more examples, the signature window can be overlaid on the Approval Report 572V. The signature window 574W can include one or more user affordances for receiving the credentials of the user. For example, the signature window 574W can include a textbox for a production user to input their userID and corresponding password. The system can then receive an indication of the user accepting or rejecting the approval.

In one or more examples, once the bill of material are approved, the system may not permit changes to be made to the approved version of the bill of materials (e.g., bill of materials 112). If a production user desires to make changes to bill of materials 112, then the user can create a new version of the bill of materials 112. The system can create a new version of the bill of materials 112 by receiving a selection by the production user of the New Version button from the operational user affordances 524. Changes to the new version of the bill of materials 112 can be made in accordance with the description provided above, e.g., with respect to process 558.

In one or more examples, the system can present the Related Study Parts page. FIG. 5X illustrates an exemplary Related Study Parts page 520X from the Study Batch Records UI that is configured to show a list of study parts associated with the same study information. In one or more examples, the Related Study Parts page 520X can include the plurality of navigational user affordances 522, the plurality of operational user affordances 524, and a Related Study Parts information display region 526X. The Related Study Parts information display region 526X can include a list and/or links to the study parts that are associated with the same study information, e.g., study information 104.

In one or more examples, the system can present the Related Study Components page. FIG. 5Y illustrates an exemplary Related Study Components page 520Y from the Study Batch Records UI, which is configured to show the related study components (e.g., study instructions, bill of materials, and study batch records) associated with the same study information. In one or more examples, the Related Study Components page 520Y can include the plurality of navigational user affordances 522, the plurality of operational user affordances 524, and a Related Study Components information display region 526Y. The Related Study Components information display region 526Y can include a list of the related study components, e.g., study instructions, bill of materials, and related batch records.

### Creating Instructions

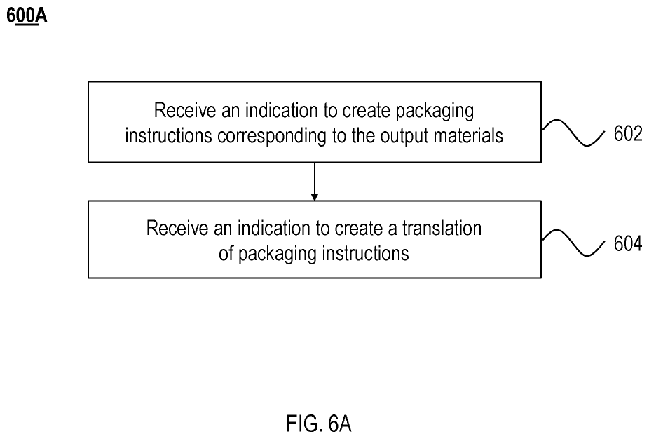

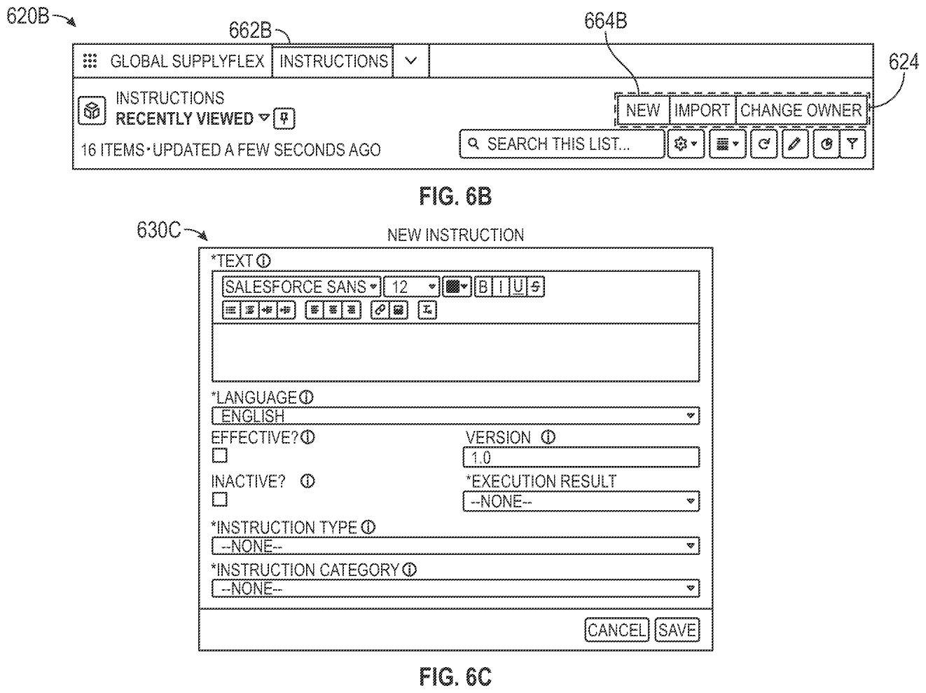

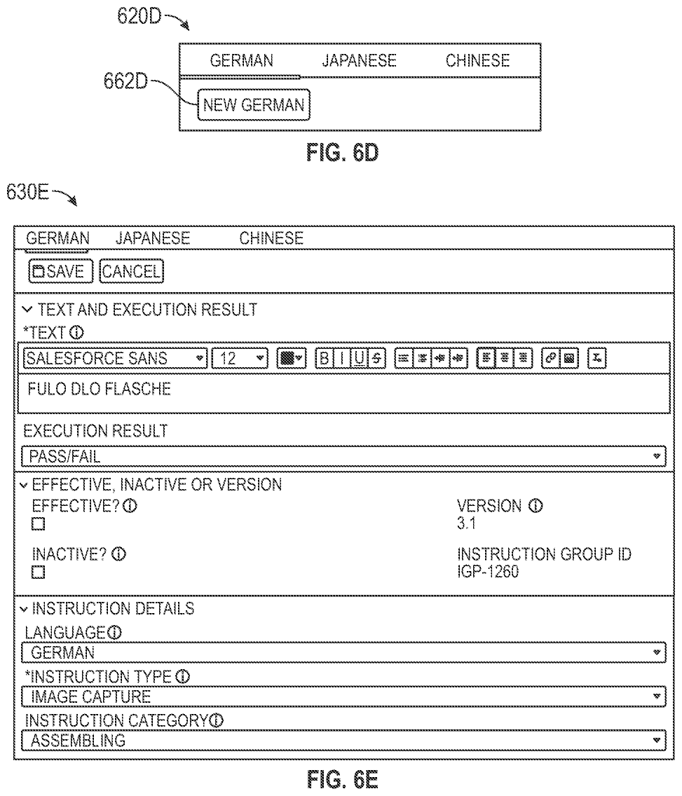

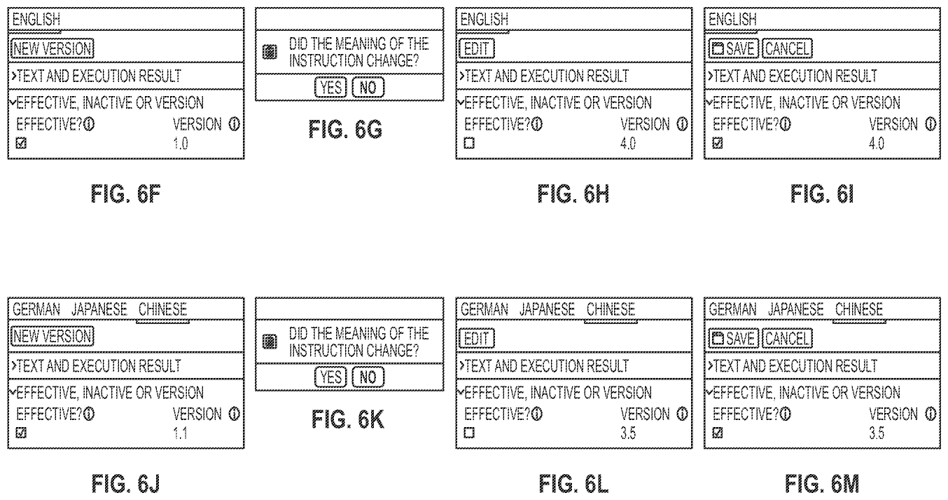

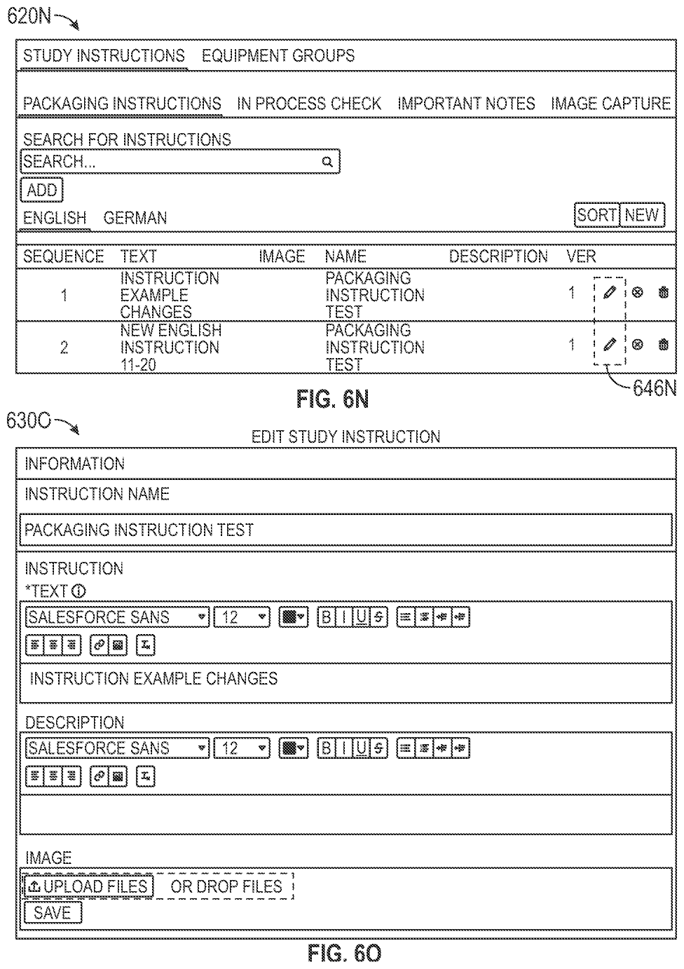

FIG. 6A illustrates a process 600A for creating packaging instructions according to one or more embodiments of this disclosures. At Block 602, the system can receive an indication to create a set of instructions corresponding to the output materials. The instructions can correspond to packing instructions for the output materials. For example, the system can receive an indication to navigate to the instructions page 220H, corresponding to packaging instructions 128 from master data 102. For example, a user can select the “Instructions” label from a navigation menu 662. FIG. 6B illustrates an exemplary portion of a Packaging Instructions page 620B according to embodiments of this disclosure. As shown in the figure, the system includes a plurality of operational user affordances 624, including a new instruction user affordance 664B. Upon receiving a selection of the new instruction user affordance 664B, the system can present a New Instruction Window to the user. FIG. 6C illustrates an exemplary New Instruction Window 630C according to embodiments of this disclosure. The New Instruction Window 630C can include text entry boxes, drop down menus, and the like. In one or more embodiments, the Instruction Window 630C can be configured to obtain the packaging instructions text, language, an effective status, a version number, an execution result, an instruction type, and an instruction category.

At Block 604, the system can receive an indication to translate the instructions. For example, the system can navigate to a language tab corresponding to an available language for the instructions. In one or more examples, the available language may be based on one or more packaging sites associated with the customer. FIG. 6D illustrates an exemplary portion of the Instructions page 620D. As shown in the figure, upon receiving a selection of a desired language tab from the production user, the system can present a new translation user affordance 662D. Upon receiving a selection of the new translation user affordance 662D, the system can present a New Translation Window 630E. The New Translation Window 630E can include a plurality of user affordances for generating the translation, including, a text formatting affordance, an execution result affordance, an effective instruction user affordance, an inactive instruction user affordance, and instruction details (e.g., language, instruction type, and instruction category). Upon completion of the fields, the system can generate the translation of the instructions.

In one or more examples, when an Instruction is marked effective, the instruction can be used in Instruction Sets and Study Instructions. Changes cannot be made to effective instructions. In order to make changes to effective instructions, a new version of the instruction must be created. In one or more examples, new versions can be based on effective instructions. Accordingly, to update effective instructions, the system can receive an indication that a new version of the instructions should be created as shown in FIG. 6F.

As shown in FIG. 6G, the system can then receive an indication of whether the meaning of the instructions has changed. As shown in FIG. 6H, the system can then receive an indication of whether the instructions are effective. In one or more examples, as shown in FIG. 6I, a user can manually update translations based on the English instructions when a new version of the English instructions are created.

In one or more examples, the system can also update an effective translation of packaging instructions. In such examples, as shown in FIG. 6J, the system can receive an indication that a new version of the translation of the packaging instructions should be created. As shown in FIG. 6K, the system can receive an indication of whether the meaning of the instructions has changed. As shown in FIG. 6L, the system can then receive an indication that the translation should be edited. As shown in FIG. 6M, the system can receive an indication of whether the instructions are effective.

English Instructions can also be made Inactive, which can restrict users from adding the instruction 110 to a study batch record 108. When the user changes the effective instructions back to active, the system can update all related translations to active. Instructions that are not used within instruction sets 130 or study instructions 110 can be deleted.

In one or more examples, instructions created at Block 602 can be edited. FIG. 6N illustrates an exemplary portion of a Study Instructions (Packaging Instructions) page 620N that shows instruction-editing user affordances 646N. Upon receiving a selection of an instruction-editing user affordance 646N, the system can present an Instruction-Editing Window. FIG. 6O illustrates an exemplary Instruction-Editing Window 630O. Instruction-Editing Window 630O can be similar to New Instruction Window 630C, and can be updated as described above with respect to New Instruction Window 630C.

### Lot Assignment

In one or more examples, the lot assignment can be used to associate a batch record with a particular packaging site and quantity of output materials. For example, the study batch record 108, e.g., the bill of materials 112, may indicate the output material(s), e.g., a 60-count bottle including 60 tablets, while the lot assignment will specify that 100 of these bottles should be included in a particular batch at a particular packaging site. In one or more examples, the Production Facilitator UI can present a list of the approved bill of materials (e.g., each bill of materials corresponding to a respective study part) to a production user. The Production Facilitator UI can then receive an indication from the production user of one or more bills of materials should be included in the lot assignment and the site(s) where the output materials are to be packaged. In this manner, the system can determine the output materials and the quantity of these materials to be produced and where the output materials are to be produced.

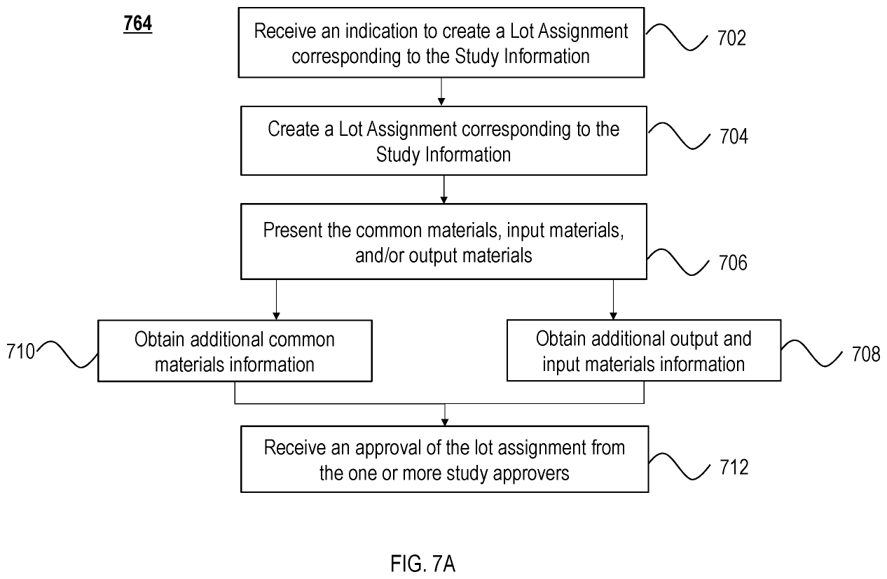

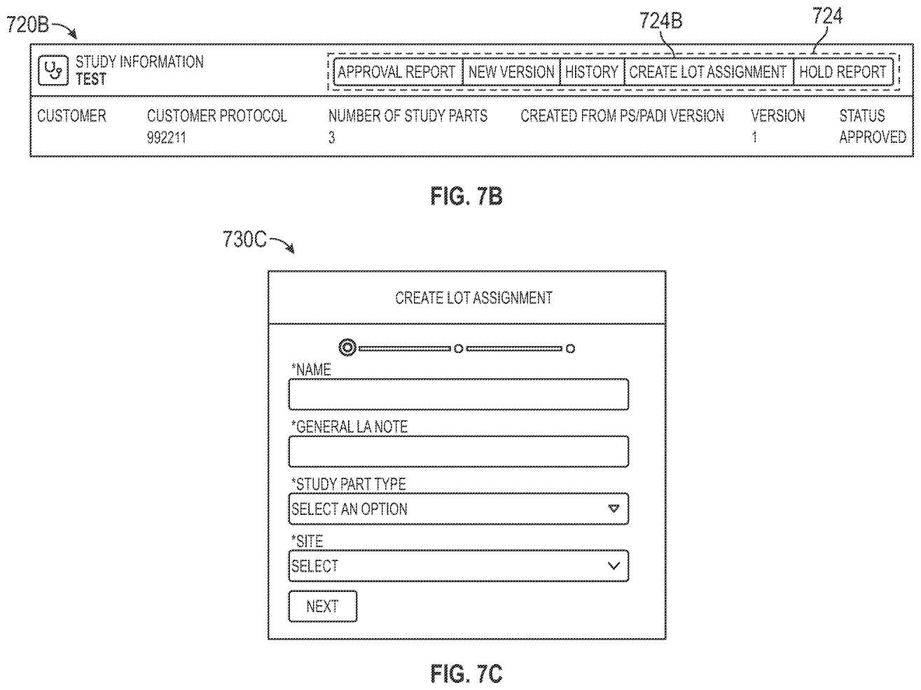

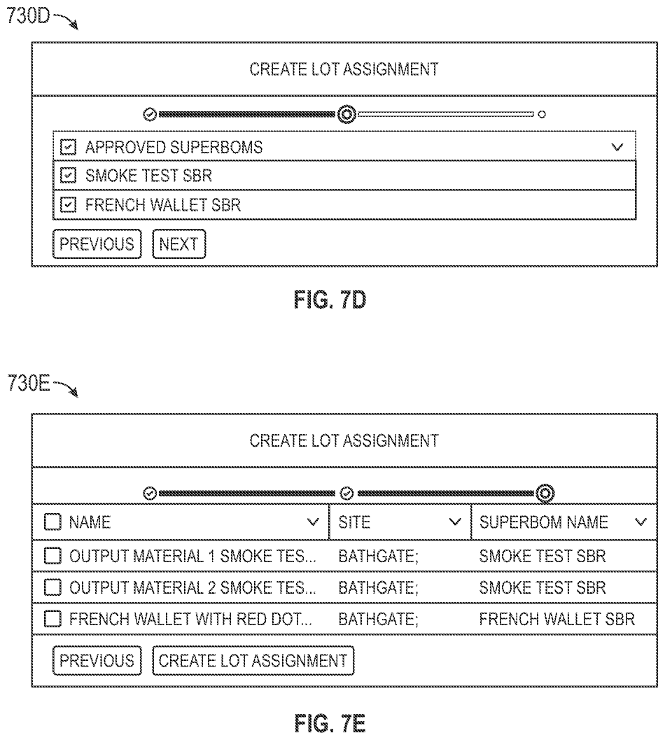

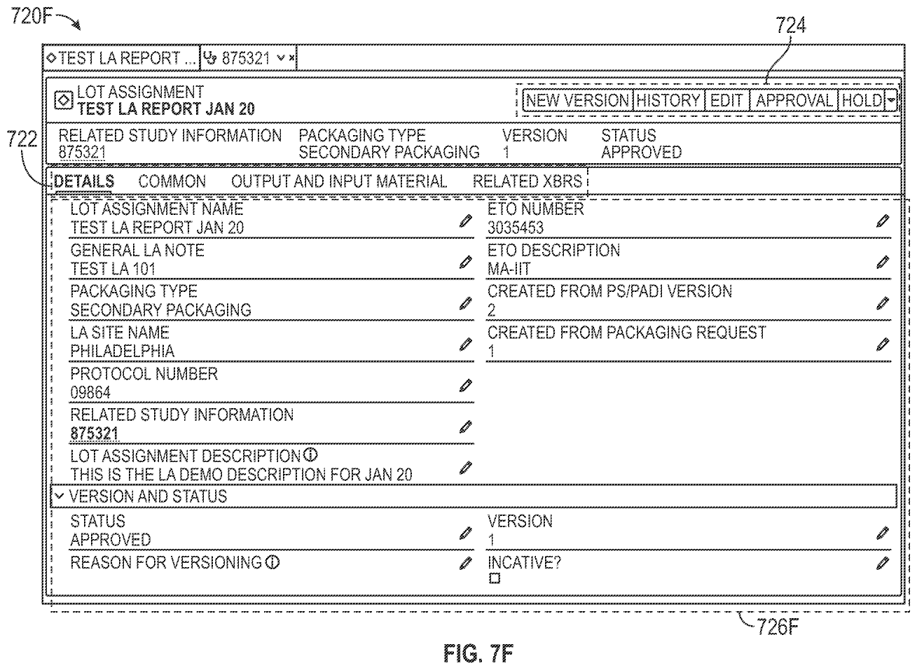

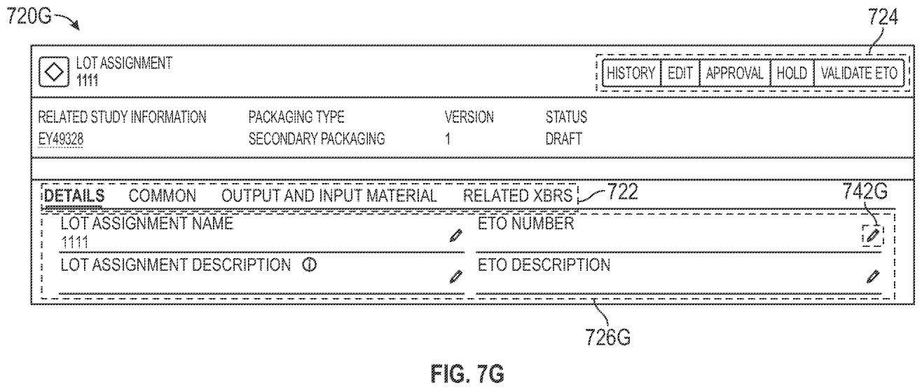

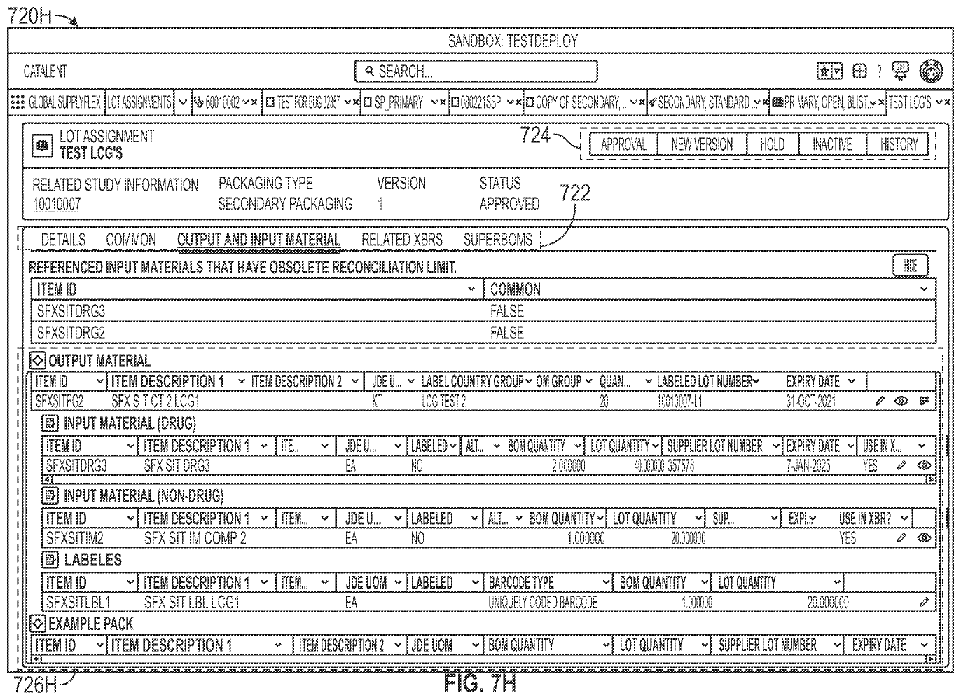

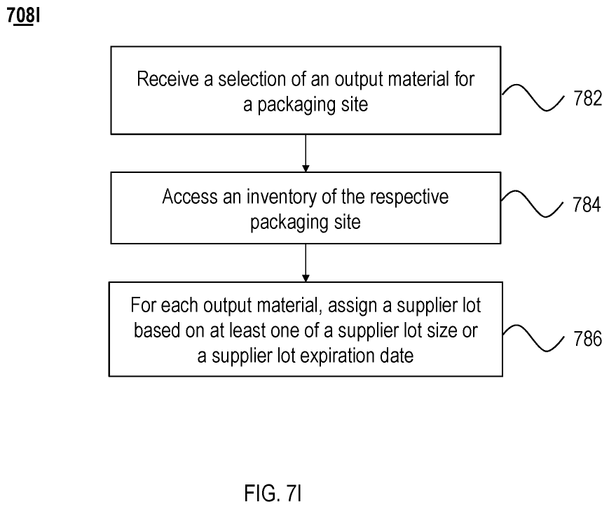

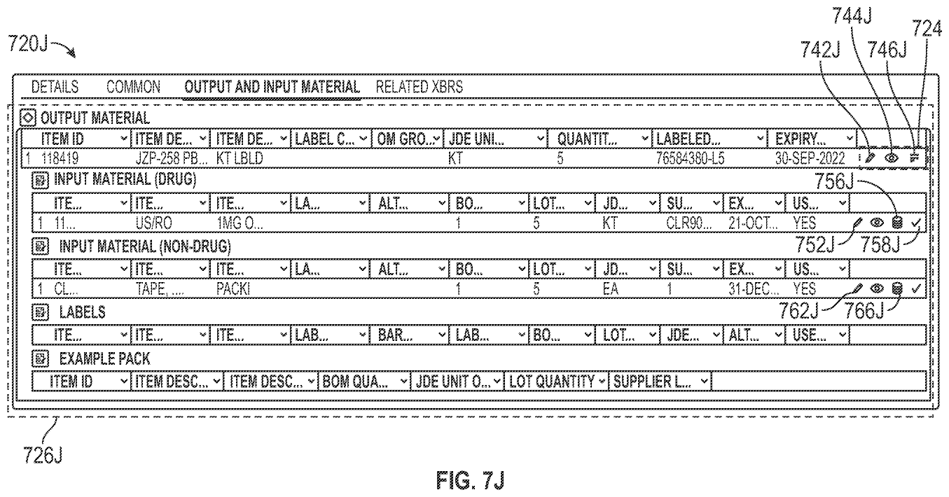

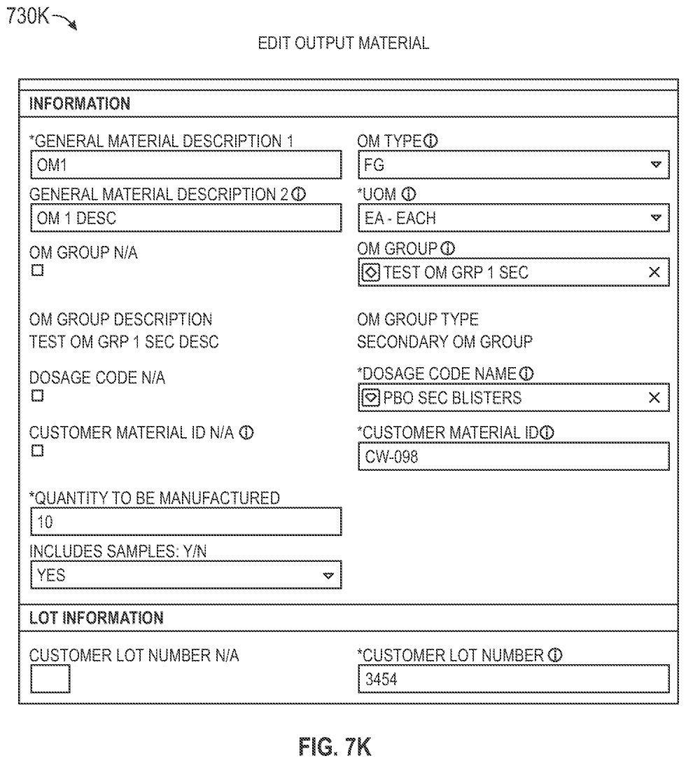

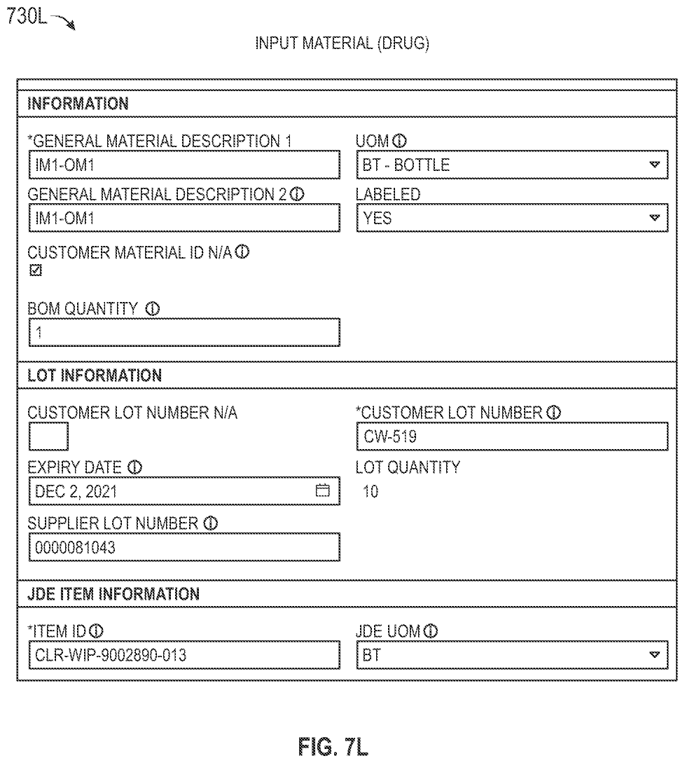

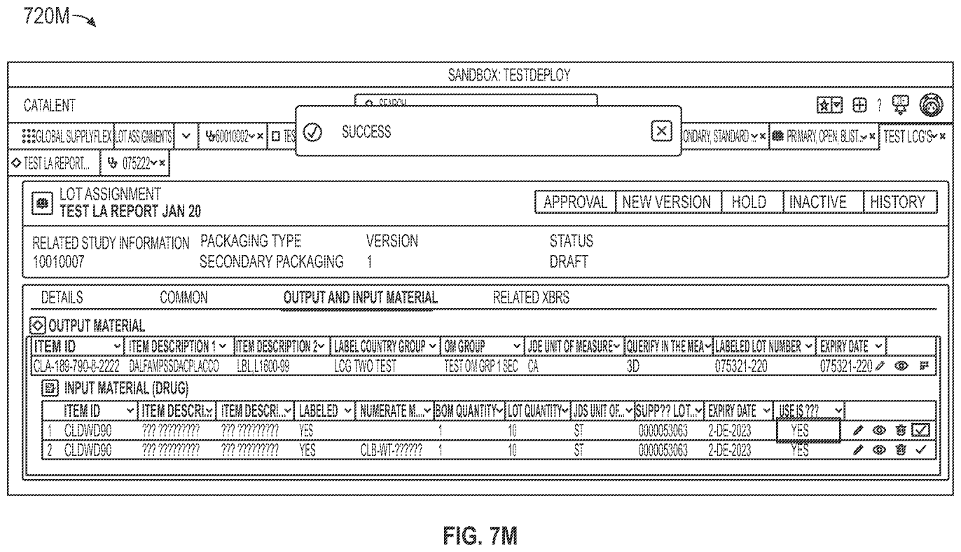

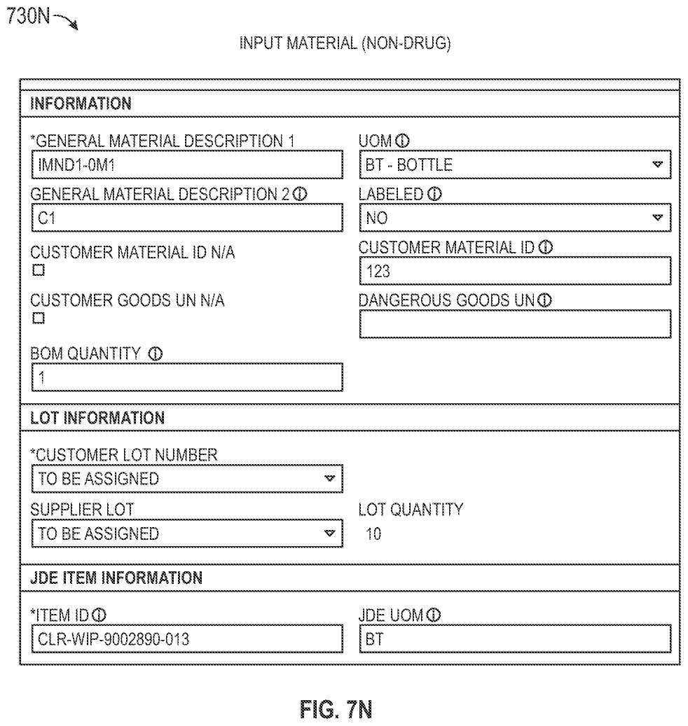

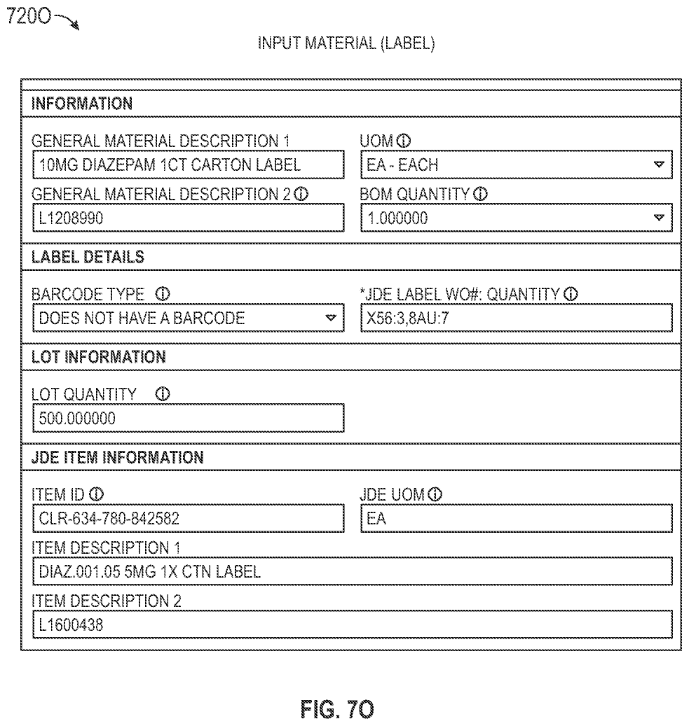

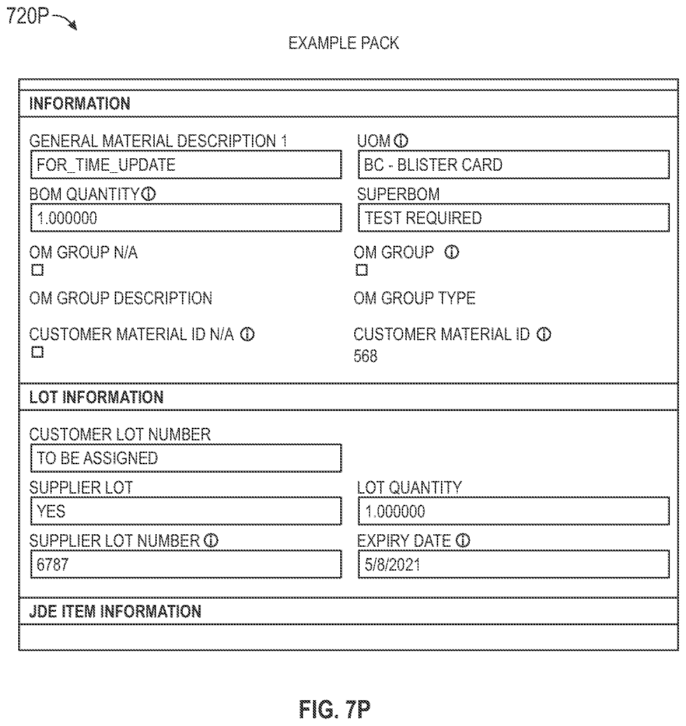

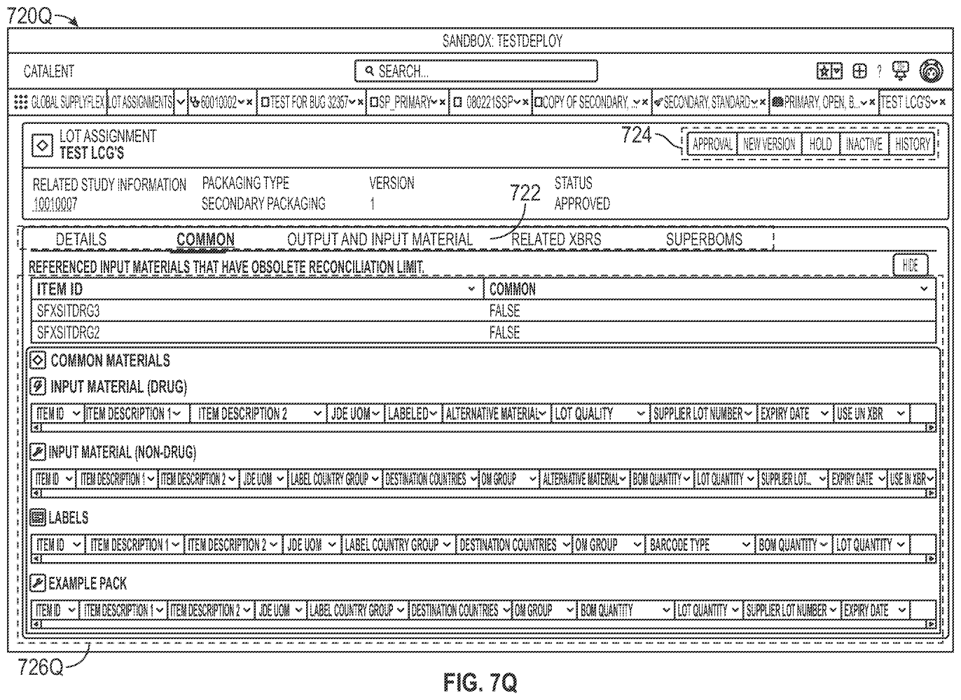

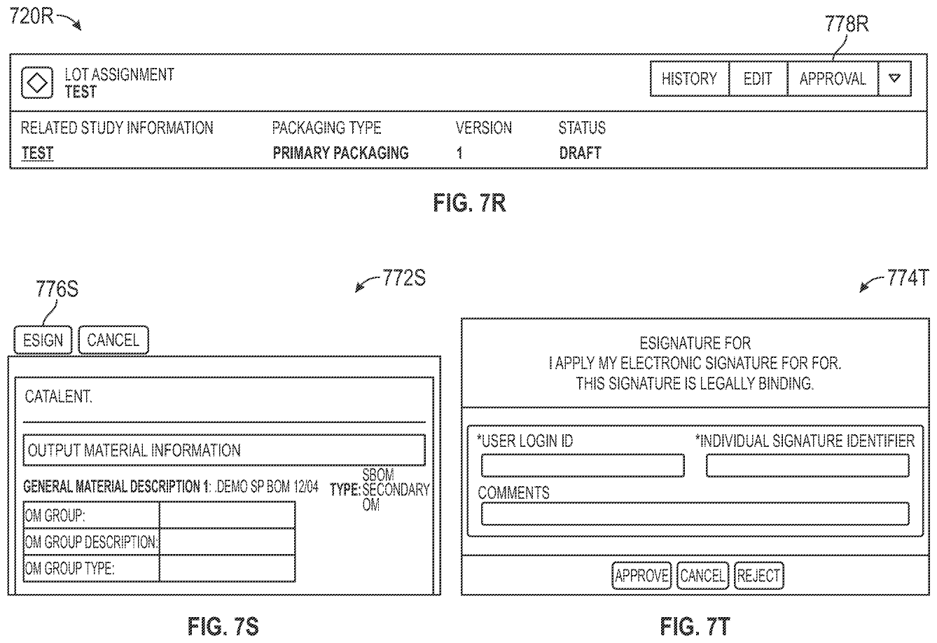

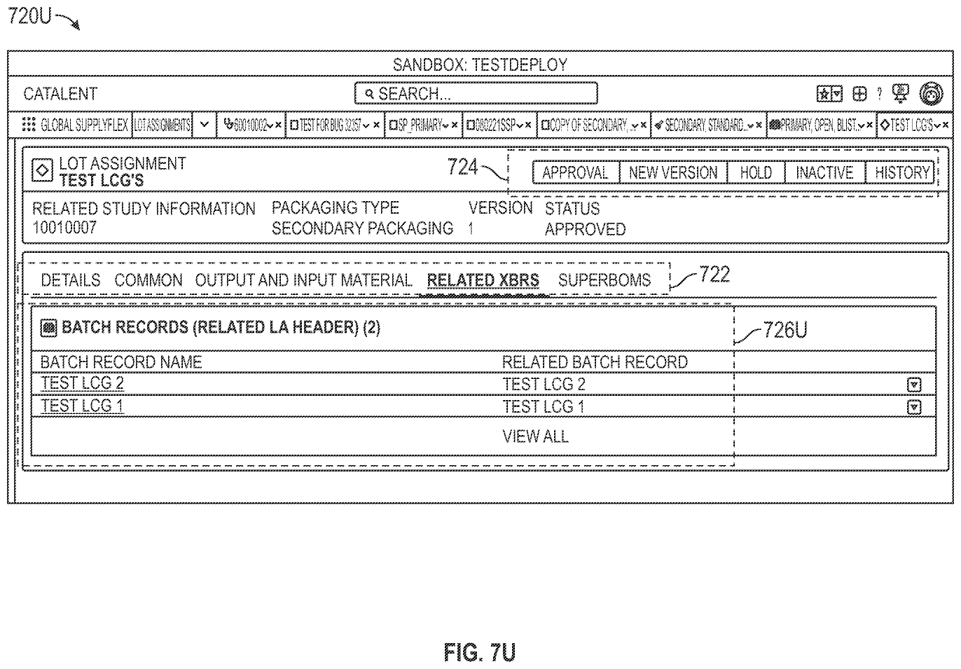

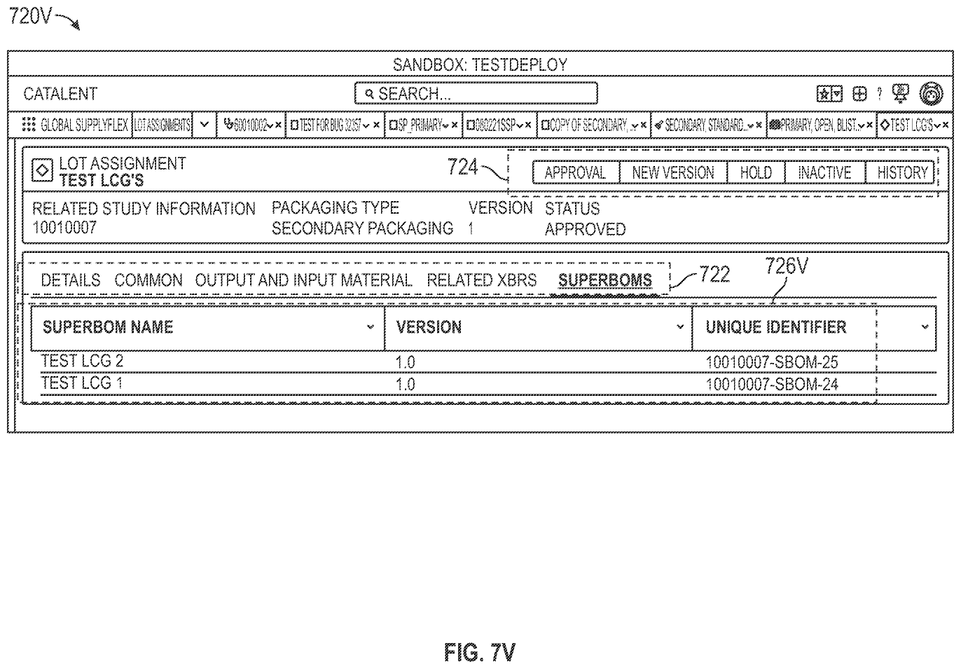

FIG. 7A illustrates process 764 for obtaining a lot assignment 114, according to one or more embodiments of the present disclosure. In one or more embodiments, process 764 can correspond to Block 164 discussed above with respect to process 150 and process 160. Process 764 can be performed, for example, using one or more electronic devices implementing a software platform. In some examples, process 764 is performed using a client-server system, and the blocks of process 764 are divided up in any manner between the server and a client device. In other examples, the blocks of process 764 are divided up between the server and multiple client devices. In other examples, process 764 is performed using only a client device or only multiple client devices. In process 764, some blocks are, optionally, combined, the order of some blocks is, optionally, changed, and some blocks are, optionally, omitted. In some examples, additional steps may be performed in combination with the process 764. Accordingly, the operations as illustrated (and described in greater detail below) are exemplary by nature and, as such, should not be viewed as limiting.

At Block 702, the system can receive an indication to create a lot assignment 114 associated with the study information 102. For example, while the Production Facilitator UI can receive an indication to create a lot assignment record from the Study Information UI. FIG. 7B illustrates a portion of a Study Information page 720B, that includes a plurality of operational affordances 724, including a “Create Lot Assignment” user affordance 742B. In one or more examples, the “Create Lot Assignment” user affordance 742B may become visible upon approval of the bill of materials and packaging instructions.

Upon receiving the selection of the create lot assignment user affordance 742B, the system can present the Create Lot Assignment Windows 730C, 730D, and 730E, as shown in FIGS. 7C-7E. The Create Lot Assignment Windows 730C-730E can include a plurality of user affordances to obtain details regarding the Lot Assignment from a production user. As shown in the figure, the user affordances can include text entry boxes, drop down menus, checkboxes, and the like. In one or more examples, the Lot Assignment Window 730C can include user affordances for obtaining information regarding a lot assignment name, lot assignment note, study part type (e.g., primary, secondary), packaging site location. In one or more examples, the Lot Assignment Window 730D can include user affordances for selecting previously approved bill of materials (e.g., corresponding to one or more bills of materials that were approved via process 658). In one or more examples, the Lot Assignment Window 730E can include user affordances for output materials associated with to the packaging site selected in Lot Assignment Window 730C.

At Block 704, the system can create a lot assignment record corresponding to the study information. FIG. 7F illustrates an exemplary Lot Assignment Details page 720F corresponding to a Lot Assignment UI created based on the completion of Create Lot Assignment Window 730C-730E. The Lot Assignment Details page can be presented via the Production Facilitator UI and configured to display information related to the lot assignment record. As shown in the figure, the Lot Assignment Details page 720F can include a plurality of navigational user affordances 722, a plurality of operational user affordances 724, and a Lot Assignment Detail information display region 726F. In one or more examples, the Lot Assignment Detail information display region 726F can correspond to an area of the Lot Assignment UI that displays the information regarding the lot assignment. As shown in the figure, the Lot Assignment Detail information display region 726F can display information included, but not limited to, a lot assignment name, a lot assignment note, a packaging type, a lot assignment site location, a protocol number, a related study information number, a lot assignment description, an approval status, an active/inactive status, and a version number.

In one or more examples, the Study Parts navigational user affordances 722 can include a plurality of user affordances, where each user affordance corresponds to a tab associated with the study information. As shown in the figure, the Study Parts navigational user affordances 722 can include a Lot Assignment Detail tab, an Output and Input Material tab, a Common Material Tab, and a Related Batch Records tab. The examples of navigational user affordances 722 are merely exemplary and more or less navigational user affordances can be used without departing from the scope of this disclosure.

In one or more examples, the plurality of operational user affordances 724 can include a plurality of buttons that each correspond to a different action that can be implemented by a user. As shown in the figure, the operational user affordances 724 can include, but is not limited to an approval button, a new version button, an edit button, a hold button, a history button, and the like. In one or more embodiments, depending on the status of different items associated with study part 106, one or more of the operational user affordances 724 may be inactive and/or one or more of the operational user affordances may be removed or made visible. The operational user affordances can have functions similar to those described above.

In one or more examples, once the lot assignment 114 is created, the system can receive a confirmation of the engineering-to-order (ETO) number and ETO description. FIG. 7G illustrates an exemplary Lot Assignment Details page 720G, according to embodiments of this disclosure. Lot Assignment Details page 720G may correspond to a draft lot assignment. In one or more examples, the system can receive an indication that a user has selected an edit-ETO number user affordance 742G. Upon receiving selection of the edit-ETO number user affordance 742G, the system can receive an entry of the ETO number from the user. The system can then access information regarding the ETO and apply the ETO information to the lot assignment.

Once the lot assignment record is created and in a draft status production users can update the common materials, output materials and input materials. In one or more examples, the common materials, output materials and input materials can be auto-populated based on information associated with the bill of materials 112 approved in process 558 and/or at Block 158. Referring back to FIG. 7A, at Block 706, the system can present the common materials, input materials and/or output materials for review. For example, a user can navigate to a Common Materials page or an Output and Input Materials page using a corresponding navigational user affordance 722.

FIG. 7H illustrates an exemplary Output and Input Materials page 720H according to embodiments of the present disclosure. In one or more examples, the Output and Input Materials page 720H can include multiple output materials. Output and Input Materials page 720H can correspond to an approved Output and Input Materials page 720H. As shown in the figure, the Output and Input Materials page 720H can include the navigational user affordance 722, the operational user affordance 724, and the Output and Input Materials information display region 726H. In one or more examples, the Output and Input Materials information display region 726H can include, but is not limited to, a plurality of user affordances, information regarding output materials (e.g., an item identifier, one or more item descriptions, a UoM, a label country group, an OM group, a label lot number and an expiry date), drug input materials (e.g., an item identifier, one or more item descriptions, a UoM, a label status, an alternative material, a bill of materials quantity, a lot quantity, a supplier lot number, an expiry date, and a batch record status), non-drug input materials (e.g., an item identifier, one or more item descriptions, a UoM, a label status, an alternative material, a bill of materials quantity, a lot quantity, a supplier lot number, an expiry date, and a batch record status), label input materials (e.g., an item identifier, one or more item descriptions, a UoM, a label status, a barcode type, a bill of materials quantity, a lot quantity, a supplier lot number, an expiry date, and a batch record status), and example pack materials (e.g., an item identifier, one or more item descriptions, a UoM, a label status, an alternative material, a bill of materials quantity, a lot quantity, a supplier lot number, an expiry date, and a batch record status). In one or more examples, information regarding the output and input materials may be auto-populated based on the corresponding study batch record.

FIG. 7I illustrates an exemplary process 708I for obtaining additional output materials information in accordance with embodiments of the present disclosure. In one or more examples, process 708I can correspond to Block 708 of process 764. While process 708I is described with respect to output materials, a similar process can be applied to obtaining additional information regarding input materials (drug, non-drug, label), example pack materials, and common materials.

At Block 782, a user can receive a selection of an output material for a packaging site. In one or more examples, the system can receive a selection by the user to edit the output materials shown on Output and Input Materials page. FIG. 7J illustrates an exemplary Output and Input Materials page 720J according to embodiments of the present disclosure. Output and Input Materials page 720J may be associated with a draft lot assignment, but otherwise be substantially similar to Output and Input Materials page 720H. As shown in the figure, a user can select one or more user affordances 724 to update the output material. For example, the user affordances 724 can be used to edit (user affordance 742J) preview (user affordance 744J), and review relationships (user affordance 746J) for the output materials. In one or more examples, the system can receive a selection by the user to edit the output materials via user affordance 742J. The edit user affordance 742J can be used to edit information associated with the output materials, the preview user affordance 744J can be used to view the output material lot assignment record without the ability to update the record. The relationship user affordance 746J can be used to view the output materials associated with an output material group and the common materials that are linked to the output material.

Upon receiving a selection of the user affordance 742J, the system can display an Edit Output Material Window 730K, as shown in FIG. 7K. Edit Output Material Window 730K can include a plurality of input user affordances for a production user to enter details regarding the lot assignment record for the output material. As shown in the figure, the user affordances can include text entry boxes, drop down menus, and the like. In one or more examples, the system can obtain additional information related to a quantity to be manufactured, a supplier lot number, a labeled lot number, an expiry date, a labeled expiry date, a customer lot number, and a samples status. In one or more examples, the system may not permit a production user to edit information that was imported from the study batch record.

In one or more examples, the additional information (e.g., a quantity to be manufactured, a supplier lot number, a labeled lot number, an expiry date, a labeled expiry date, a customer lot number, and a samples status) can be auto-populated based on inventory records associated with the packaging site corresponding to the lot assignment record. At Block 784, the system can access an inventory of the respective packaging site. For example, the system can access the inventory of the respective packaging site via integration with a third party software and/or server that maintains the inventory of the respective packaging site. In one or more examples, the inventory information can include information regarding output materials, input materials (drug and non-drug) and corresponding supplier lot numbers, supplier lot sizes, and supplier lot expiration dates associated with a packaging site. In one or more examples, a production user can view and enter one or more of the quantity to be manufactured, a supplier lot number, a labeled lot number, an expiry date, a labeled expiry date, a customer lot number, and a samples status based, in part, on the inventory of the packaging site.

In one or more examples, at Block 786, the system can automatically assign at least a supplier lot number, a labeled lot number, an expiry date, a labeled expiry date, and a customer lot number based on the accessed inventory of the packaging site. For example, the system can automatically assign supplier lot numbers to an input material based on the total quantity of the input material and one or more of the corresponding supplier lot size and/or supplier lot expiration date. In this manner, the system can ensure that there is sufficient inventory at a particular site to package the output materials associated with the batch records, prior to the batch records being sent to the packaging site.

In one or more examples, the system may prioritize supplier lots having the smallest lot size that has the earliest expiration date. For example, if the output quantity indicates that 100 60-count bottles including 60 tablets are to be packaged at a packaging site, the system can access the inventory for the packaging site. Continuing with this example, the packaging site may have five supplier lots corresponding to this output material. According to embodiments of this example, the system can determine a respective supplier lot size and expiration date for each of the supplier lots and assign the supplier lot with the smallest lost size(s) and earliest expiration date(s) to fulfill the 100 60-count bottle output material in the most efficient manner.

In this manner, the system can reduce waste and the amount of human manual labor at the packaging site. For example, typically, a worker at the packaging site would be responsible for reviewing a study batch order and manually looking up the comparing the quantity of the input materials against the inventory available at the packaging site. Not only does this require considerable time and effort, but there is no guarantee that the packaging site will have the necessary inventory to fulfill the study batch record. Accordingly, embodiments of the present disclosure improve on the current state of the art by ensuring that there is sufficient inventory prior to sending the study batch records to a packaging site, reducing waste by assigning supplier lots with earlier expiration dates to be used first, and reducing the amount of manual labor associated with confirming the inventory.

While process 708I is described with respect to output materials, a similar process can be applied to the input materials (drug, non-drug, label, example pack) and common materials.

For example, referring to Output and Input Materials page 720J, the system can receive an indication that a production user selected edit user affordance 752J corresponding to an input (drug) material. Upon receiving a selection of the user affordance 752J, the system can display an Edit Input (Drug) Material Window 730L, as shown in FIG. 7L. Edit Input (Drug) Material Window 730L can include a plurality of input user affordances for a user to enter details regarding the lot assignment record for the input (drug) material. As shown in the figure, the user affordances can include text entry boxes, drop down menus, and the like. In one or more examples, the system can obtain additional information related to a supplier lot number, a lot quantity, an expiry date, and a customer lot number. In one or more examples, the system may not permit a user to edit information that was imported from the study batch record. In one or more examples, the lot quantity can be automatically calculated based on the quantity entered for the corresponding output material.

Referring back to Output and Input Materials page 720J, the system can receive inventory information related to the input (drug) materials. For example, a production user can select a supplier lot user affordance 756J to access inventory records associated with the packaging site. For example, the system can access the inventory of the respective packaging site via integration with a third party software and/or server that maintains the inventory of the respective packaging site. In one or more examples, the inventory information can include information regarding supplier lot numbers, supplier lot sizes, supplier lot status (hold, conditional release, WIP, quarantined, expired, released) and supplier lot expiration dates associated with a packaging site. As discussed above, in one or more examples, the system can automatically assign supplier lot numbers based on the inventory information.

In one or more examples, the system can receive an indication of whether to use the listed input (drug) material or an alternative material. For example, the system can receive an indication that a user selected a material selection user affordance 758J to determine whether the corresponding input (drug) material should be included or an alternative material (e.g., one of the alternative materials designated in the study batch record) should be used in the batch record. For example, if a single input (drug) material record is present under the output material, the lot assignment record will indicate that the input material drug will be used in the batch record. If, however, there are two input (drug) materials, e.g., a main material and an alternative material, the system can receive a selection from the production user to determine which of the two materials should be used in the batch record. In one or more examples, if the main material is selected, the alternative material is automatically de-selected, as shown in FIG. 7M.

FIG. 7N illustrates an Edit Input (Non-Drug) Material Window 730N. In one or more examples, upon receiving a selection of the user affordance 762J from Output and Input Materials page 730J, the system can display the Edit Input (Non-Drug) Material Window 730N, as shown in FIG. 7N. The Edit Input (Non-Drug) Material Window 730N can include a plurality of input user affordances for a production user to enter details regarding the lot assignment record for the input (non-drug) material. The process for updating and editing information associated with the input (non-drug) material may be substantially similar to that described above with respect to the input (drug material).

FIG. 7O illustrates an Input (Label) Material Window 730O. In one or more examples, the lot quantity field for labels in the lot assignment record is auto calculated based on quantity to be manufactured from the output material multiplied by the bill of materials quantity from the input material label. In one or more examples, there can be multiple label records for the same output material record in the lot assignment record, e.g., a main material and an alternative material. In one or more examples, a user can select which labels to apply to the batch record in the manner described above with respect to FIG. 7M. In one or more example, information from the study batch record related to the input (label) material record cannot be updated.

FIG. 7P illustrates an Edit Example Pack Material Window 730P. In one or more examples, the process for updating and editing information associated with the example pack record may be substantially similar to that described above with respect to the input (drug material). For example, the system can receive additional information related to the lot quantity, supplier lot, supplier lot number, expiry date, and customer lot number. In one or more examples, the system can access the inventory associated with the packaging site to determine the additional information.

FIG. 7Q illustrates an exemplary Common Materials page 720Q according to embodiments of the present disclosure. In one or more examples, the Common Materials page 720Q can include multiple common materials. As shown in the figure, the Common Materials page 720Q can include the navigational user affordance 722, the operational user affordance 724, and the Common Materials information display region 726Q. In one or more examples, the Common Materials information display region 726Q can include, but is not limited to, a plurality of user affordances, information regarding drug input materials (e.g., an item identifier, one or more item descriptions, a UoM, a label status, an alternative material, a bill of materials quantity, a lot quantity, a supplier lot number, an expiry date, and a batch record status), non-drug input materials (e.g., an item identifier, one or more item descriptions, a UoM, a label status, an alternative material, a bill of materials quantity, a lot quantity, a supplier lot number, an expiry date, and a batch record status), label input materials (e.g., an item identifier, one or more item descriptions, a UoM, a label status, a barcode type, a bill of materials quantity, a lot quantity, a supplier lot number, an expiry date, and a batch record status), and example pack materials (e.g., an item identifier, one or more item descriptions, a UoM, a label status, an alternative material, a bill of materials quantity, a lot quantity, a supplier lot number, an expiry date, and a batch record status).

At Block 710 of FIG. 7A, the system can obtain additional information related to the common materials for the lot assignment. In one or more examples, additional information associated with the common materials can be updated as described above with respect Block 708, process 708I, and/or FIGS. 7I-7P.

At Block 712, the system can receive an approval of the study information from the one or more production approvers. In one or more examples, the system may not permit an approval of the lot assignment records to be accessed without receiving completed information regarding the quantity and supplier lot information (e.g., lot quantity, supplier lot number, supplier lot, expiry date, customer lot number). In one or more examples, the process associated with Block 712 can be similar the approval processes described above.

FIG. 7R illustrates an exemplary portion of a Lot Assignment UI 720R that includes an approval user affordance 778R. In one or more examples, the system can receive an indication that the production user has selected the approval user affordance 778R from the operational user affordances. Upon receiving the selection of the approval user affordance 778R, the system can present an approval report 772S. The approval report can include the information associated with the lot assignment UI information display regions, e.g., one or more of Output and Input Materials display region 726Q, Common Material information display region 726Q, Related Batch Records information display region 726U, and Bill of Materials information display region 726V.

Once a user is ready to sign the Approval Report 772S, e.g., after the user has reviewed and verified the information in the Approval Report 772S, the system can receive an indication that the user has selected the signature button 776S. Upon receiving an input corresponding to a selection of the signature button 776S the system can cause a signature window 774T to appear. In one or more examples, the signature window 774T can be overlaid on the Approval Report 772S. The signature window 774T can include one or more user affordances for receiving the credentials of the production user. For example, the signature window 774T can include a textbox for a production user to input their userID and corresponding password. The system can then receive an indication of the production user accepting or rejecting the approval.

In one or more examples, once the lot assignment record is approved, the system may not permit changes to be made to the approved version of the lot assignment record. If a production user desires to make changes to the lot assignment, then the production user can create a new version of the lot assignment. The system can create a new version of the lot assignment by receiving a selection by the user of the New Version button from the operational user affordances 724. Changes to the new version of the lot assignment can be made in accordance with the description provided above, e.g., process 764.

The Lot Assignment UI can further present a list of related batch records. FIG. 7U illustrates an exemplary Related Batch Records (XBRs) page 720U associated with the Lot Assignment UI. In one or more examples, the Related XBRs page 720U can include the plurality of navigational user affordances 722, the plurality of operational user affordances 724, and a Related Batch Records information display region 726U. The Related Batch Records information display region 726U can include a list of existing batch records that are automatically created for each bill of materials upon receiving the approval of the lot assignment.

The Lot Assignment UI can further present a list of related bills of materials. FIG. 7V illustrates an exemplary Bill of Materials page 720V associated with the Lot Assignment UI. In one or more examples, the Bill of Materials page 720V can include the plurality of navigational user affordances 722, the plurality of operational user affordances 724, and a Bill of Materials information display region 726V. The Bill of Materials information display region 726V can include a list of the bill of materials associated with the study batch record that the lot assignment is based upon.

### Batch Record

In one or more examples, the executable batch record can correspond to the packaging and control information obtained in Blocks 152-164. In one or more examples, the executable batch record can refer to a batch record that has been approved by a customer approver. In one or more examples, a batch record may be associated with a corresponding study batch record. In one or more examples, the study instructions specific to the packaging site associated with the batch record, and equipment groups may be imported from the study batch record. In one or more examples, the imported information may not be editable at the batch record stage, e.g., via the Batch Record UI. In one or more examples, a user may be able to add and/or edit new equipment groups, a medication list identification, sample details, and sequence ranges. In one or more examples, an approved executable batch record can be accessed by an operator at a packaging site to package a batch of output materials based on the packaging instructions, bill of materials, and lot assignment, as described above.

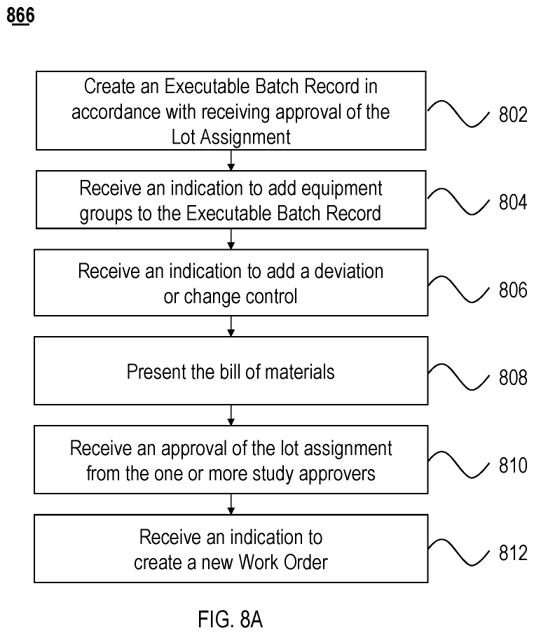

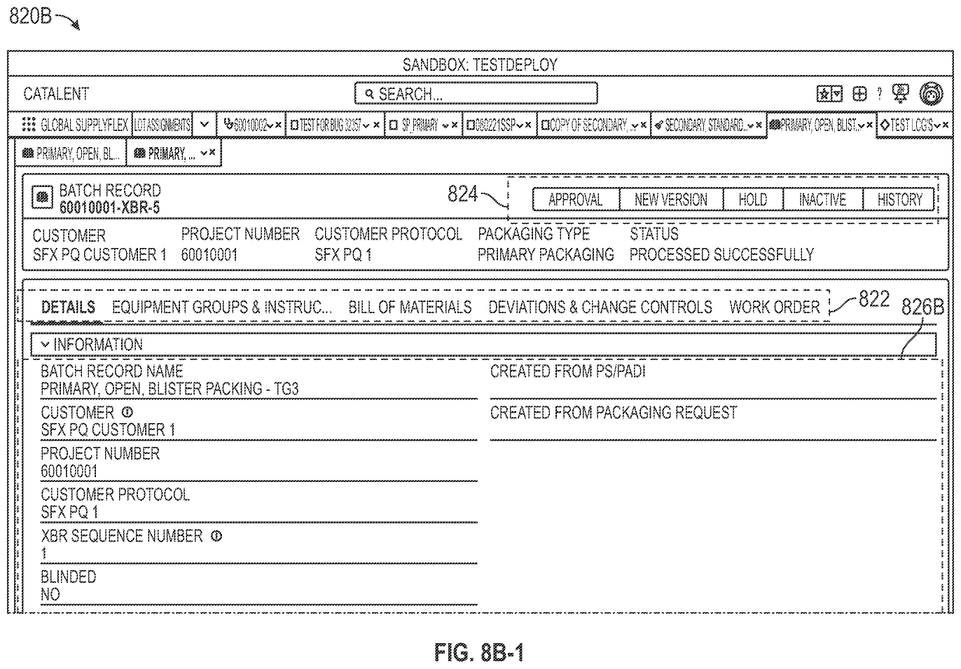

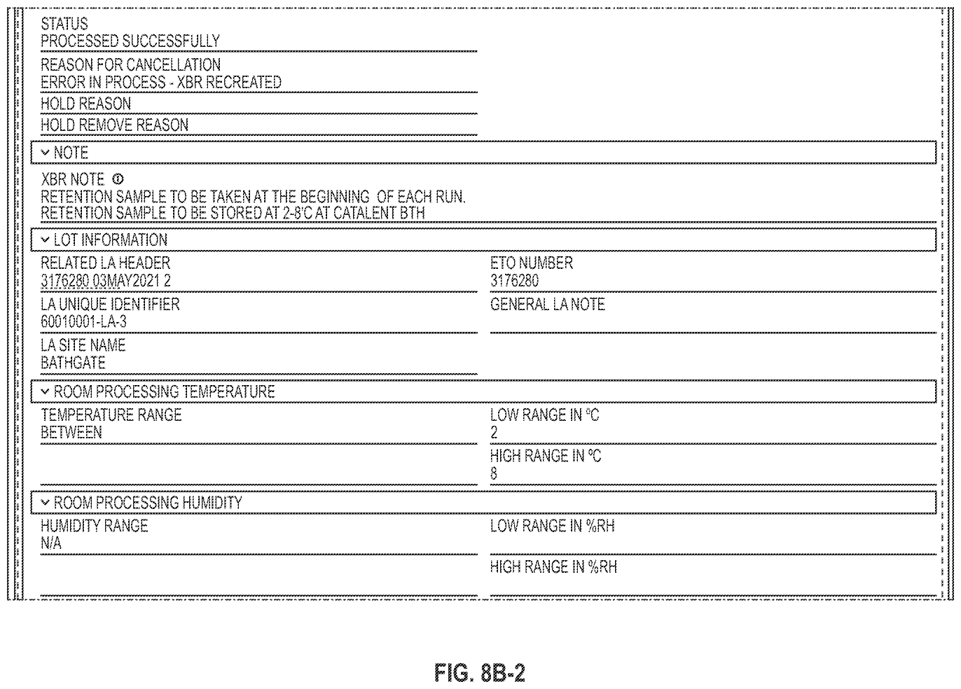

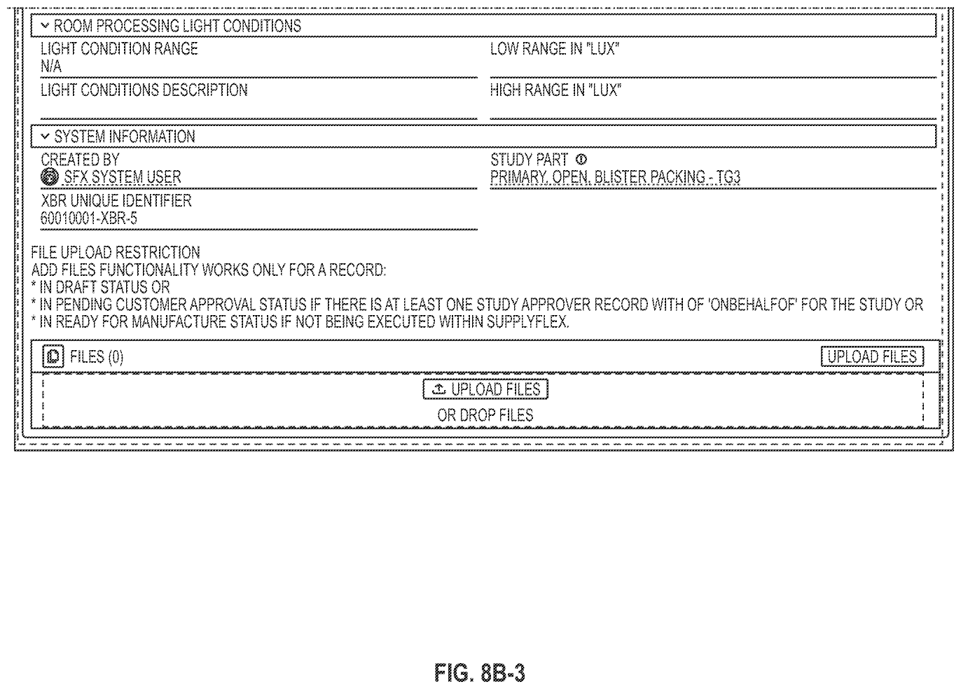

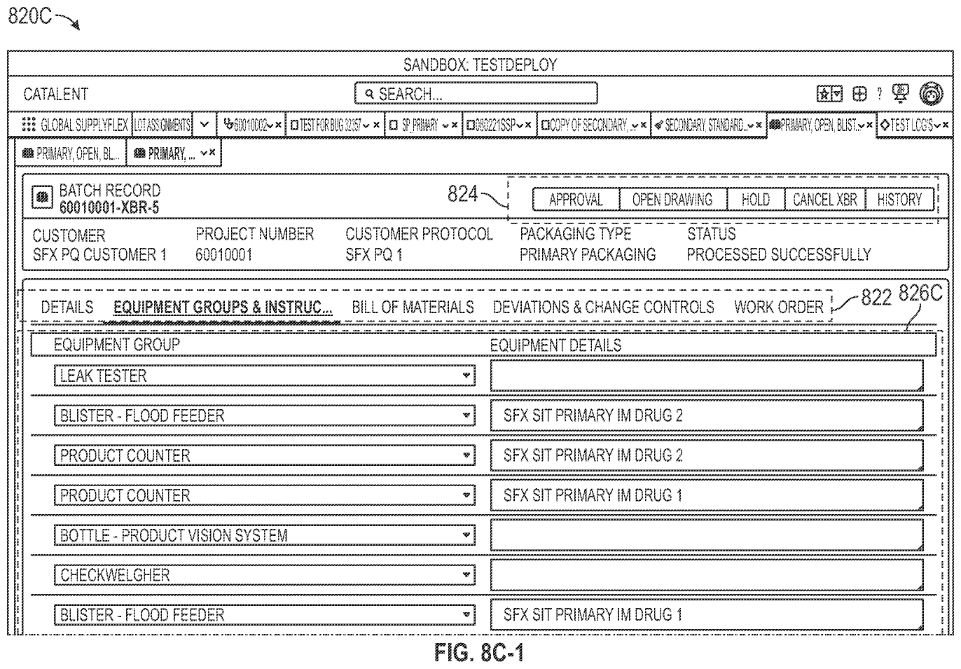

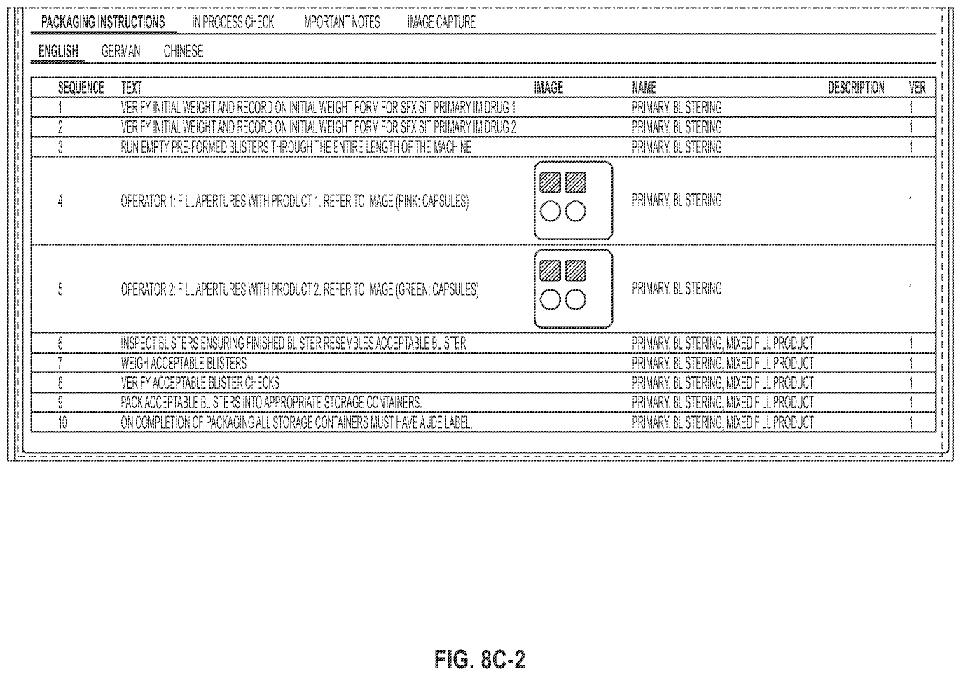

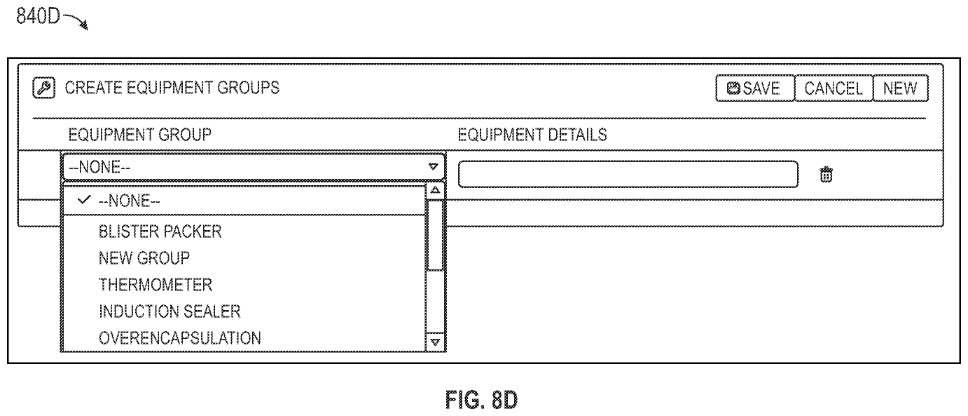

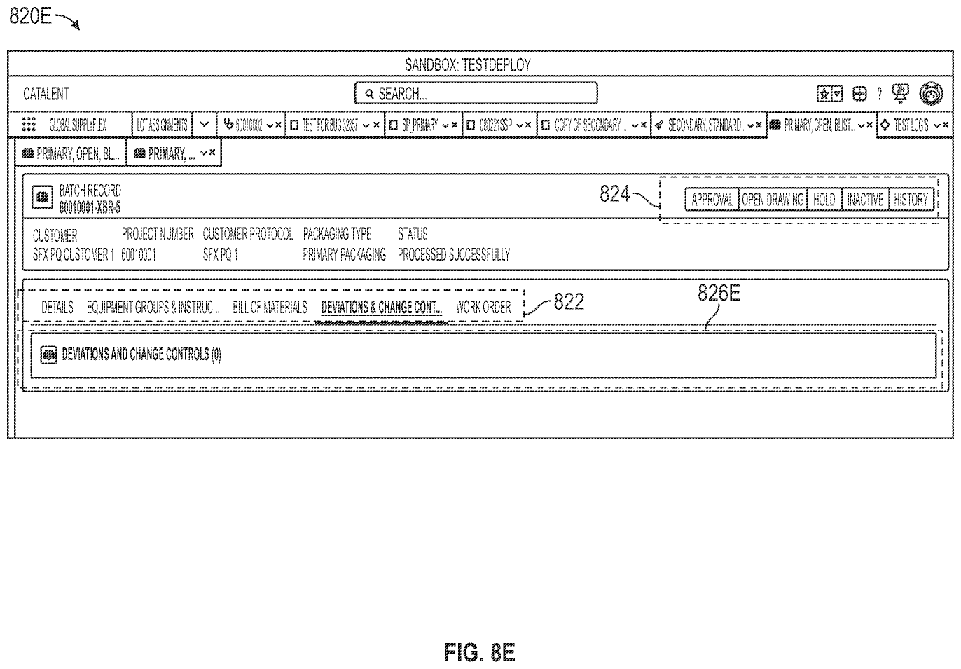

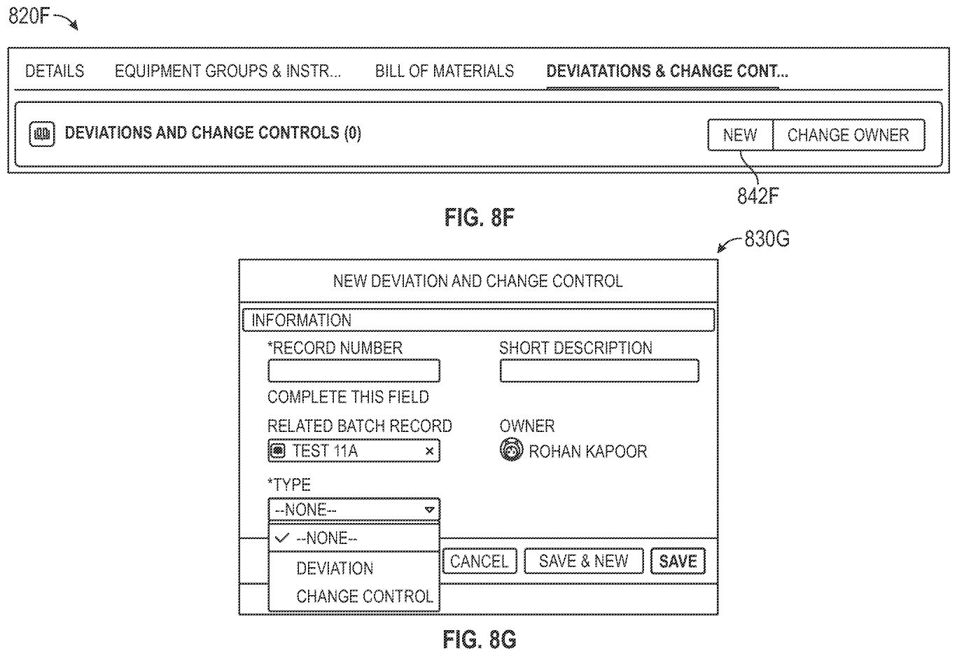

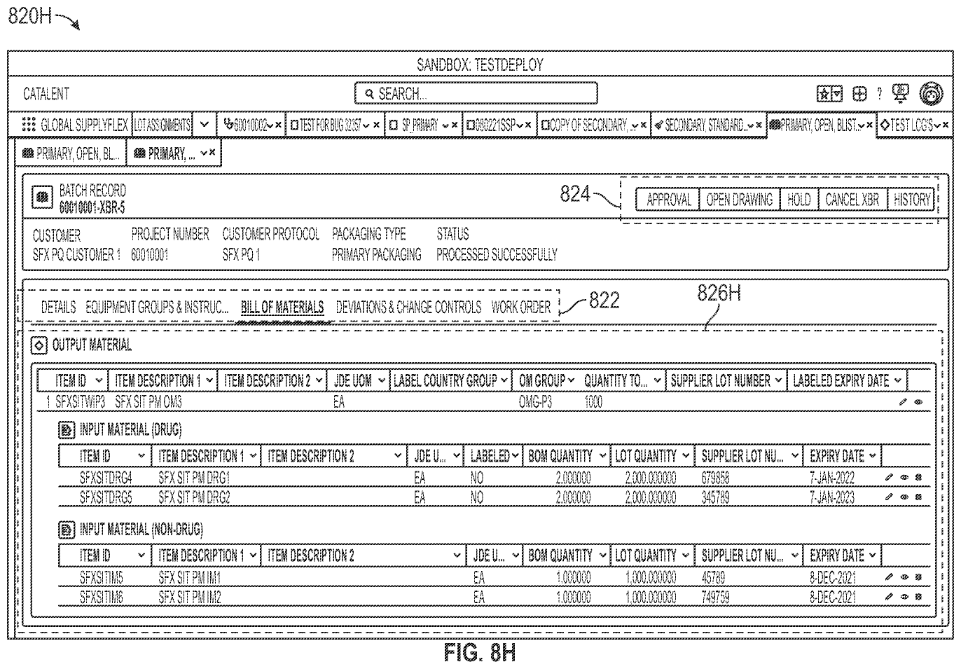

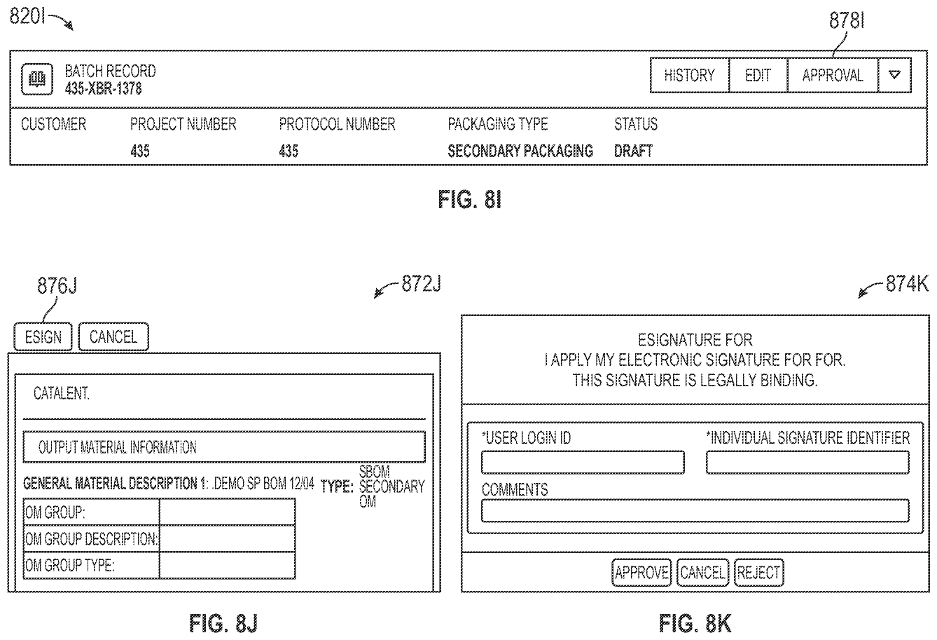

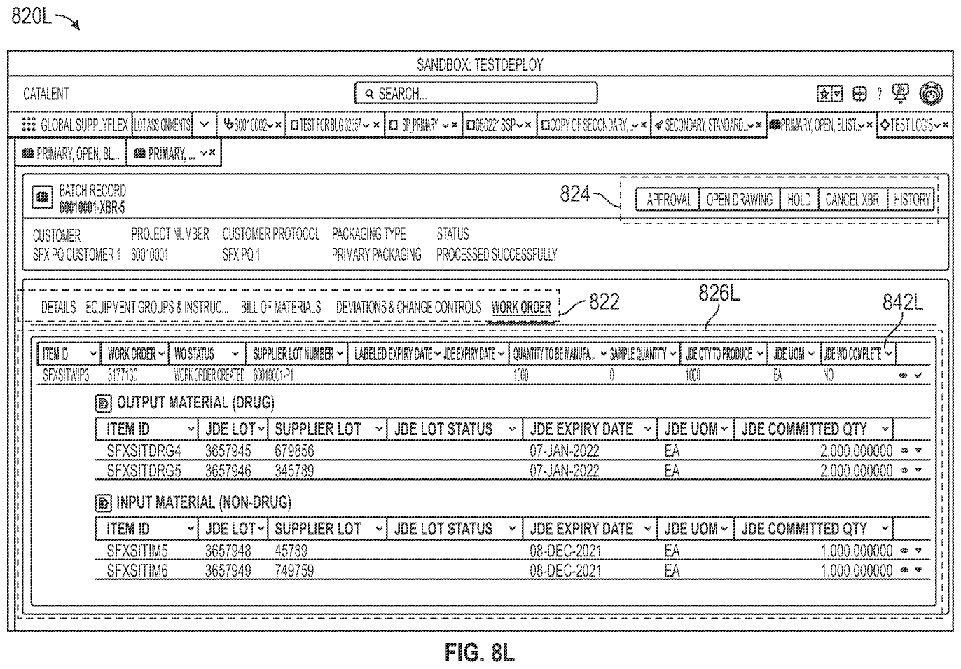

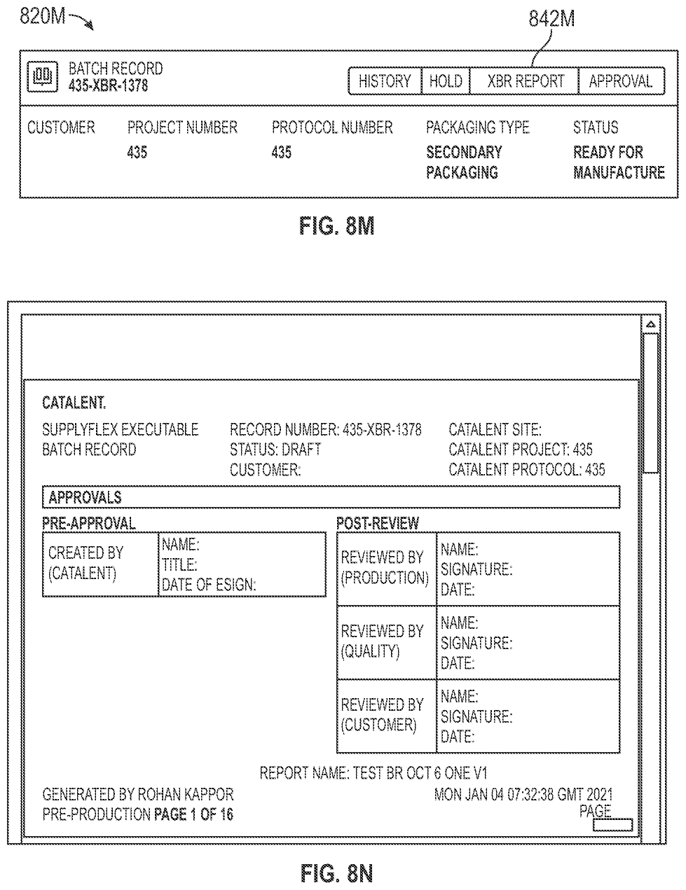

FIG. 8A illustrates a process 866 for approving a batch record, according to one or more embodiments of the present disclosure. In one or more embodiments, process 866 can correspond to Block 166 discussed above with respect to process 150 and process 160. Process 866 can be performed, for example, using one or more electronic devices implementing a software platform. In some examples, process 866 is performed using a client-server system, and the blocks of process 866 are divided up in any manner between the server and a client device. In other examples, the blocks of process 866 are divided up between the server and multiple client devices. In other examples, process 866 is performed using only a client device or only multiple client devices. In process 866, some blocks are, optionally, combined, the order of some blocks is, optionally, changed, and some blocks are, optionally, omitted. In some examples, additional steps may be performed in combination with the process 866. Accordingly, the operations as illustrated (and described in greater detail below) are exemplary by nature and, as such, should not be viewed as limiting.

At Block 802, the system can create a batch record in accordance with receiving approval of the lot assignment. For example, as discussed above, the system can automatically create a batch record upon receiving an approval of the lot assignment (e.g., as described with respect to Block 712).

FIGS. 8B-1, 8B-2, and 8B-3 illustrate an exemplary Batch Record Details page 820B associated with the Batch Record UI, in accordance with embodiments of the present disclosure. As shown in the figure, the Batch Record Details page 820B can include a plurality of navigational user affordances 822, a plurality of operational user affordances 824, and a Batch Record Details information display region 826B. In one or more examples, the Batch Record Details information display region 826B can correspond to an area of the Batch Record UI that displays the information regarding the Batch Record. As shown in the figure, the Batch Record Details information display region 826B can display information included, but not limited to, a batch record name, a customer number, a project number, a customer protocol, whether the materials contain blinding information, a status, a hold reason one or more batch record notes, a related lot assignment header, a lot assignment identifier, a lot assignment site name, a room processing temperature, a room processing humidity, a room processing light conditions, a medication identifier list, a created from PS/PADI indication, a created from packaging request indication, range values, and/or system information. In one or more examples, a user may be able to update one or more fields associated with the Batch Record Details information display region 826B. For example, a user may be able to update a medication identifier list, a created from PS/PADI indication, a created from packaging request indication, range values.

In one or more examples, the Batch Record UI navigational user affordances 822 can include a plurality of user affordances, where each user affordance corresponds to a tab associated with the Batch Record UI and a corresponding page. As shown in the figure, the Batch Record navigational user affordances 822 can include a Batch Record Details tab, an Equipment Groups and Instructions tab, a Bill of Materials Tab, a Deviations and Controls tab, and a Work Order Tab. The examples of navigational user affordances 822 are merely exemplary and more or less navigational user affordances can be used without departing from the scope of this disclosure. A user can navigate between the respective pages associated with the tabs by selecting on the corresponding user affordance.

In one or more examples, the plurality of operational user affordances 824 can include a plurality of buttons that each correspond to a different action that can be implemented by a user. As shown in the figure, the operational user affordances 824 can include, but is not limited to an approval button, an open drawing button, a hold button, a cancel Batch Record button, a history button, and the like. In one or more embodiments, depending on the status of different items associated with the batch record 116, one or more of the operational user affordances 824 may be inactive and/or one or more of the operational user affordances may be removed or made visible. The functions associated with the operational user affordances can be similar to those described above. In one or more examples, a cancel Batch Record button can be used to invalidate an approved batch record. In one or more examples, an open drawing button can be used to open one or more drawings associated with the batch record.

Referring back to FIG. 8A, at Block 804, the system can receive an indication to add one or more equipment groups to the batch record. Adding the equipment group to the batch record can associate an equipment group with the batch record, so that when an approved executable batch record is accessed by an operator at a packaging site, the operator will be able to see what equipment will be necessary to package the batch of output materials. In one or more examples, the system can receive the one or more equipment groups via the Equipment Groups and Packaging Instructions page.

FIGS. 8C-1 and 8C-2 illustrate an exemplary Equipment Groups and Packaging Instructions page 820C associated with the Batch Records UI. The Equipment Groups and Packaging Instructions page 820C can include a plurality of navigational user affordances 822, a plurality of operational user affordances 824, and an Equipment Groups and Packaging Instructions information display region 826C. In one or more examples, the Equipment Groups and Packaging Instructions information display region 826C can correspond to an area of Batch Records UI that displays the information regarding the equipment groups and packaging instructions. As shown in the figure, the Output and Input Materials information display region 826C can be similar to the respective information display regions associated with the Instructions and Equipment (Study Instructions-Packaging Instructions) page 520E and Instructions and Equipment (Equipment Groups) page 520L. In one or more examples, a user can navigate to pages within the Equipment Groups and Packaging Instructions page that may be substantially similar to the Instructions and Equipment (Study Instructions—In Process Check) page 520F and Instructions and Equipment (Study Instructions-Important Notes) page 520G, as described above.

FIG. 8D illustrates how a user can add an equipment group to a batch record. FIG. 8D shows a portion of an Equipment Groups and Packaging Instructions page 820D that can receive an indication of an additional equipment group to be added to the batch record. As shown in the figure, the system can receive a new equipment group via a drop down menu and/or a text input regarding equipment details.

At Block 806, the system can receive an indication to add a deviation or change control. The deviation and/or change controls can include reasons for deviating from the packaging instructions and what steps should be taken. This can provide operators at the packaging site an explanation as to why they may need to deviate from the packaging instruction and how to do so. For example, a deviation may occur if it is discovered that the seals on the bottles are faulty. The deviations included in the batch record can provide an explanation as to why the operators should de-bottle the batch.

FIG. 8E illustrates an exemplary Deviations and Change Controls page 820E associated with the Batch Records UI. The Deviations and Change Controls page 820E can include a plurality of navigational user affordances 822, a plurality of operational user affordances 824, and a Deviations and Change Controls information display region 826E. In one or more examples, the Deviations and Change Controls information display region 826E can include a list of deviations and change controls for a batch record.

FIG. 8F illustrates a portion of an exemplary Deviations and Change Controls page 820F associated with the Batch Records UI. The system can receive an indication to add a deviation or change control when a user selects new-deviation user affordance 842F. Upon receiving a selection of the new-deviation user affordance 842F, the system can navigate to New Deviation and Change Control Window 830G, as shown in FIG. 8G. The New Deviation and Change Control Window 830G can include a plurality of input user affordances for a user to enter details regarding the deviation and/or change control. As shown in the figure, the user affordances can include text entry boxes, drop down menus, and the like. In one or more examples, the deviation and/or change control details can include but is not limited to a record number, a short description, a related batch record and a type.

At Block 808, the system can present the bill of materials to a user. For example, the system can be navigated to the Bill of Materials page associated with the Batch Record UI. In one or more examples, the user may review the bill of materials prior to approving the batch record.

FIG. 8H illustrates an exemplary Bill of Materials page 820H. The Bill of Materials page 820H can include a plurality of navigational user affordances 822, a plurality of operational user affordances 824, and an Bill of Materials information display region 826H. In one or more examples, the Deviations and Bill of Materials information display region 826H can include the output materials and corresponding input materials associated with the batch record.

At Block 810, the system can receive an approval of the batch record from the one or more production users, e.g., production approvers. In one or more examples, the process associated with Block 810 can be similar the approval processes described above for approval by a production user.

FIG. 8I illustrates an exemplary portion of a batch record UI 8201 that includes an approval user affordance 878I. In one or more examples, the system can receive an indication that the user has selected the approval user affordance 878I from the operational user affordances. Upon receiving the selection of the approval user affordance 878I, the system can present an approval report 872J. The approval report can include the information associated with the Batch Record UI information display regions, e.g., one or more of the Bill of Materials information display region 826H, Equipment Groups and Instructions information display region 826C, Deviations and Change Controls information display region 826E.

Once a production user is ready to sign the Approval Report 872J, e.g., after the production user has reviewed and verified the information in the Approval Report 872J, the system can receive an indication that the production user has selected the signature button 876J. Upon receiving an input corresponding to the signature button the system can cause a signature window 874K to appear, as shown in FIG. 8K. In one or more examples, the signature window can be overlaid on the Approval Report 872J. The signature window 874K can include one or more user affordances for receiving the credentials of the production user. For example, the signature window 874K can include a textbox for a production user to input their userID and corresponding password. The system can then receive an indication of the production user accepting or rejecting the approval.

At Block 812, the system can receive an indication to create a new Work Order. In one or more examples, once the batch record is approved, a new navigational user affordance 822, (e.g., “Work Order” navigational user affordance) may be visible in the Batch Record UI. In one or more examples, if the status of the work order is “Work Order Created,” a production user can manually the status of the Work order to complete.

FIG. 8L illustrates an exemplary Work Order page 820L associated with the Batch Records UI. The Work Order page 820L can include a plurality of navigational user affordances 822, a plurality of operational user affordances 824, and an Work Order information display region 826L. In one or more examples, Work Order information display region 826L can correspond to an area of the Batch Records UI that displays the information regarding the work order, including but not limited to an item identifier, a work order number, a work order status, a supplier lot number, one or more expiry dates, a quantity to be manufactured, a sample quantity, a quantity to produce, a UoM, a Work Order status, and information associated with the input (drug) materials (e.g., customer lot, supplier lot, customer lot status, one or more expiry dates, UoM, and customer committed quantity), and input (non-drug) materials (e.g., customer lot, supplier lot, customer lot status, one or more expiry dates, UoM, and customer committed quantity). In one or more examples, a user can use the WO status field 842L to manually update the status of the work order.

In one or more examples, once all work orders associated with a lot assignment are complete and/or approved by a customer approver, the status of the batch record may change to “ready for manufacture.” Once the executable batch record is associated with a “ready to manufacture” status, the executable batch record, e.g., an executable batch record report, can be accessed and viewed by clicking the “XBR Report” user affordance 842M, as shown in FIG. 8M. In one or more examples, the executable batch record report can be printed and used by an operator at a packaging site to package the output materials described in the executable batch record. FIG. 8N shows an exemplary page of an executable batch record report according to embodiments of this disclosure.

### Customer User Interface

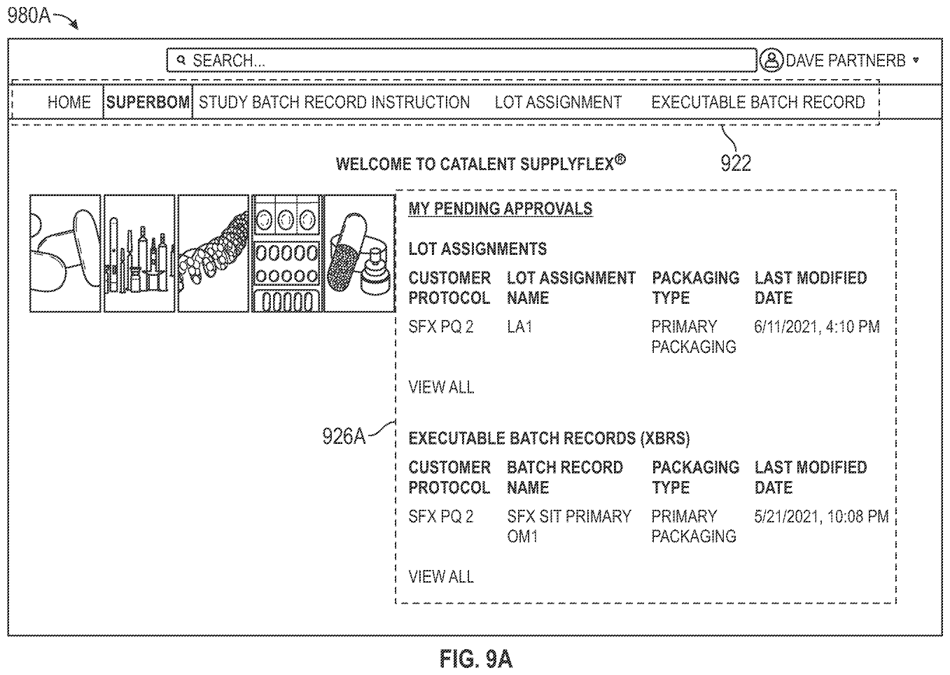

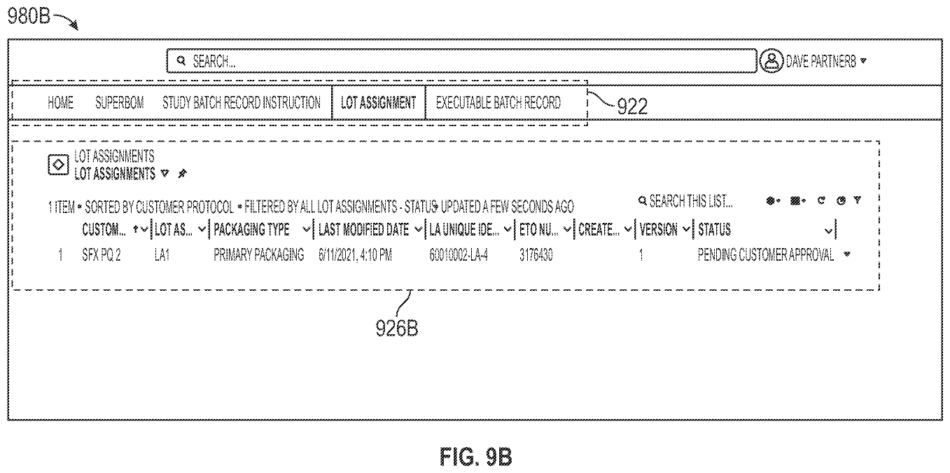

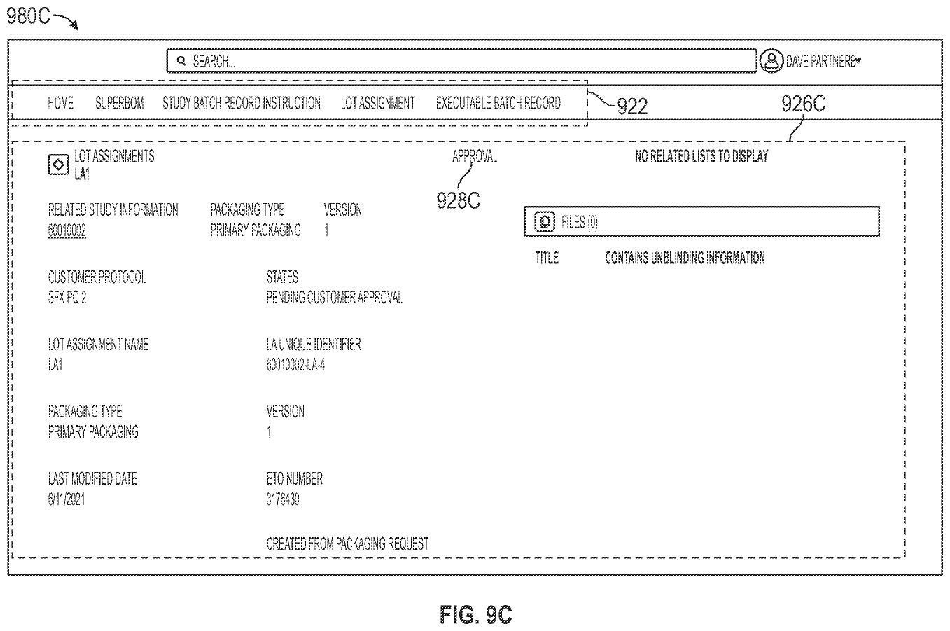

Customer User Interface, e.g., customer UI 180, provides a portal or user interface where the customer and/or individuals associated with the customer can review study components (e.g., bill of materials, study instructions, lot assignment, and batch records) associated with one or more studies. FIG. 9A illustrates an exemplary Customer UI Home page 980A. The Customer UI Home page 980A may be unique to a particular customer user. For example, the Customer UI Home page 980A may be presented to customer user A upon login to the system. The pending approvals displayed for the customer user A may be based on the information entered at Block 154 designated customer study approvers. Accordingly, customer user B may have a different Customer UI Home page corresponding to pending approvals for customer user B.

As shown in the figure, the Customer UI Home page 980A can include a navigation bar 922 and a list of pending approvals 926A. If there are no pending approvals associated with the customer user, then the list of pending approvals 926A may be hidden and/or empty. As shown in the figure, the navigation bar 922 may include a plurality of user affordances for the customer user to navigate between different pages. As shown in the figure, the navigation bar includes a Home tab, a Bill of Materials tab, a Study Instructions tab, a Lot Assignment Tab, and a Batch Record tab. The system can navigate to a page corresponding to a tab selected by a customer user. Each of the corresponding pages can include a list of items associated with the respective study component. For example, the Bill of Materials page may include a list of Bills of Materials associated with the customer user that are pending approval or have received approval. The Study Instructions page may include a list of Study Instructions associated with the customer user that are pending approval or have received approval. The Lot Assignment page may include a list of Lot Assignments associated with the customer user that are pending approval or have received approval. The Batch Record page may include a list of Batch Records associated with the customer user that are pending approval or have received approval.

FIG. 9B illustrates an exemplary Lot Assignment page 980B. The Lot Assignment page 980B includes a list of Lot Assignments 926B associated with the customer user that are pending approval or have received approval. In one or more examples, each list item (e.g., each lot assignment in the list) can include a link, that when selected, will cause the system to present a summary of the corresponding item (e.g., display the lot assignment).

FIG. 9C illustrates an exemplary Lot Assignment Summary page 980C, according to embodiments of the present disclosure. In one or more examples, the Lot Assignment Summary page 980C can include information associated with the Lot Assignment including, but not limited to, a version, a packaging type, a customer protocol, a status, a lot assignment name, a lot assignment identifier, an ETO number, and associated files including a Lot Assignment Approval Report.

The Lot Assignment Summary page 980C can further include an Approval user affordance 982C. Upon receiving a selection of the Approval user affordance 982C, the system can present an approval window as described above with respect to approvals for production users. In one or more examples, the customer approval window may include a user affordance for the customer user to provide their credentials and/or their signature a signature. Upon receiving the customer approval, the system can update the corresponding information (e.g., lot assignment 114). This approval process may correspond to the Block 186 described above.

A skilled artisan will understand that the Bill of Materials page, Study Instructions page, and Batch record page may be similar to the Lot Assignment page 980B and Lot Assignment Detail page 980C. Accordingly, approval for the Bill of Materials page, Study Instructions page, and Batch record page may be similar to the approval process for the Lot Assignment.

### Auditing

In clinical trial studies, audits may occasionally be conducted to evaluate the clinical trial conduct and compliance with various regulatory requirements. Accordingly, the system is configured to generate an audit history with respect to actions taken within the system from the creation of a study information to the approval of the executable batch record. In one or more instances, every action that is completed in the system with respect to each of the study components, e.g., the master data 102, study information 104, study part 106, study batch record 108, study instructions 110, bill of materials, 112, lot assignment 114, and executed batch record 116, and various approvals can be included in the audit history. In one or more examples, the audit history can include a list of actions and the corresponding user that completed the action with respect to each of the study components.

In one or more examples, if a study is being audited, a production user may log on to the system and retrieve the auditing history associated with the study. In one or more examples, the production user can print the auditing history to be sent to the agency conducting the audit. In one or more examples, the auditing process shown and described with respect to this system can comply with the current governmental regulations.

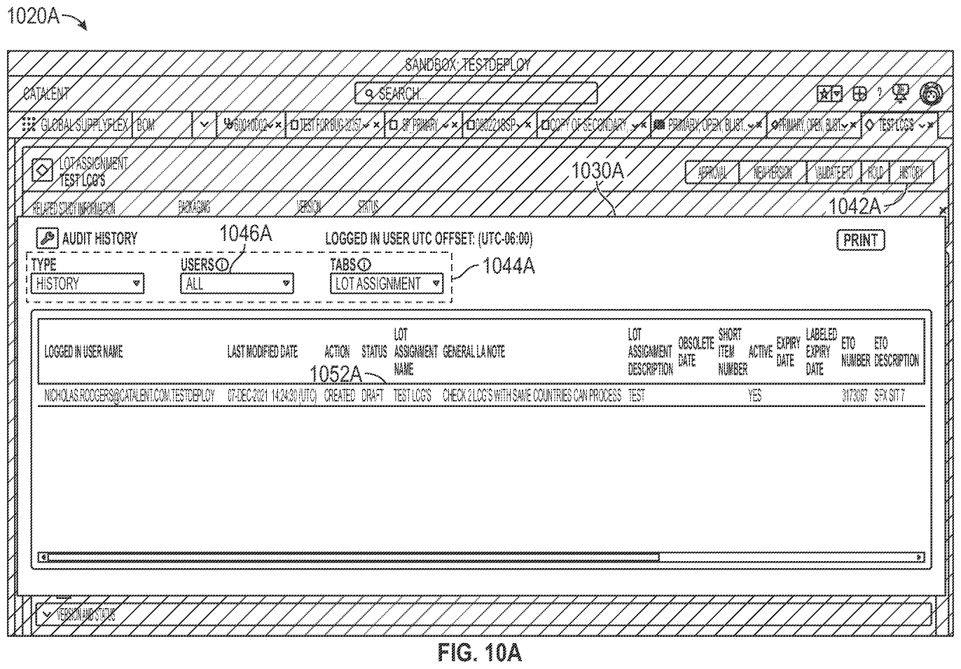

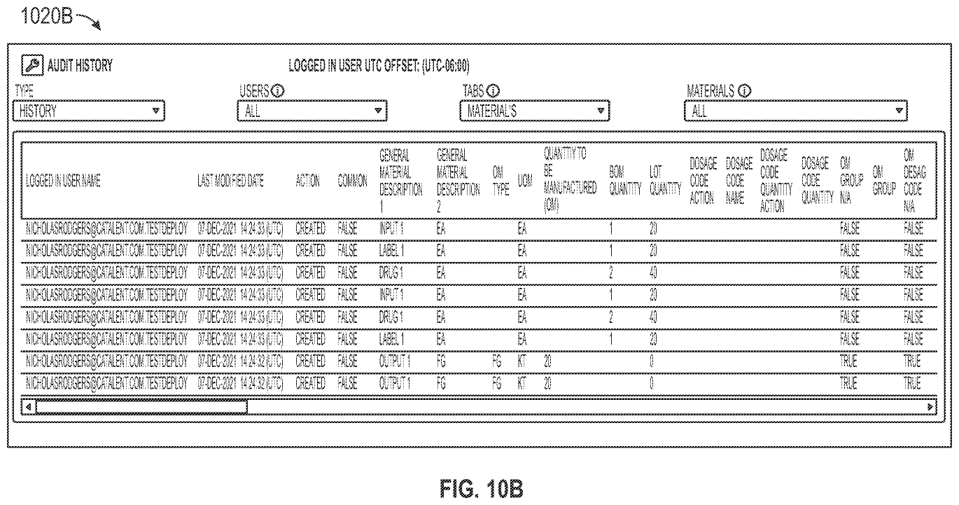

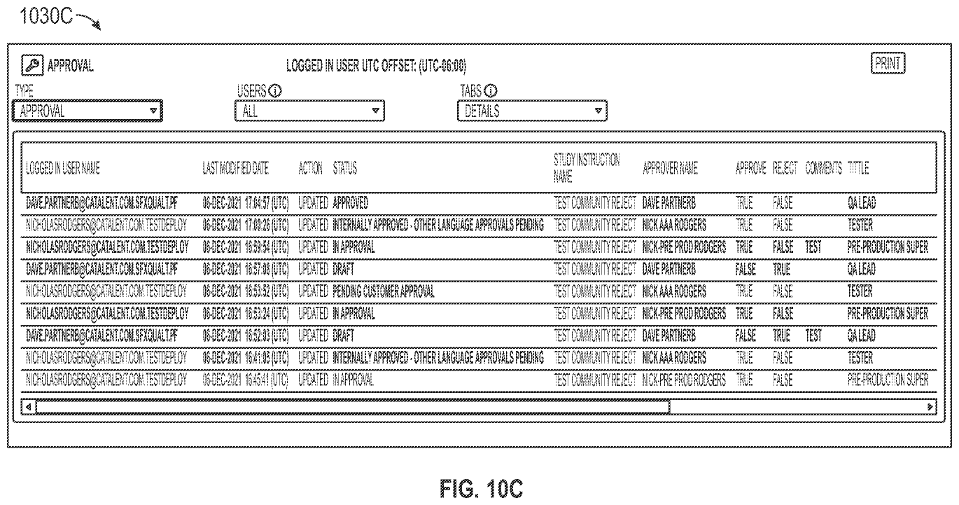

FIG. 10A illustrates an exemplary Lot Assignment Details page 1020A, according to embodiments of the present disclosure. As shown in the figure, the Lot Assignment Details page 1020A can include a “History” user affordance 1042A. In one or more examples, when the system receives an indication that a production user has selected the History user affordance 1042A, the system can present a corresponding Audit History Window 1030A, that includes a list of Lot Assignments. While the figure illustrates a single entry, a skilled artisan will understand that one or more entries may be included in the audit history window 1030A, depending on the selected fields 1044A.

A production user can select a link corresponding to an entry in the list. For example, based on Audit History Window 1030A, the system can receive an indication that the user selected “Test LCG's” Lot Assignment 1052A. Upon receiving a selection of an entry in the list, e.g., “Test LCG's” Lot Assignment 1052A, the system can present the audit history for the selected entry. For example, FIG. 10B illustrates an exemplary audit history 1020B for the “Test LCG's” Lot Assignment. As shown in the figure, the audit history can include, but is not limited to, an action, a corresponding user, a date of the action, and information associated with the particular study component (e.g., for the lot assignment, this information can include one or more general material descriptions, an output matter type, UOM, a quantity to be manufactured, a bill of materials quantity, a lot quantity, a dosage code action, a dosage code quantity action, a dosage code quality, an output material group, and output material dosage code, and the like. A skilled artisan will understand that the information associated with the particular study component (e.g., study information, study part, study batch record, study instructions, bill of materials, lot assignment, batch record) may vary based on which study component is being audited.

FIG. 10C illustrates another exemplary window that may be accessed for auditing purposes. As shown in the figure Approval Flow Window 1030C can include a history of the records that were included in the study component as part of an approval flow (i.e., from the point of time, when a record is submitted for approval till the record receives an approval or rejection) of the record.

In one or more examples, the system can generate a user specific audit report by selecting a particular user (e.g., customer user or production user). For example, with reference to Audit History Window 1030A, a specific user can be selected from the “Users” user affordance 1046A. Upon selecting the “Users” user affordance 1046A, the system can display the changes to the relevant study component specific to the selected user.

### Blinding Studies

The system can be configured to preserve blinding information when presenting the completed study components to a customer user for approval. As discussed above, a production facilitator can indicate whether the study will be blinded. In such examples, information associated with the study may be redacted and/or unavailable to customer users that are indicated as blinded in the system. In this manner, the system can implement a blinding protocol to redact unblinding information to ensure that it is not shared with the customer.

In contrast, other processes for blinding a study rely on manual human labor and may be prone to errors. For example, when preparing a batch record for a customer, a production facilitator may manually redact the batch record prior to sending to the customer. In one or more examples, an individual associated with the production facilitator may have to replace a page of the batch record with a redacted page to redact information associated with kit numbers and sequence numbers listed on the batch record. Not only does this process rely on manual human labor, but it is not particularly secure, e.g., the production facilitator may neglect to send unblinding information, which would then unblind the study.

Embodiments according to the present disclosure, however, provide a secure process to blind the study without relying on significant manual human labor. For example, as discussed above, a production user inputting the study information can indicate whether a study is blinded and can designate which customer user, including customer approvers, are blinded. Once the study and customer users are indicated as blinded, the system will automatically redact and/or withhold unblinding information from the blinded customer users. In this manner, embodiments according to the present disclosure provide a secure method to maintain a blinded study throughout the process of generating an executable batch record.

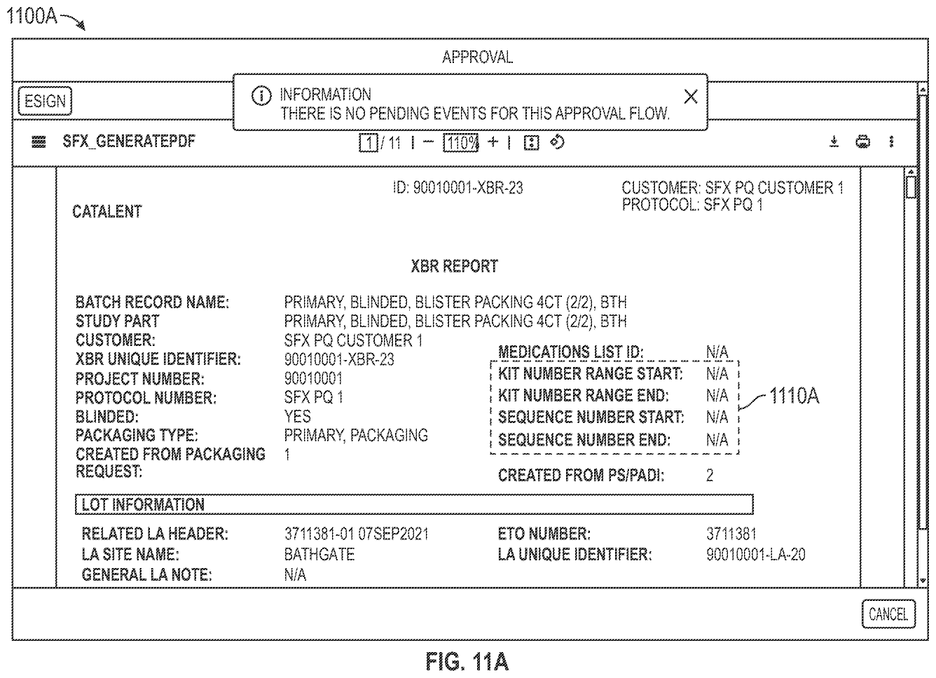

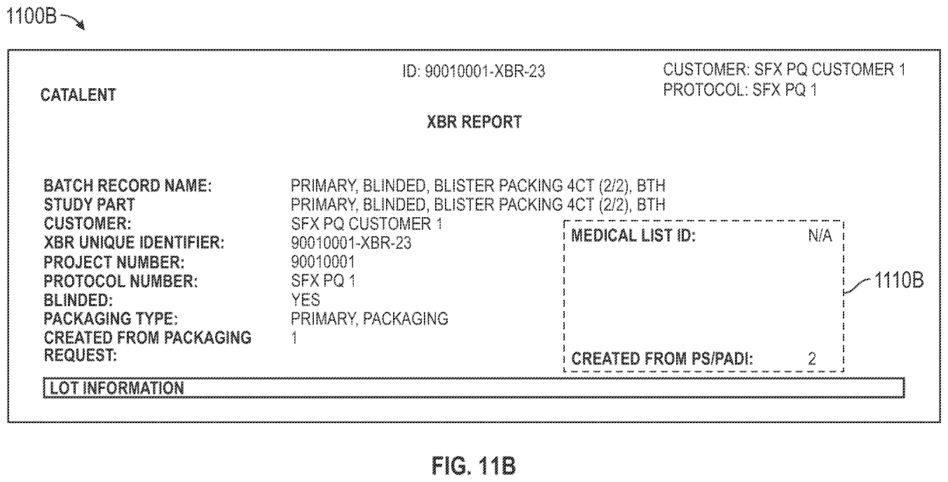

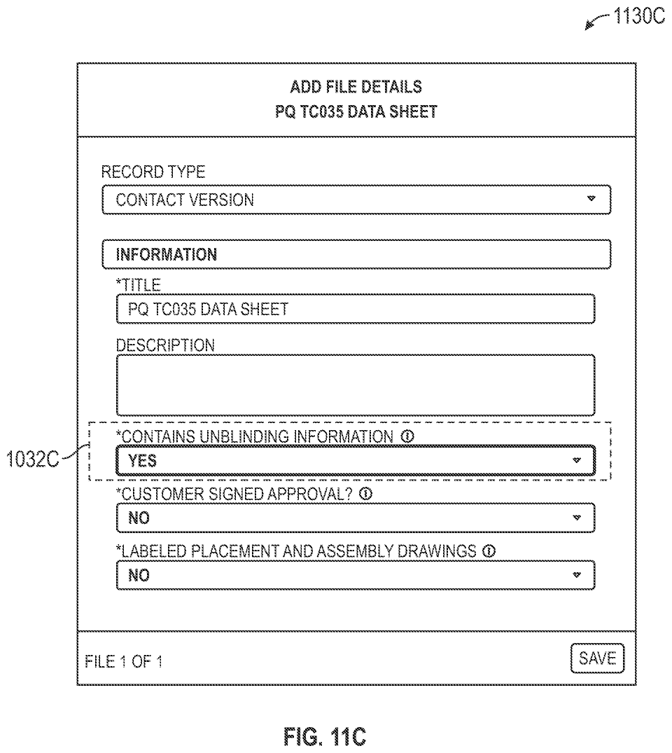

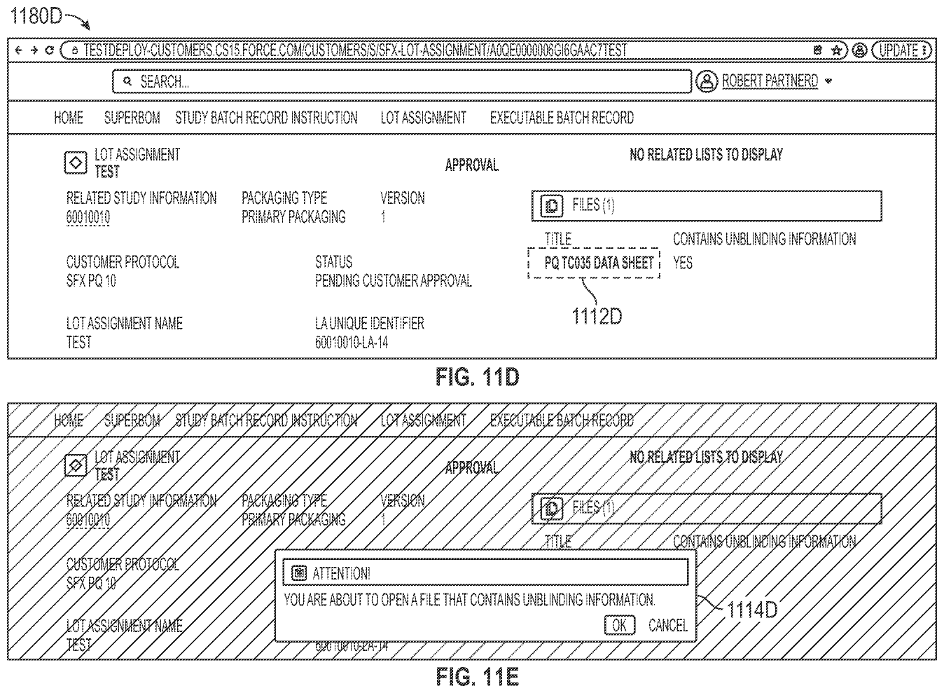

In one or more examples, unblinding data associated with a batch record can be redacted for blinded customer users. FIG. 11A shows a view of a batch record report 1100A for a blinded study presented to an unblinded customer user. As shown in the figure the batch record report 1100A displays unblinding information 1110A, including but not limited to the kit number range and the sequence number range. FIG. 11B shows a view of n batch record report 1100B for a blinded study presented to a blinded customer user. As shown in the figure the batch record report 1100B redacts (e.g., does not display) unblinding information in region 1110B. In this manner, the system can automatically redact the unblinding information for customer users who are designated as blinded.

Additionally, to the extent that files uploaded to the study instructions 110, bill of materials 112, lot assignment 114, and batch record 116 include unblinding information, these files may be unavailable for viewing by a blinded customer user. FIG. 11C shows an exemplary File Upload Window 1130C according to embodiments of the present disclosure. As shown in the figure, the File Upload Window 1130C can include a user affordance 1032C to indicate whether the file includes unblinding information.

Upon receiving an indication that a file includes unblinding information, the system can mark the file as containing unblinding information. For example, FIG. 1180D illustrates an exemplary Customer Lot Assignment Detail page 1180D presented via the Customer UI 180 that includes a file marked as containing unblinding information. In one or more examples, the system can deactivate file link 1112D. In one or more examples, the file link 1112D can be active for unblinded customers. Upon receiving a selection of the file link 1112D from an unblinded customer user, the system can display a warning window 1114E, informing the unblinded customer user that they are about to view a document containing unblinding information. The system can receive a selection from the unblinded customer user to open and present the file or to not open the file and return to the details page.

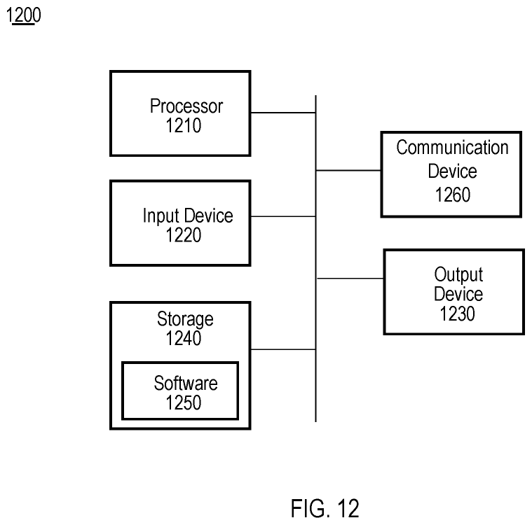

The operations described above with reference to the above-described figures are optionally implemented by components depicted in FIG. 12. It would be clear to a person having ordinary skill in the art how other processes are implemented based on the components depicted in FIG. 12.

FIG. 12 illustrates an example of a computing device in accordance with one embodiment. Device 1200 can be a host computer connected to a network. Device 1200 can be a client computer or a server. As shown in FIG. 12, device 1200 can be any suitable type of microprocessor-based device, such as a personal computer, workstation, server or handheld computing device (portable electronic device) such as a phone or tablet. The device can include, for example, one or more of processor 1210, input device 1220, output device 1230, storage 1240, and communication device 1260. Input device 1220 and output device 1230 can generally correspond to those described above, and can be either connectable or integrated with the computer.

Input device 1220 can be any suitable device that provides input, such as a touch screen, keyboard or keypad, mouse, or voice-recognition device. Output device 1230 can be any suitable device that provides output, such as a touch screen, haptics device, or speaker.

Storage 1240 can be any suitable device that provides storage, such as an electrical, magnetic or optical memory including a RAM, cache, hard drive, or removable storage disk. Communication device 1260 can include any suitable device capable of transmitting and receiving signals over a network, such as a network interface chip or device. The components of the computer can be connected in any suitable manner, such as via a physical bus or wirelessly.

Software 1250, which can be stored in storage 1240 and executed by processor 1210, can include, for example, the programming that embodies the functionality of the present disclosure (e.g., as embodied in the devices as described above).

Software 1250 can also be stored and/or transported within any non-transitory computer-readable storage medium for use by or in connection with an instruction execution system, apparatus, or device, such as those described above, that can fetch instructions associated with the software from the instruction execution system, apparatus, or device and execute the instructions. In the context of this disclosure, a computer-readable storage medium can be any medium, such as storage 1240, that can contain or store programming for use by or in connection with an instruction execution system, apparatus, or device.

Software 1250 can also be propagated within any transport medium for use by or in connection with an instruction execution system, apparatus, or device, such as those described above, that can fetch instructions associated with the software from the instruction execution system, apparatus, or device and execute the instructions. In the context of this disclosure, a transport medium can be any medium that can communicate, propagate or transport programming for use by or in connection with an instruction execution system, apparatus, or device. The transport readable medium can include, but is not limited to, an electronic, magnetic, optical, electromagnetic or infrared wired or wireless propagation medium.

Device 1200 may be connected to a network, which can be any suitable type of interconnected communication system. The network can implement any suitable communications protocol and can be secured by any suitable security protocol. The network can comprise network links of any suitable arrangement that can implement the transmission and reception of network signals, such as wireless network connections, T1 or T3 lines, cable networks, DSL, or telephone lines.

Device 1200 can implement any operating system suitable for operating on the network. Software 1250 can be written in any suitable programming language, such as C, C++, Java or Python. In various embodiments, application software embodying the functionality of the present disclosure can be deployed in different configurations, such as in a client/server arrangement or through a Web browser as a Web-based application or Web service, for example.

Embodiments of the present disclosure include systems and methods for generating an executable batch record for a clinical trial study. For example, in one or more embodiments, the method can include, at a first electronic device associated with a production facilitator, displaying a user interface (UI) for inputting batch record information, the UI including at least packaging instructions, a bill of materials, and a lot assignment. In one or more examples, the batch record information can include a selection of inventory associated with a production site and a selection of inventory associated with a pharmaceutical customer. In one or more embodiments, the method can further include, at the first electronic device, receiving the packaging instructions, the bill of materials, and the lot assignment from a user associated with the production facilitator. In one or more embodiments, the method can further include, producing a preliminary batch record based on the information input by the user associated with the production facilitator. In one or more embodiments, the method can further include, at a second electronic device associated with a pharmaceutical batch customer, displaying a customer user interface that comprises the preliminary batch record and an interface for approving the preliminary batch record. In one or more embodiments, the method can further include, at the second electronic device, receiving an approval of the preliminary batch record from a user associated with the pharmaceutical batch customer. In one or more embodiments, the method can further include, in accordance with receiving the approval of the preliminary batch record producing an executable batch record for a producer to execute.

In one or more examples, according to methods of the present disclosure, the user associated with the pharmaceutical batch customer can be configured to approve the preliminary batch record without editing the preliminary batch record. In one or more examples, according to methods of the present disclosure, receiving the packaging instructions can include receiving packaging instructions in a first language, determining whether the first language is used at the production site, and in accordance with a determination that the first language is not used at the production site, generating a translation of the packaging instructions corresponding to a second language used at the production site.

In one or more examples, according to methods of the present disclosure, receiving the lot assignment can include receiving a quantity of output materials to be manufactured, each output material corresponding to one or more input materials, accessing an inventory of a production site, for each output material, assigning a first supplier lot number based on at least one of a first supplier lot size or a first supplier lot expiration date determined from the inventory, and for each input material, assigning a second supplier lot number based on at least one of a second supplier lot size or a second supplier lot expiration date determined from the inventory. In one or more examples, the method can further include generating a second executable batch record for the clinical trial study, including receiving, at the first electronic device, a second lot assignment associated with the production site. In one or more examples, receiving the second lot assignment can include receiving a second quantity of output materials to be manufactured, each output material corresponding to one or more input materials, accessing the inventory of the production site, for each output material, assigning a third supplier lot number based on at least one of a third supplier lot size or a third supplier lot expiration date determined from the inventory of the production site, and for each input material, assigning a fourth supplier lot number based on at least one of a fourth supplier lot size or a fourth supplier lot expiration date determined from the inventory of the production site. In one or more examples, the method can further include producing a second preliminary batch record that is based on the information input by the user associated with the production facilitator; receiving, from the second electronic device, an approval of the second preliminary batch record; and in accordance with receiving the approval of the second preliminary batch record producing a second executable batch record for the producer to execute.

In one or more examples, methods according to the present disclosure can include, receiving at the first electronic device, customer data associated with the pharmaceutical batch customer, study information for the clinical trial study, the clinical trial study associated with the customer data, and output material information, the output material information corresponding to one or more output materials associated for the clinical trial study. In one or more examples, receiving the bill of materials can further include importing at least a portion of the output material information to the bill of materials, receiving a quantity associated with each of the one or more output materials and corresponding input materials from the output material information, and assigning a customer identifier based on the customer inventory. In one or more examples, the study information can include packaging sites, study languages, destination countries, output materials, dosage codes, business units, approvers, or a combination thereof. In one or more examples, the output material information can include output materials, input materials, drug categorizations, alternative materials, storage temperature, time out environment details, humidity control details, light control details, label details or a combination thereof.

In one or more examples according to the present disclosure can include, the executable batch record can be accessed by the user associated with the production facilitator prior to being accessed by the producer. In one or more examples, methods according to the present disclosure can include causing the first electronic device to present an audit history, the audit history corresponding to a list of actions associated with inputting the batch record information and receiving the approval of the preliminary batch record.

In one or more examples, methods according to the present disclosure can include, receiving, at the first electronic device, study information for the clinical trial study, the study information comprising one or more customer approvers, the one or more customer approvers comprising the user associated with the pharmaceutical batch customer. The methods can further include determining whether the batch record information includes blinding information and in accordance with a determination that the batch record information comprises blinding information, causing to present, at the second electronic device, a restricted view of the preliminary batch record that omits the blinding information to the user associated with the pharmaceutical batch customer. In such examples, the user associated with the pharmaceutical batch customer can be configured to be blinded. In such examples, the blinding information can include a medication list, an identification number, a kit number, a start sequence number, an end sequence number, or a combination thereof. In such examples, the one or more customer approvers can further include a second user associated with the pharmaceutical batch customer, wherein the second user associated with the pharmaceutical batch customer is configured to not be blinded. In such examples, methods according to the present disclosure can further include, in accordance with the determination that the batch record information comprises blinding information, causing to present to the second user associated with the pharmaceutical batch customer, an unrestricted view of the executable batch record that includes the blinding information.

Embodiments of the present disclosure can further include an electronic system for generating an executable batch record for a clinical trial study. The electronic system can include one or more processors, a memory, and one or more programs, the one or more programs stored in the memory and configured to be executed by the one or more processors. According to embodiments of the present disclosure, the one or more programs can include instructions for at a first electronic device associated with a production facilitator, displaying a user interface (UI) for inputting batch record information, the UI including at least packaging instructions, a bill of materials, and a lot assignment. In such embodiments, the batch record information can include a selection of inventory associated with a production site and a selection of inventory associated with a pharmaceutical customer. In such embodiments, the one or more programs can further include instructions for, at the first electronic device, receiving the packaging instructions, the bill of materials, and the lot assignment from a user associated with the production facilitator. In such embodiments, the one or more programs can further include instructions for producing a preliminary batch record based on the information input by the user associated with the production facilitator. In one or more embodiments, the one or more programs can further include instructions for, at a second electronic device associated with a pharmaceutical batch customer, displaying a customer user interface that comprises the preliminary batch record and an interface for approving the preliminary batch record. In one or more embodiments, the one or more programs can further include instructions for, at the second electronic device, receiving an approval of the preliminary batch record from a user associated with the pharmaceutical batch customer. In one or more embodiments, the one or more programs can further include instructions for, in accordance with receiving the approval of the preliminary batch record, producing an executable batch record for a producer to execute.

In one or more examples, according to systems of the present disclosure, the user associated with the pharmaceutical batch customer can be configured to approve the preliminary batch record without editing the preliminary batch record. In one or more examples, according to systems of the present disclosure, receiving the packaging instructions can include receiving packaging instructions in a first language, determining whether the first language is used at the production site, and in accordance with a determination that the first language is not used at the production site, generating a translation of the packaging instructions corresponding to a second language used at the production site.

In one or more examples, according to systems of the present disclosure, receiving the lot assignment can include receiving a quantity of output materials to be manufactured, each output material corresponding to one or more input materials, accessing an inventory of a production site, for each output material, assigning a first supplier lot number based on at least one of a first supplier lot size or a first supplier lot expiration date determined from the inventory, and for each input material, assigning a second supplier lot number based on at least one of a second supplier lot size or a second supplier lot expiration date determined from the inventory. In such examples, the one or more programs can further include instructions for generating a second executable batch record for the clinical trial study, including receiving, at the first electronic device, a second lot assignment associated with the production site. In such examples, receiving the second lot assignment can include receiving a second quantity of output materials to be manufactured, each output material corresponding to one or more input materials, accessing the inventory of the production site, for each output material, assigning a third supplier lot number based on at least one of a third supplier lot size or a third supplier lot expiration date determined from the inventory of the production site, and for each input material, assigning a fourth supplier lot number based on at least one of a fourth supplier lot size or a fourth supplier lot expiration date determined from the inventory of the production site. In such examples, the one or more programs can further include instructions for producing a second preliminary batch record that is based on the information input by the user associated with the production facilitator; receiving, from the second electronic device, an approval of the second preliminary batch record; and in accordance with receiving the approval of the second preliminary batch record producing a second executable batch record for the producer to execute.

In one or more examples, the one or more programs can further include instructions for receiving at the first electronic device: customer data associated with the pharmaceutical batch customer, study information for the clinical trial study, the clinical trial study associated with the customer data, and output material information, the output material information corresponding to one or more output materials associated for the clinical trial study. In one or more examples, instructions for receiving the bill of materials can further include importing at least a portion of the output material information to the bill of materials, receiving a quantity associated with each of the one or more output materials and corresponding input materials from the output material information, and assigning a customer identifier based on the customer inventory. In one or more examples, the study information can include packaging sites, study languages, destination countries, output materials, dosage codes, business units, approvers, or a combination thereof. In one or more examples, the output material information can include output materials, input materials, drug categorizations, alternative materials, storage temperature, time out environment details, humidity control details, light control details, label details or a combination thereof.

In one or more examples according to the present disclosure, the executable batch record can be accessed by the user associated with the production facilitator prior to being accessed by the producer. In one or more examples, systems according to the present disclosure can include instructions for causing the first electronic device to present an audit history, the audit history corresponding to a list of actions associated with inputting the batch record information and receiving the approval of the preliminary batch record.

In one or more examples, systems according to the present disclosure can include instructions for, receiving, at the first electronic device, study information for the clinical trial study, the study information comprising one or more customer approvers, the one or more customer approvers comprising the user associated with the pharmaceutical batch customer. The systems can further include instructions for determining whether the batch record information includes blinding information and in accordance with a determination that the batch record information comprises blinding information, causing to present, at the second electronic device, a restricted view of the preliminary batch record that omits the blinding information to the user associated with the pharmaceutical batch customer. In such examples, the user associated with the pharmaceutical batch customer can be configured to be blinded. In such examples, the blinding information can include a medication list, an identification number, a kit number, a start sequence number, an end sequence number, or a combination thereof. In such examples, the one or more customer approvers can further include a second user associated with the pharmaceutical batch customer, wherein the second user associated with the pharmaceutical batch customer is configured to not be blinded. In such examples, systems according to the present disclosure can further include instructions for, in accordance with the determination that the batch record information comprises blinding information, causing to present to the second user associated with the pharmaceutical batch customer, an unrestricted view of the executable batch record that includes the blinding information.

Embodiments of the present disclosure can further include non-transitory computer-readable storage medium storing one or more programs, the one or more programs comprising instructions, which when executed by one or more processors of one or more electronic devices having a display, cause the one or more electronic devices to perform a method. In one or more examples, the method can include at a first electronic device associated with a production facilitator, displaying a user interface (UI) for inputting batch record information, the UI including at least packaging instructions, a bill of materials, and a lot assignment. In such examples, the batch record information can include a selection of inventory associated with a production site and a selection of inventory associated with a pharmaceutical customer. In such examples, the method can further include, at the first electronic device, receiving the packaging instructions, the bill of materials, and the lot assignment from a user associated with the production facilitator. In one or more examples, the method can further include, producing a preliminary batch record based on the information input by the user associated with the production facilitator. In one or more examples, the method can further include, at a second electronic device associated with a pharmaceutical batch customer, displaying a customer user interface that comprises the preliminary batch record and an interface for approving the preliminary batch record. In one or more examples, the method can further include, at the second electronic device, receiving an approval of the preliminary batch record from a user associated with the pharmaceutical batch customer. In one or more examples, the method can further include, in accordance with receiving the approval of the preliminary batch record producing an executable batch record for a producer to execute.

In one or more examples, according to the present disclosure, receiving the lot assignment can include receiving a quantity of output materials to be manufactured, each output material corresponding to one or more input materials, accessing an inventory of a production site, for each output material, assigning a first supplier lot number based on at least one of a first supplier lot size or a first supplier lot expiration date determined from the inventory, and for each input material, assigning a second supplier lot number based on at least one of a second supplier lot size or a second supplier lot expiration date determined from the inventory. In such examples, the one or more programs can further include instructions that cause the one or more electronic devices to generate a second executable batch record for the clinical trial study, including receiving, at the first electronic device, a second lot assignment associated with the production site. In such examples, receiving the second lot assignment can include receiving a second quantity of output materials to be manufactured, each output material corresponding to one or more input materials, accessing the inventory of the production site, for each output material, assigning a third supplier lot number based on at least one of a third supplier lot size or a third supplier lot expiration date determined from the inventory of the production site, and for each input material, assigning a fourth supplier lot number based on at least one of a fourth supplier lot size or a fourth supplier lot expiration date determined from the inventory of the production site. In such examples, the one or more programs can further include instructions that cause the one or more electronic devices to produce a second preliminary batch record that is based on the information input by the user associated with the production facilitator; receive, from the second electronic device, an approval of the second preliminary batch record; and in accordance with receiving the approval of the second preliminary batch record produce a second executable batch record for the producer to execute.

In one or more examples, the one or more programs can further include instructions that cause the one or more electronic devices to receive at the first electronic device: customer data associated with the pharmaceutical batch customer, study information for the clinical trial study, the clinical trial study associated with the customer data, and output material information, the output material information corresponding to one or more output materials associated for the clinical trial study. In one or more examples, instructions for receiving the bill of materials can further include importing at least a portion of the output material information to the bill of materials, receiving a quantity associated with each of the one or more output materials and corresponding input materials from the output material information, and assigning a customer identifier based on the customer inventory. In one or more examples, the study information can include packaging sites, study languages, destination countries, output materials, dosage codes, business units, approvers, or a combination thereof. In one or more examples, the output material information can include output materials, input materials, drug categorizations, alternative materials, storage temperature, time out environment details, humidity control details, light control details, label details or a combination thereof.

In one or more examples according to the present disclosure, the executable batch record can be accessed by the user associated with the production facilitator prior to being accessed by the producer. In one or more examples, the non-transitory computer-readable storage medium according to the present disclosure can include instructions for causing the first electronic device to present an audit history, the audit history corresponding to a list of actions associated with inputting the batch record information and receiving the approval of the preliminary batch record.

In one or more examples according to the present disclosure, the instructions can further cause the one or more electronic devices to receive, at the first electronic device, study information for the clinical trial study, the study information comprising one or more customer approvers, the one or more customer approvers comprising the user associated with the pharmaceutical batch customer. In such examples, the instructions can further cause the one or more electronic devices to determine whether the batch record information includes blinding information and in accordance with a determination that the batch record information comprises blinding information, causing to present, at the second electronic device, a restricted view of the preliminary batch record that omits the blinding information to the user associated with the pharmaceutical batch customer. In such examples, the user associated with the pharmaceutical batch customer can be blinded. In such examples, the blinding information can include a medication list, an identification number, a kit number, a start sequence number, an end sequence number, or a combination thereof. In such examples, the one or more customer approvers can further include a second user associated with the pharmaceutical batch customer, wherein the second user associated with the pharmaceutical batch customer is configured to not be blinded. In such examples, the instructions can further cause the one or more electronic devices to, in accordance with the determination that the batch record information comprises blinding information, cause to present to the second user associated with the pharmaceutical batch customer, an unrestricted view of the executable batch record that includes the blinding information.

Although the disclosure and examples have been fully described with reference to the accompanying figures, it is to be noted that various changes and modifications will become apparent to those skilled in the art. Such changes and modifications are to be understood as being included within the scope of the disclosure and examples as defined by the claims.

The foregoing description, for purpose of explanation, has been described with reference to specific embodiments. However, the illustrative discussions above are not intended to be exhaustive or to limit the invention to the precise forms disclosed. Many modifications and variations are possible in view of the above teachings. The embodiments were chosen and described in order to best explain the principles of the techniques and their practical applications. Others skilled in the art are thereby enabled to best utilize the techniques and various embodiments with various modifications as are suited to the particular use contemplated.

1. A method for generating an executable batch record for a clinical trial study, the method comprising:
at a first electronic device associated with a production facilitator:
displaying a user interface for inputting batch record information comprising packaging instructions, a bill of materials, and a lot assignment, wherein the user interface comprises a selection of inventory associated with a production site and a selection of inventory associated with a pharmaceutical batch customer;
receiving the packaging instructions, the bill of materials, and the lot assignment from a user associated with the production facilitator;
producing a preliminary batch record based on the information input by the user associated with the production facilitator;
at a second electronic device associated with a pharmaceutical batch customer:
displaying a customer user interface that comprises the preliminary batch record and an interface for approving the preliminary batch record;
receiving an approval of the preliminary batch record from a user associated with the pharmaceutical batch customer; and
in accordance with receiving the approval of the preliminary batch record producing an executable batch record for a producer to execute.

2. The method of claim 1, wherein the user associated with the pharmaceutical batch customer is configured to approve the preliminary batch record without editing the preliminary batch record.

3. The method of claim 1, wherein receiving the packaging instructions further comprises:
receiving packaging instructions in a first language;
determining whether the first language is used at the production site;
in accordance with a determination that the first language is not used at the production site, generating a translation of the packaging instructions corresponding to a second language used at the production site.

4. The method of claim 1, wherein receiving the lot assignment further comprises:
receiving a quantity of output materials to be manufactured, each output material corresponding to one or more input materials;
accessing an inventory of a production site;
for each output material, assigning a first supplier lot number based on at least one of a first supplier lot size or a first supplier lot expiration date determined from the inventory; and
for each input material, assigning a second supplier lot number based on at least one of a second supplier lot size or a second supplier lot expiration date determined from the inventory.

5. The method of claim 4, further comprising, generating a second executable batch record for the clinical trial study, comprising:
receiving, at the first electronic device, a second lot assignment associated with the production site comprising:
receiving a second quantity of output materials to be manufactured, each output material corresponding to one or more input materials;
accessing the inventory of the production site;
for each output material, assigning a third supplier lot number based on at least one of a third supplier lot size or a third supplier lot expiration date determined from the inventory of the production site;
for each input material, assigning a fourth supplier lot number based on at least one of a fourth supplier lot size or a fourth supplier lot expiration date determined from the inventory of the production site;
producing a second preliminary batch record that is based on the information input by the user associated with the production facilitator;
receiving, from the second electronic device, an approval of the second preliminary batch record; and
in accordance with receiving the approval of the second preliminary batch record producing a second executable batch record for the producer to execute.

6. The method of claim 1, further comprising, at the first electronic device:
receiving customer data associated with the pharmaceutical batch customer;
receiving study information for the clinical trial study, the clinical trial study associated with the customer data; and
receiving output material information, the output material information corresponding to one or more output materials associated for the clinical trial study.

7. The method of claim 6, wherein receiving the bill of materials further comprises:
importing at least a portion of the output material information to the bill of materials;
receiving a quantity associated with each of the one or more output materials and corresponding input materials from the output material information; and
assign a customer identifier based on the customer inventory.

8. The method of claim 6, wherein the study information comprises packaging sites, study languages, destination countries, output materials, dosage codes, business units, approvers, or a combination thereof.

9. The method of claim 6, wherein the output material information comprises output materials, input materials, drug categorizations, alternative materials, a storage temperature, time out environment details, humidity control details, light control details, label details or a combination thereof.

10. The method of claim 1, wherein the executable batch record is accessed by the user associated with the production facilitator prior to being accessed by the producer.

11. The method of claim 1, further comprising causing to present an audit history, the audit history corresponding to a list of actions associated with inputting the batch record information and receiving the approval of the preliminary batch record.

12. The method of claim 1, further comprising:
receiving, at the first electronic device, study information for the clinical trial study, the study information comprising one or more customer approvers, the one or more customer approvers comprising the user associated with the pharmaceutical batch customer;
determining whether the batch record information comprises blinding information;
in accordance with a determination that the batch record information comprises blinding information, causing to present, at the second electronic device, a restricted view of the preliminary batch record that omits the blinding information to the user associated with the pharmaceutical batch customer,
wherein the user associated with the pharmaceutical batch customer is configured to be blinded.

13. The method of claim 12, wherein the blinding information comprises a medication list, an identification number, a kit number, a start sequence number, an end sequence number, or a combination thereof.

14. The method of claim 12, wherein the one or more customer approvers further comprises a second user associated with the pharmaceutical batch customer, wherein the second user associated with the pharmaceutical batch customer is configured to not be blinded.

15. The method of claim 14, further comprising:
in accordance with the determination that the batch record information comprises blinding information, causing to present to the second user associated with the pharmaceutical batch customer, an unrestricted view of the executable batch record that includes the blinding information.

16. An electronic system, comprising:
one or more processors;
a memory; and
one or more programs, wherein the one or more programs are stored in the memory and configured to be executed by the one or more processors, the one or more programs including instructions for:
at a first electronic device associated with a production facilitator:
displaying a user interface for inputting batch record information comprising at least packaging instructions, a bill of materials, and a lot assignment, wherein the user interface comprises a selection of inventory associated with a production site and a selection of inventory associated with a pharmaceutical batch customer;
receiving the packaging instructions, the bill of materials, and the lot assignment from a user associated with the production facilitator;
producing a preliminary batch record that based on the information input by the user associated with the production facilitator;
at a second electronic device associated with a pharmaceutical batch customer:
displaying a customer user interface that comprises the preliminary batch record and an interface for approving the preliminary batch record;
receiving an approval of the preliminary batch record from a user associated with the pharmaceutical batch customer; and
in accordance with receiving the approval of the preliminary batch record producing an executable batch record for a producer to execute.

17-30. (canceled)

31. A non-transitory computer-readable storage medium storing one or more programs, the one or more programs comprising instructions, which when executed by one or more processors of one or more electronic devices having a display, cause the one or more electronic devices to:
at a first electronic device associated with a batch record facilitator:
display a user interface for inputting batch record information comprising at least packaging instructions, a bill of materials, and a lot assignment, wherein the user interface comprises a selection of inventory associated with a production site and a selection of inventory associated with a pharmaceutical batch customer;
receive the packaging instructions, the bill of materials, and the lot assignment from a user associated with the batch record facilitator;
produce a preliminary batch record that based on the information input by the user associated with the batch record facilitator;
at a second electronic device associated with a pharmaceutical batch customer:
display a customer user interface that comprises the preliminary batch record and an interface for approving the preliminary batch record;
receive an approval of the preliminary batch record from a user associated with the pharmaceutical batch customer; and
in accordance with receiving the approval of the preliminary batch record produce an executable batch record for a producer to execute.

32-45. (canceled)
